(19) United States
(12) Patent Application Publication
(10) Pub. No.: US 2007/0259863 A1
(43) Pub. Date: Nov. 8, 2007

(54) Arylphenylamino-,Arylphenylamide-, and Arylphenylether-Sulfide Derivatives

(75) Inventors: Donovan Chin, Lexington, MA (US); Thomas Durand-Reville, Cambridge, MA (US); Francine S. Farouz, Mercer Island, WA (US); Kerry W. Fowler, Seattle, WA (US); Kevin Guckian, Marlborough, MA (US); Irina Jaconbson, Sammamish, WA (US); Ramesh Kasar, Bellevue, WA (US); Russell C. Petter, Stow, MA (US); Daniel Scott, Weston, MA (US); C. Gregory Sowell, Mukilteo, WA (US); Eugene Thorsett, Kirkland, WA (US); Edward Yin-Shiang Lin, Chestnut Hill, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: ICOS Corporation, Bothell, WA (US); Biogen Idec MA Inc., Cambridge, MA (US)

(21) Appl. No.: 11/587,732

(22) PCT Filed: Apr. 28, 2005
(86) PCT No.: PCT/US05/14778

§ 371 Date: Jul. 3, 2007

Related U.S. Application Data
(60) Provisional application No. 60/565,826, filed on Apr. 28, 2004.
(60) Provisional application No. 60/620,316, filed on Oct. 20, 2004.

(51) Int. Cl. C07D 307/54

(52) U.S. Cl. 514/228.8; 514/255.06; 544/406; 544/63

(57) ABSTRACT

The present invention relates in part to compounds of formulas I and III: 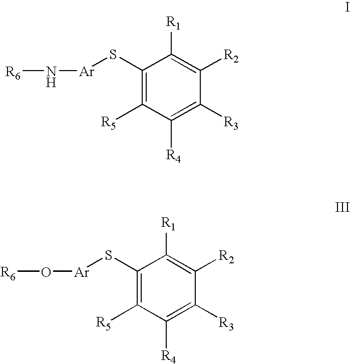 and pharmaceutically-acceptable salts and prodrugs thereof. These compounds can be useful for treating diseases such as inflammatory and immune diseases. The present invention also relates to pharmaceutical compositions comprising these compounds, and to methods of inhibiting inflammation or suppressing immune response in a subject.

### CROSS-REFERENCE TO RELATED APPLICATIONS

This application claims the benefit of U.S. provisional application Ser. No. 60/565,826, filed Apr. 28, 2004, and U.S. provisional application Ser. No. 60/620,316, filed Oct. 20, 2004, the contents of which are incorporated herein by reference in their entirety.

### FIELD OF THE INVENTION

The present invention relates to small molecule LFA-1 antagonists that are useful for treating inflammatory and immune diseases, to pharmaceutical compositions comprising these compounds, to methods of making these compounds, and to methods of inhibiting inflammation, or modulating or suppressing an immune response in a mammal.

### BACKGROUND OF THE INVENTION

Leukocyte function-associated antigen-1 (referred to herein as “LFA-1” and alternatively known as CD11a/CD18) is a heterodimeric cell surface adhesion receptor expressed on all leukocytes. The known counter-receptors for LFA-1 are intracellular adhesion molecules-1, 2, and 3 (ICAM-1, ICAM-2, and ICAM-3). The functional interaction of LFA-1/ICAMs is often associated with a number of inflammatory processes. LFA-1 can serve a dual role in inflammatory responses: it can function as a co-stimulatory molecule during the activation of T cells and can participate in the adhesive interactions associated with the recirculation of leukocytes (for review see; T. A. Springer et al., Nature 1990, 346, 425-434 and M. Lub et al., Immunology Today 1995, 16, 479-483).

Activated T cells are often key mediators in an immune response, functioning either through the secretion of cytokines and chemokines that draw other immune cells to the site of inflammation or through the acquisition of effector functions. The signaling events that lead to T cell activation can arise as a result of the adhesive interaction between T cells and antigen presenting cells (APCs). T cells express specific T cell receptors (TCRs) that recognize their unique cognate antigen as part of an antigen/MHC (major histocompatibility complex) complex on the surface of APCs. The avidity of the TCR interaction is weak and additional adhesive interactions like those conferred by LFA-1/ICAM-1 may be required to stabilize the cell-cell contact and provide co-stimulatory signals. Within the contact site, antigen receptors, adhesion molecules and co-stimulatory molecules are coordinated in a spatio-temporal manner to form a stable “immunological synapse” (IS) that is required for achieving T cell activation. See Monks et al., Nature 395(6697):82-86, 1998; S.-Y. Tseng et al., Curr Opin Cell Biol 14(5):575-580, 2002; M. Krummel et al., Curr Opin Immunol 14(1):66-74, 2002. It is also known that inhibition of LFA-1/ICAM-1 interaction with LFA-1 specific blocking antibodies prevents T cell activation in vitro (Calhoun et al., Transplantation 68:1144, 1999) and in numerous animal models of inflammation.

Inflammation typically results from a cascade of events that includes vasodilation accompanied by increased vascular permeability and exudation of fluid and plasma proteins. This disruption of vascular integrity precedes or coincides with an infiltration of inflammatory cells. Inflammatory mediators generated at the site of the initial lesion serve to recruit inflammatory cells to the site of injury. These mediators (chemokines such as IL-8, MCP-1, MIP-1, and RANTES, complement fragments and lipid mediators) have chemotactic activity for leukocytes and attract the inflammatory cells to the inflamed lesion. These chemotactic mediators, which cause circulating leukocytes to localize at the site of inflammation, require the cells to cross the vascular endothelium at a precise location. This leukocyte recruitment is accomplished by a process called cell adhesion.

Cell adhesion occurs through a coordinately regulated series of steps that allow the leukocytes to first adhere to a specific region of the vascular endothelium and then cross the endothelial barrier to migrate to the inflamed tissue (T. A. Springer, Cell, 76:301-314, 1994; M. B. Lawrence et al., Cell, 65:859-873, 1991; U. von Adrian et al., Proc. Natl. Acad. Sci. USA, 88:7538-7542, 1991; and K. Ley et al., Blood, 77:2553-2555, 1991). These steps are mediated by families of adhesion molecules such as integrins, Ig supergene family members, and selectins, which are expressed on the surface of the circulating leukocytes and on the vascular endothelial cells.

Initially, leukocytes roll along the vascular endothelial cell lining in the region of inflammation. The rolling step may be mediated by either selectin-carbohydrate interactions or integrin-Ig superfamily member interactions between the leukocyte and the luminal surface of inflamed endothelium. The endothelial expression of both selectins and Ig superfamily members are up-regulated in response to the action of inflammatory mediators such as TNF-α and interleukin-1. Rolling decreases the velocity of the circulating leukocyte in the region of inflammation and allows the cells to more firmly adhere to the endothelial cell. The firm adhesion is accomplished by the interaction of integrin molecules that are present on the surface of the rolling leukocytes and their counter-receptors (the Ig superfamily molecules) on the surface of the endothelial cell. The Ig superfamily molecules or cell adhesion molecules (CAMs) are either not expressed or are expressed at low levels on normal vascular endothelial cells. The adhesion process relies on the induced expression of selectins and CAMs on the surface of vascular endothelial cells to mediate the rolling and firm adhesion of leukocytes to the vascular endothelium. The final event in the adhesion process is the extravasation of leukocytes through the endothelial cell barrier and their migration along a chemotactic gradient to the site of inflammation.

The interaction of ICAM-1 (CD54) on endothelial cells with the integrin LFA-1 on leukocytes plays an important role in endothelial-leukocyte contact. Leukocytes bearing high-affinity LFA-1 adhere to endothelial cells through interaction with ICAM-1, initiating the process of extravasation from the vasculature into the surrounding tissues. Thus, an agent that blocks the ICAM-1/LFA-1 interaction suppresses these early steps in the inflammatory response. Consistent with this background, ICAM-1 knockout mice have numerous abnormalities in their inflammatory responses.

Compounds that bind to the inserted-domain (I-domain) of LFA-1, can interrupt endothelial cell-leukocyte adhesion by blocking the interaction of LFA-1 with ICAM-1 and ICAM-3. These compounds can be useful for the treatment or prophylaxis of diseases in which leukocyte trafficking or T-cell activation plays a role, such as acute and chronic inflammatory diseases, autoimmune diseases, tumor metastasis, allograft rejection, and reperfusion injury.

### SUMMARY OF THE INVENTION

The present invention relates to novel compounds and pharmaceutical compositions comprising these compounds. The compounds of the invention can bind to the 1-domain of LFA-1.

In one embodiment, the compounds of this invention are diaromatic sulfides, such as diaryl sulfides or aryl-heteroaryl sulfides, that are substituted with a cinnamide group. The cinnamide functionality may be placed either ortho- or para- to the linking sulfur atom. Appropriate substitution of either or both aromatic rings can be used to modulate a variety of biochemical, physicochemical and pharmacokinetic properties. The cinnamide group can be readily modified; a variety of secondary and tertiary amides can be active, and alternatively a heterocyclic ring may be attached at this position. Modifications of this cinnamide functionality can be useful in modulating physicochemical and pharmacokinetic properties.

In one embodiment, the compounds of the invention are diaryl sulfides and aryl-heteroaryl sulfides that are substituted with a cinnamide group at one aryl, and a secondary amine at the other aryl or heteroaryl. The invention further relates to methods of making diaryl sulfides and aryl-heteroaryl sulfides.

The compounds of the invention can be used to treat diseases such as acute and chronic inflammatory diseases, autoimmune diseases, tumor metastasis, allograft rejection, and reperfusion injury. Thus, certain embodiments of the invention include methods of treating inflammatory and immune diseases, and methods of inhibiting inflammation or suppressing immune response in a mammal.

It is to be understood that both the foregoing general description and the following detailed description are exemplary and explanatory only and are not restrictive of the invention, as claimed.

### DETAILED DESCRIPTION

Definitions

Unless otherwise specified, the chemical groups refer to the unsubstituted and substituted groups.

The term “aldehyde” as used herein refers to the radical —CHO.

The term “aldehyde hydrazone” as used herein refers to the radical —CH═N—NR₁₂R₁₃, where R₁₂ and R₁₃, are independently selected from hydrogen, alkyl, aryl, or cycloalkyl.

The term “alkanoyl” as used herein refers to a carbonyl group attached to an alkyl group.

The term “alkanoylamino” as used herein refers to an alkanoyl group attached to an amino group, e.g., —C(O)-alkyl-amino-.

The term “alkanoylaminoalkyl” as used herein refers to an alkanoylamino group attached to an alkyl group, e.g., —C(O)-alkyl-amino-alkyl-.

The term “alkanoyloxy” as used herein refers to an alkanoyl group attached to an oxygen, e.g., —C(O)-alkyl-O—.

The term “alkanoyloxyalkyl” as used herein refers to an alkanoyloxy group attached to an alkyl group, e.g., —C(O)-alkyl-O-alkyl-.

The term “alkenoxycarbonyl” as used herein refers to an alkenoxy group attached to a carbonyl group, e.g., —O-alkene-C(O)—.

The term “alkenyl” as used herein refers to an unsaturated straight or branched chain of 2-20 carbon atoms having at least one carbon-carbon double bond, such as a straight or branched chain group of 2-12, 2-10, or 2-6 carbon atoms.

The term “alkoxy” as used herein refers to an alkyl group attached to an oxygen. “Alkoxy” groups can optionally contain alkenyl (“alkenoxy”) or alkynyl (“alkynoxy”) groups.

The term “alkoxyalkanoyl” as used herein refers to an alkoxy group attached to an alkanoyl group, e.g., -alkyl-O—C(O)-alkyl-.

The term “alkoxyalkoxy” as used herein refers to an alkoxy group attached to another alkoxy group, e.g., —O-alkyl-O-alkyl-.

The term “alkoxyalkyl” as used herein refers to an alkoxy group attached to an alkyl group, e.g., -alkyl-O-alkyl-.

The term “alkoxyalkylcarbonyl” as used herein refers to an alkoxyalkyl group attached to a carbonyl group, e.g., -alkyl-O-alkyl-C(O)—.

The term “alkoxycarbonyl” as used herein refers to an alkoxy group attached to a carbonyl group, e.g., —C(O)—O-alkyl-.

The term “alkoxycarbonylalkyl” as used herein refers to an alkoxycarbonyl group attached to an alkyl group, e.g., -alkyl-C(O)—O-alkyl-.

The term “alkoxycarbonylamido” as used herein refers to an alkoxycarbonyl group attached to an amido group, e.g., -amido-C(O)—O-alkyl-.

The term “alkyl” as used herein refers to a saturated straight or branched chain group of 1-20 carbon atoms, such as a straight or branched chain group of 1-12, 1-10, or 1-6 carbon atoms.

The term “alkyl(alkoxycarbonylalkyl)amino” as used herein refers to an amino group substituted with one alkyl group and one alkoxycarbonylalkyl group, e.g., -alkyl-C(O)—O-alkyl-amino-alkyl-.

The term “alkylsulfonyl” as used herein refers to an alkyl group attached to a sulfonyl group. “Alkylsulfonyl” groups can optionally contain alkenyl or alkynyl groups.

The term “alkylsulfonylamido” as used herein refers to an alkylsulfonyl group attached to an amido group, e.g., -alkyl-SO₂-amido-.

The term “alkylthio” as used herein refers to an alkyl group attached to a sulfur atom. “Alkylthio” groups can optionally contain alkenyl or alkynyl groups.

The term “alkynyl” as used herein refers to an unsaturated straight or branched chain group of 2-20 carbon atoms having at least one carbon-carbon triple bond, such as a straight or branched chain group of 2-12, 2-10, or 2-6 carbon atoms.

The term “amido” as used herein refers to a radical of the form —R₁₆C(O)N(R₁₄)—, —R₁₆C(O)N(R₁₄)R₁₅—, or —C(O)NR₁₄R₁₅, where R₁₄ and R₁₅ are each independently selected from hydrogen, alkyl, alkanoyl, alkenyl, alkoxy, alkynyl, aryl, carboxy, cycloalkyl, ester, ether, heterocyclyl, hydroxy, ketone, thio, and sulfonyl, and R₁₆ is selected from hydrogen, alkyl, alkoxy, amido, amino, aryl, cycloalkyl, ester, ether, heterocyclyl, halogen, hydroxy, ketone, and thio. The amido can be attached to another group through the carbon, the nitrogen, R₁₄, R₁₅, or R₁₆. The amido also may be cyclic, for example R₁₄ and R₁₅, R₁₆ and R₁₄, or R₁₆ and R₁₅ may be joined to form a 3- to 12-membered ring, such as a 3- to 10-membered ring. The term “amido” encompasses groups such as alkanoylaminoalkyl, amidoalkyl (attached to the parent molecular group through the alkyl), alkylamido (attached to the parent molecular group through the amido), arylamido, amidoaryl, sulfonamide, etc. The term “amido” also encompasses groups such as urea, carbamate, and cyclic versions thereof.

The term “amidoalkoxy” as used herein refers to an amido group attached to an alkoxy group, e.g., -amido-alkyl-O—.

The term “amino” as used herein refers to a radical of the form —NR₁₇R₁₈, —N(R₁₇)R₁₈—, or —R₁₈N(R₁₇)R₁₉— where R₁₇, R₁₈, and R₁₉ are independently selected from hydrogen, alkyl, alkenyl, alkanoyl, alkoxy, alkynyl, amido, amino, aryl, carboxy, cycloalkyl, ester, ether, heterocyclyl, hydroxy, ketone, thio, and sulfonyl. The amino can be attached to the parent molecular group through the nitrogen, R₁₇, R₁₈ or R₁₉. The amino also may be cyclic, for example any two of R₁₇, R₁₈, and R₁₉ may be joined together or with the N to form a 3- to 12-membered ring, e.g., morpholino or piperidinyl. The term “amino” encompasses groups such as aminoalkyl (attached to the parent molecular group through the alkyl), alkylamino (attached to the parent molecular group through the amino), arylamino, aminoaryl, sulfonamino, etc. The term amino also includes the corresponding quaternary ammonium salt of any amino group, e.g., —[N(R₁₇)(R₁₈)(R₁₉)]⁺.

The term “aminoalkanoyl” as used herein refers to an amino group attached to an alkanoyl group, e.g., —C(O)-alkyl-amino-.

The term “aminoalkoxy” as used herein refers to an amino group attached to an alkoxy group, e.g., —O-alkyl-amino-.

The term “aminocarbonyl” as used herein refers to an amino group attached to a carbonyl group.

The term “aminosulfonyl” as used herein refers to an amino group attached to a sulfonyl group.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkyl, aldehyde, alkanoyl, alkoxy, amino, amido, aryl, carboxy, cyano, cycloalkyl, ester, ether, halogen, heterocyclyl, hydroxy, ketone, nitro, sulfonate, sulfonyl, and thio.

The term “arylalkanoyl” as used herein refers to an aryl group attached to an alkanoyl group, e.g., —C(O)-alkyl-aryl- or -alkyl-C(O)-aryl-.

The term “arylalkoxy” as used herein refers to an aryl group attached to an alkoxy group, e.g., —O-alkyl-aryl- or -aryl-O-alkyl-.

The term “arylalkoxycarbonyl” as used herein refers to an arylalkoxy group attached to a carbonyl group.

The term “arylalkyl” as used herein refers to an aryl group attached to an alkyl group.

The term “arylalkylamido” as used herein refers to an arylalkyl group attached to an amido group, e.g., -alkyl-aryl-amido- or -aryl-alkyl-amido-.

The term “arylalkylsulfonyl” as used herein refers to an arylalkyl group attached to an sulfonyl group, e.g., -alkyl-aryl-sulfonyl- or -aryl-alkyl-sulfonyl-.

The term “arylcarboxy” as used herein refers to an aryl group attached to a carboxy group, e.g., -aryl-COOH or salts such as -aryl-COONa.

The term “arylcarboxyamido” as used herein refers to an arylcarboxy group attached to an amido group, e.g., -amido-aryl-COOH or salts such as -amido-aryl-COONa.

The term “aryloxy” as used herein refers to an aryl group attached to an oxygen atom.

The term “aryloxycarbonyl” as used herein refers to an aryloxy group attached to a carbonyl group, e.g., —C(O)—O-aryl- or —O-aryl-C(O)—.

The term “arylsulfonyl” as used herein refers to an aryl group attached to a sulfonyl group, e.g., —S(O)₂-aryl-.

The term “arylsulfonylamido” as used herein refers to an arylsulfonyl group attached to an amido group, e.g., -amido-S(O)₂-aryl-.

The term “carbonyl” as used herein refers to the radical —C(O)—.

The term “carbonyl-containing group” as used herein refers to any group containing the radical —C(O)—. Exemplary carbonyl-containing groups include aldehyde, alkanoyl, arylcarbonyl, amido, ketone, carboxy, cycloalkylcarbonyl, and heterocyclylcarbonyl.

The term “carboxy” as used herein refers to the radical —COOH. The term “carboxy” also includes salts such as —COONa, etc.

The term “carboxyalkoxy” as used herein refers to an alkoxy group attached to a carboxy group, e.g., —O-alkyl-COOH or salts such as —O-alkyl-COONa, etc.

The term “carboxyalkyl” as used herein refers to a carboxy group attached to an alkyl group, e.g., -alkyl-COOH or salts such as -alkyl-COONa, etc. “Carboxylalkyls” can optionally contain alkenyl or alkynyl groups.

The term “carboxyalkylcarbonyl” as used herein refers to a carboxyalkyl group attached to a carbonyl group, e.g., —C(O)-alkyl-COOH or salts such as —C(O)-alkyl-COONa, etc.

The term “carboxyalkylcycloalkyl” as used herein refers to a carboxyalkyl group attached to a cycloalkyl group, e.g., -cycloalkyl-alkyl-COOH or salts such as -cycloalkyl-alkyl-COONa, etc.

The term “carboxyamido” as used herein refers to an amido group attached to a carboxy group, e.g., -amido-COOH or salts such as -amido-COONa, etc.

The term “carboxyamino” as used herein refers to an amino group attached to a carboxy group, e.g., -amino-COOH or salts such as -amino-COONa, etc.

The term “carboxyaminocarbonyl” as used herein refers to a carboxyamino group attached to a carbonyl group, e.g., —C(O)-amino-COOH or salts such as —C(O)-amino-COONa, etc.

The term “carboxycarbonyl” as used herein refers to a carboxy group attached to a carbonyl group, e.g., —C(O)—COOH or salts such as —C(O)—COONa, etc.

The term “carboxycycloalkoxy” as used herein refers to a cycloalkoxy group attached to a carboxy group, e.g., —O-cycloalkyl-COOH or salts such as —C(O)-cycloalkyl-COONa, etc.

The term “carboxycycloalkyl” as used herein refers to a cycloalkyl group attached to a carboxy group, e.g., -cycloalkyl-COOH or salts such as -cycloalkyl-COONa, etc.

The term “carboxycycloalkylalkyl” as used herein refers to a carboxycycloalkyl group attached to an alkyl group, e.g., -alkyl-cycloalkyl-COOH or salts such as -alkyl-cycloalkyl-COONa, etc.

The term “carboxythioalkoxy” as used herein refers to a thioalkoxy group attached to a carboxy group, e.g., —S-alkyl-COOH or salts such as —S-alkyl-COONa, etc.

The term “cyano” as used herein refers to the radical —CN.

The term “cycloalkoxy” as used herein refers to a cycloalkyl group attached to an oxygen, e.g., —O-cycloalkyl-.

The term “cycloalkyl” as used herein refers to a monovalent saturated or unsaturated cyclic, bicyclic, or bridged bicyclic hydrocarbon group of 3-12 carbons derived from a cycloalkane by the removal of a single hydrogen atom, e.g., cyclohexanes, cyclohexenes, cyclopentanes, and cyclopentenes. Cycloalkyl groups may be substituted with alkyl, alkylthio, aldehyde, alkanoyl, alkoxy, amido, amino, aminothiocarbonyl, aryl, arylcarbonyl, arylthio, carboxy, carboxyalkyl, cyano, cycloalkyl, ester, ether, halogen, heterocyclyl, heterocyclylcarbonyl, hydroxy, ketone, nitro, sulfonate, sulfonyl, and thiol. Cycloalkyl groups can be bonded to the parent molecular group through any of its substituents. Cycloalkyl groups can be fused to other cycloalkyl, aryl, or heterocyclyl groups.

The term “cycloalkylalkyl” as used herein refers to a cycloalkyl group attached to an alkyl group, e.g., -alkyl-cycloalkyl-.

The term “ester” refers to a radical having the structure —C(O)O—, —C(O)O—R₂₀—, —R₂₁C(O)O—R₂₀—, or —R₂₁C(O)O—, where 0 is not bound to hydrogen, and R₂₀ and R₂₁ can independently be alkyl, alkenyl, alkynyl, aryl, cycloalkyl, ester, ether, heterocyclyl, ketone, and thio. R₂₁ can be a hydrogen, but R₂₀ cannot be hydrogen. The ester may be cyclic, for example the carbon atom and R₂₀, the oxygen atom and R₂₁, or R₂₀ and R₂₁ may be joined to form a 3- to 12-membered ring. Exemplary esters include alkoxyalkanoyl, alkoxycarbonyl, alkoxycarbonylalkyl, etc. Esters also include carboxylic acid anhydrides and acid halides.

The term “ether” refers to a radical having the structure —R₂₂O—R₂₃—, where R₂₂ and R₂₃ can independently be alkyl, alkenyl, alkynyl, aryl, cycloalkyl, or heterocyclyl. The ether can be attached to the parent molecular group through R₂₂ or R₂₃. Exemplary ethers include alkoxyalkyl and alkoxyaryl groups. Ether also includes polyethers, e.g., where one or both of R₂₂ and R₂₃ are ethers.

The terms “halo” or “halogen” as used herein refer to F, Cl, Br, or I.

The term “haloalkyl” as used herein refers to an alkyl group substituted with one or more halogen atoms. “Haloalkyls” can optionally contain alkenyl or alkynyl groups.

The term “heteroaryl” as used herein refers to a mono-, bi-, or multi-cyclic, aromatic ring system containing one, two, or three heteroatoms such as nitrogen, oxygen, and sulfur. Heteroaryls can be substituted with one or more substituents including alkyl, alkenyl, alkynyl, aldehyde, alkoxy, amido, amino, aryl, carboxy, cyano, cycloalkyl, ester, ether, halogen, heterocyclyl, hydroxy, ketone, nitro, sulfonate, sulfonyl, and thio. Heteroaryls can also be fused to non-aromatic rings.

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkyl, alkenyl, alkynyl, aldehyde, alkylthio, alkanoyl, alkoxy, alkoxycarbonyl, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, oxo, nitro, sulfonate, sulfonyl, and thiol.

Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

Heterocycles also include bridged bicyclic groups where a monocyclic heterocyclic group can be bridged by an alkylene group such as

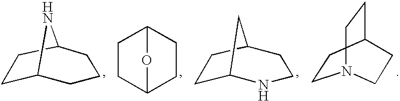

Heterocycles also include compounds of the formula 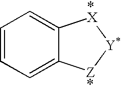
where X* and Z* are independently selected from —CH₂—, —CH₂NH—, —CH₂O—, —NH— and —O—, with the proviso that at least one of X* and Z* is not —CH₂—, and Y* is selected from —C(O)— and —(C(R″)₂)ᵥ—, where R″ is hydrogen or alkyl of one to four carbons, and v is 1-3. These heterocycles include 1,3-benzodioxolyl, 1,4-benzodioxanyl, and 1,3-benzimidazol-2-one.

The term “heterocyclylalkyl” as used herein refers to a heterocyclic group attached to an alkyl group. “Heterocyclylalkyls” can optionally contain alkenyl or alkynyl groups.

The term “heterocyclylalkylcarbonyl” as used herein refers to a heterocyclylalkyl group attached to a carbonyl, e.g., —C(O)-alkyl-heterocyclyl- or -alkyl-heterocyclyl-C(O)—.

The term “heterocyclylalkylsulfonyl” as used herein refers to a heterocyclylalkyl group attached to a sulfonyl, e.g., —SO₂-alkyl-heterocyclyl- or -alkyl-heterocyclyl-SO₂—.

The term “heterocyclylamido” as used herein refers to a heterocyclyl group attached to an amido group.

The term “heterocyclylamino” as used herein refers to a heterocyclyl group attached to an amino group.

The term “heterocyclylcarbonyl” as used herein refers to a heterocyclyl group attached to a carbonyl group.

The term “heterocyclylsulfonyl” as used herein refers to a heterocyclyl group attached to an —SO₂— group.

The term “heterocyclylsulfonylamido” as used herein refers to a heterocyclylsulfonyl group attached to an amido group.

The terms “hydroxyl” and “hydroxyl” as used herein refers to the radical —OH.

The term “hydroxyalkanoyl” as used herein refers to a hydroxy radical attached to an alkanoyl group, e.g., —C(O)-alkyl-OH.

The term “hydroxyalkoxy” as used herein refers to a hydroxy radical attached to an alkoxy group, e.g., —O-alkyl-OH.

The term “hydroxyalkoxyalkyl” as used herein refers to a hydroxyalkoxy group attached to an alkyl group, e.g., -alkyl-O-alkyl-OH.

The term “hydroxyalkyl” as used herein refers to a hydroxy radical attached to an alkyl group.

The term “hydroxyalkylamido” as used herein refers to a hydroxyalkyl group attached to an amido group, e.g., -amido-alkyl-OH.

The term “hydroxyamido” as used herein refers to an amido group attached to a hydroxy radical.

The term “hydroxyamino” as used herein refers to an amino group attached to a hydroxy radical.

The term “ketone” refers to a radical having the structure —R₂₄—C(O)—R₂₅—. The ketone can be attached to another group through R₂₄ or R₂₅. R₂₄ or R₂₅ can be alkyl, alkenyl, alkynyl, cycloalkyl, heterocyclyl or aryl, or R₂₄ or R₂₅ can be joined to form a 3- to 12-membered ring. Exemplary ketones include alkanoylalkyl, alkylalkanoyl, etc.

The term “nitro” as used herein refers to the radical —NO₂.

The term “oxo” as used herein refers to an oxygen atom with a double bond to another atom. For example, a carbonyl is a carbon atom with an oxo group.

The term “perfluoroalkyl” as used herein refers to an alkyl group in which all of the hydrogen atoms have been replaced by fluorine atoms.

The term “phenyl” as used herein refers to a monocyclic carbocyclic ring system having one aromatic ring. The phenyl group can also be fused to a cyclohexane or cyclopentane ring. The phenyl groups of this invention can be substituted with one or more substituents including alkyl, alkenyl, alkynyl, aldehyde, alkoxy, amido, amino, aryl, carboxy, cyano, cycloalkyl, ester, ether, halogen, heterocyclyl, hydroxy, ketone, nitro, sulfonate, sulfonyl, and thio.

The term “sulfonamido” or “sulfonamide” as used herein refers to a radical having the structure —(R₂₇)—N—S(O)₂—R₂₈— or —R₂₆(R₂₇)—N—S(O)₂—R₂₈, where R₂₆, R₂₇, and R₂₈ can be, for example, hydrogen, alkyl, alkenyl, alkynyl, aryl, cycloalkyl, and heterocyclyl. Exemplary sulfonamides include alkylsulfonamides (e.g., where R₂₈ is alkyl), arylsulfonamides (e.g., where R₂₈ is aryl), cycloalkyl sulfonamides (e.g., where R₂₈ is cycloalkyl), heterocyclyl sulfonamides (e.g., where R₂₈ is heterocyclyl), etc.

The term “sulfonate” as used herein refers to the radical —SO₃H. Sulfonate also includes salts such as SO₃Na, etc.

The term “sulfonyl” as used herein refers to a radical having the structure R₂₉SO₂—, where R₂₉ can be alkyl, alkenyl, alkynyl, amino, amido, aryl, cycloalkyl, and heterocyclyl, e.g., alkylsulfonyl.

The term “sulfonylalkylamido” as used herein refers to an alkylamido group attached to a sulfonyl group, e.g. -amido-alkyl-SO₂—.

The term “sulfonylalkylsulfonyl” as used herein refers to an alkylsulfonyl group attached to a sulfonyl group, e.g., —SO₂-alkyl-SO₂—.

The term “thio” as used herein refers to radical having the structure R₃₀S—, where R₃₀ can be hydrogen, alkyl, aryl, cycloalkyl, heterocyclyl, amino, and amido, e.g., alkylthio, arylthio, thiol, etc. “Thio” can also refer to a radical where the oxygen is replaced by a sulfur, e.g., —N—C(S)— is thioamide or aminothiocarbonyl, alkyl-S— is thioalkoxy (synonymous with alkylthio).

“Alkyl,” “alkenyl,” and “alkynyl” groups, collectively referred to as “saturated and unsaturated hydrocarbons,” can be substituted with or interrupted by at least one group selected from aldehyde, alkoxy, amido, amino, aryl, carboxy, cyano, cycloalkyl, ester, ether, halogen, heterocyclyl, hydroxy, ketone, nitro, sulfonate, sulfonyl, thio, O, S, and N.

The term “pharmaceutically-acceptable prodrugs” as used herein represents those prodrugs of the compounds of the present invention that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the invention.

The term “prodrug,” as used herein, represents compounds that are rapidly transformed in vivo to the parent compound of the formulas described herein, for example, by hydrolysis in blood. A discussion is provided in T. Higuchi and V. Stella, Pro-drugs as Novel Delivery Systems, Vol. 14 of the ACS Symposium Series, and in Edward B. Roche, ed., Bioreversible Carriers in Drug Design, American Pharmaceutical Association and Pergamon Press, 1987, both of which are incorporated herein by reference.

Compounds of the present invention can exist as stereoisomers when asymmetric or stereogenic centers are present. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” for clarity in nomenclature, but the skilled artisan will recognize that a structure may denote a chiral center implicitly. Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns.

Geometric isomers can also exist in the compounds of the present invention. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

One embodiment of the present invention provides a compound of formula I:

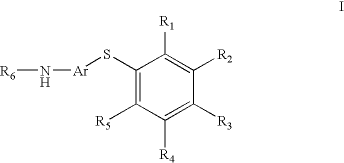

and pharmaceutically-acceptable salts and prodrugs thereof,

wherein R₁, R₂, R₃, R₄, R₅, and R₆ are independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

alternatively, any one or more of R₁, R₂, R₃, R₄, R₅, and R₆ may independently be aminothiocarbonyl,

with the proviso that at least one of R₁ and R₃ is cis-cinnamide or trans-cinnamide defined as

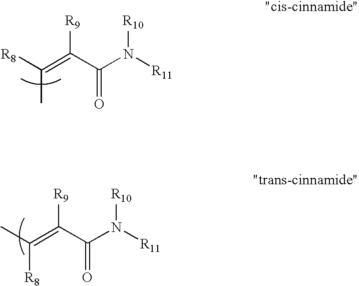

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, sulfonate, sulfonyl, thio, and other carbonyl-containing groups,

wherein
R₁₀ and R₁₁ are each independently selected from hydrogen, alkyl, alkenyl, alkynyl, alkoxy, aryl, arylalkyl, carboxy, cyano, cycloalkyl, ester, ether, heterocyclyl, hydroxy, ketone, nitro, sulfonyl, thio, and other carbonyl-containing groups,
R₁₀ and R₁₁ may independently be alkanoyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein R₁ and R₂, and R₄ and R₅ can be joined to form a 5- to 7-membered cycloalkyl or heterocyclyl ring when R₃ is the cinnamide, and R₂ and R₃, R₃ and R₄, and R₄ and R₅ can be joined to form a 5- to 7-membered ring when R₁ is the cinnamide,

with the proviso that R₆ is not hydrogen, unsubstituted alkyl, unsubstituted saturated cycloalkyl, unsubstituted carboxyalkyl wherein the alkyl is bonded to the NH group of the parent compound, or unsubstituted heterocyclylalkyl wherein the alkyl is bonded to the NH group of the parent compound.

In one embodiment, the carbonyl-containing groups are selected from arylcarbonyl, cycloalkylcarbonyl, and heterocyclylcarbonyl.

In another embodiment, the thio group is selected from alkylthio, arylthio and thiol.

The following alternative embodiments of R₆ can be applied to any of the compounds disclosed herein, e.g., compounds of formula (I) and (III).

In one embodiment, R₆ is selected from alkenyl, alkenoxy, alkynyl, aldehyde, alkanoyl, alkoxy, amido, amino, aryl, aryloxy, a carbonyl-containing group such as a carbonyl bonded to the —NH, carboxy, cyano, ether, ester, halogen, heterocyclyl, hydroxy, ketone, nitro, perfluoroalkyl, substituted alkyl, substituted carboxyalkyl, substituted cycloalkyl, substituted heterocyclylalkyl, sulfonyl, sulfonate, and thio;

In one embodiment, R₆ is selected from aldehyde, alkanoyl, alkenyl, alkenoxy, alkoxy, alkynyl, amido, amino, aminothiocarbonyl, aryl, arylcarbonyl, aryloxy, carboxy, cyano, ester, ether, heterocyclyl, heterocyclylcarbonyl, ketone, nitro, perfluoroalkyl, substituted alkyl, substituted carboxyalkyl, substituted cycloalkyl, substituted heterocyclylalkyl, sulfonyl, and sulfonate.

In one embodiment, R₆ is selected from alkenyl, alkenoxy, alkynyl, aldehyde, alkanoyl, alkoxy, amido, amino, aryl, arylcarbonyl, aryloxy, carboxy, cycloalkylcarbonyl, ether, ester, heterocyclyl, heterocyclylcarbonyl, ketone, nitro, substituted alkyl, substituted cycloalkyl, sulfonyl and sulfonate.

In one embodiment, R₆ is selected from alkanoyl, alkanoylalkyl, amino, amido, aryl, arylalkyl, arylcarbonyl, carboxycycloalkylalkyl, cycloalkylcarbonyl, heterocyclyl, heterocyclylalkyl, heterocyclylcarbonyl, and sulfonyl.

In one embodiment, R₆ is selected from alkanoyl, carbonyl-containing group, amido, aryl, heterocyclyl, sulfonyl, substituted alkyl, substituted cycloalkyl, substituted carboxyalkyl, substituted heterocyclylalkyl (where the heterocyclyl and/or the alkyl is substituted), and thio.

In one embodiment, R₆ can be a substituted alkyl selected from amidoalkyl, aminoalkyl, arylalkyl, carboxycycloalkyl, carboxycycloalkylalkyl, and cycloalkylalkyl. In another embodiment, R₆ can be an amido selected from aminocarbonyl, alkylamido, arylamido, and arylalkylamido. In yet another embodiment, R₆ can be a carbonyl-containing group selected from alkoxycarbonyl, alkoxyalkylcarbonyl, heterocyclylcarbonyl, and heterocyclylalkylcarbonyl. Alternatively, R₆ can be a sulfonyl selected from alkylsulfonyl, aminosulfonyl, arylsulfonyl, arylalkylsulfonyl, heterocyclylsulfonyl, heterocyclylalkylsulfonyl, and sulfonylalkylsulfonyl.

In another embodiment, R₆ is a substituted alkyl, with substitutions selected from carboxycycloalkyl, heterocyclyl, arylcarbonyl, arylhydroxyalkyl and carboxy.

In one embodiment, R₆ is selected from substituted or unsubstituted: alkanoyls, such as acetyl; carboxyalkyls; carboxycycloalkyls, such as carboxycyclohexyl; carboxyalkylcycloalkyls, such as carboxymethyl or carboxyethyl cyclopentyl or cyclohexyl; carboxycycloalkylalkyls, such as carboxycyclohexylalkyl; heterocyclyls, such as tetrahydropyranyls, dioxohexahydro-1λ⁶-thiopyranyls, pyridines, and unsubstituted or N- or C-substituted piperazines and piperidines; heterocyclylcarbonyls; heterocyclylalkylcarbonyls; sulfonyls, such as arylsulfonyls, alkylsulfonyls, and sulfonamides.

In one embodiment, R₆ is an alkanoyl comprising an alkyl group bonded to a carbonyl group, wherein the alkyl group is unsubstituted or substituted with at least one group selected from alkylthio, aldehyde, alkoxy, amido, amino, aminothiocarbonyl, aryl, arylthio, carboxy, cyano, cycloalkyl, ester, ether, halogen, heterocyclyl, hydroxy, ketone, nitro, sulfonate, sulfonyl, and thiol.

In another embodiment, R₆ is an alkanoyl comprising an alkyl group substituted with at least one group selected from alkoxy, alkyl, amino, and heterocyclyl. In another embodiment, R₆ is an alkanoyl that is substituted with at least one group selected from amino and hydroxy.

In one embodiment, R₆ is a cycloalkyl substituted with at least one group selected from alkyl, alkylthio, aldehyde, alkanoyl, alkoxy, amido, amino, aminothiocarbonyl, aryl, arylthio, carboxy, carboxyalkyl, cyano, cycloalkyl, ester, ether, halogen, heterocyclyl, hydroxy, ketone, nitro, sulfonate, sulfonyl, and thiol.

In another embodiment, R₆ is a cycloalkyl substituted with at least one group selected from alkyl, carboxy, and carboxyalkyl.

In one embodiment, R₆ is a heterocyclyl that is unsubstituted or substituted with at least one group selected from alkyl, alkylthio, alkanoyl, alkenyl, alkynyl, aldehyde, alkoxy, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, nitro, oxo, sulfonate, sulfonyl, and thiol.

In another embodiment, R₆ is a heterocyclyl substituted with at least one group selected from alkyl, alkanoyl, amido, arylcarbonyl, cyano, cycloalkyl, cycloalkylcarbonyl, ester, heterocyclylcarbonyl, sulfonyl, and oxo. In another embodiment, R₆ is a heterocyclyl substituted with an alkyl that is substituted with at least one group selected from aryl, alkoxy, alkoxycarbonyl, carboxy, and hydroxy.

In another embodiment, R₆ is a heterocyclyl substituted with at least one group selected from alkanoyl and ester, wherein the carbonyl of the alkanoyl and ester is bonded to a substituent selected from alkenoxy, alkoxyalkoxy, alkoxyalkoxyalkyl, alkoxyalkyl, aminoalkyl, and hydroxyalkyl.

In one embodiment, R₆ is a nonaromatic heterocyclyl bonded to a carbonyl group. In one embodiment the carbonyl group is a —C(O)R_{w} group. In one embodiment, R_{w} is selected from —NHR, —OR, alkyl, -alkyl-OR, and alkyl-OH, and R is selected from alkyl, CN, and —C(O)NH₂. In one embodiment, the heterocyclyl contains a nitrogen in the ring. In another embodiment, the —C(O)R_{w} group defined above is either bonded to the nitrogen of the heterocyclyl or bonded to a carbon in the heterocyclyl ring that is ortho to the nitrogen. Exemplary non-limiting heterocyclyls include pyrrolidine and piperidine.

In one embodiment, R₆ is a nonaromatic heterocyclylcarbonyl group, i.e., —C(O)-heterocyclyl. In one embodiment, the carbonyl is bonded to the nitrogen of the parent compound. In one embodiment, the heterocyclyl contains a nitrogen in the ring. In another embodiment, the nitrogen of the heterocyclyl is bonded to the carbonyl.

In one embodiment, R₆ is selected from an alkylcycloalkyl substituted with a carboxy group, and a cycloalkyl substituted with a carboxy group.

In one embodiment, R₆ is an alkyl substituted with at least one group selected from alkylthio, aldehyde, alkoxy, amido, amino, aminothiocarbonyl, aryl, arylthio, carboxy, cyano, cycloalkyl, ester, ether, halogen, heterocyclyl, hydroxy, ketone, nitro, sulfonate, sulfonyl, and thiol.

In another embodiment, R₆ is an alkyl substituted with at least one group selected from amido, amino, aryl, arylcarbonyl, carboxycycloalkyl, cycloalkyl, and heterocyclyl. In another embodiment, R₆ is an alkyl substituted with a heterocyclyl that is substituted with at least one group selected from alkyl, alkanoyl, and alkoxycarbonyl. In another embodiment, R₆ is an alkyl substituted with an aryl that is substituted with a hydroxy group.

In one embodiment, R₆ is an amido substituted with at least one group selected from hydrogen, alkylthio, alkanoyl, alkenyl, alkoxy, alkyl, alkynyl, amido, amino, aryl, arylthio, carboxy, cycloalkyl, ester, ether, halogen, heterocyclyl, hydroxy, ketone, nitro, sulfonate, sulfonyl, and thiol.

In another embodiment, R₆ is an amido substituted with at least one group selected from alkyl, alkanoyl, aryl, arylalkyl, carboxyalkyl, cycloalkyl, heterocyclylalkyl, and hydroxyalkyl. In another embodiment, R₆ is a thioamido. In another embodiment, R₆ is an amido substituted with an alkanoyl that is substituted with an alkoxy group.

In one embodiment, R₆ is selected from alkanoyl, alkoxycarbonyl, alkoxyalkylcarbonyl, arylalkoxycarbonyl, aryloxycarbonyl, cycloalkylcarbonyl, ester, heterocyclylcarbonyl, heterocyclylalkylcarbonyl, hydroxyalkylcarbonyl, and thiocarbonyl.

In another embodiment, R₆ is selected from aminoalkylcarbonyl, arylcarbonyl, cycloalkylcarbonyl, heterocyclylcarbonyl, heterocyclylalkylcarbonyl, and hydroxyalkylcarbonyl.

In one embodiment, R₆ is a sulfonyl substituted with at least group selected from alkyl, amino, aryl, arylalkyl, haloalkyl, heterocyclyl, heterocyclylalkyl, and sulfonylalkyl.

In one embodiment, any of R₁-R₅ is selected from:

alkyl, which can be selected from alkoxyalkyl, arylalkyl, carboxyalkyl, carboxycycloalkyl, carboxycycloalkylalkyl, cycloalkylalkyl, haloalkyl, and hydroxyalkyl;

alkanoyl, which can be selected from alkanoyloxy, aminoalkanoyl, arylalkanoyl, and hydroxyalkanoyl;

alkenyl, which can be carboxyalkenyl;

alkoxy, which can be selected from alkoxyalkoxy, amidoalkoxy, aminoalkoxy, carboxyalkoxy, carboxycycloalkoxy, and hydroxyalkoxy;

aldehyde, which can be aldehyde hydrazone;

amido, which can be selected from alkylamido, alkylsulfonylamido, alkoxycarbonylamido, aminocarbonyl, arylcarboxyamido, arylsulfonylamido, carboxyamido, carboxyaminocarbonyl, and heterocyclylamido, heterocyclylsulfonylamido, hydroxyamido, sulfonylalkylamido;

amino, which can be selected from carboxyamino, heterocyclylamino, hydroxyamino;

carbonyl-containing group, which can be selected from arylalkoxycarbonyl, aryloxycarbonyl, alkenoxycarbonyl, alkoxycarbonyl, carboxycarbonyl, carboxyalkylcarbonyl, heterocyclylcarbonyl;

ester, which can be selected from alkanoyloxyalkyl;

perfluoroalkyl, which can be selected from trifluoromethyl;

sulfonyl, which can be selected from alkylsulfonyl, aminosulfonyl, arylsulfonyl, arylalkylsulfonyl, heterocyclylsulfonyl, heterocyclylalkylsulfonyl, and sulfonylalkylsulfonyl; and

thio, which can be selected from alkylthio, thioamido, and carboxythioalkoxy.

In one embodiment, R₁ and R₂ are selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, perfluoroalkyl, sulfonyl, sulfonate, thio, and other carbonyl-containing groups.

In another embodiment, R₁ and R₂ are selected from hydrogen, alkyl, halogen, haloalkyl, and nitro.

In one embodiment, R₁ and R₂ are haloalkyl, R₃ is a “trans-cinnamide,” R₄ and R₅ are hydrogen, and Ar is an aryl ring.

In one embodiment, R₈ and R₉ are each independently selected from hydrogen, aldehyde, alkanoyl, alkyl, alkylthio, alkenyl, alkynyl, alkoxy, amido, amino, aryl, arylcarbonyl, arylthio, carboxy, cycloalkyl, ester, ether, heterocyclyl, heterocyclylcarbonyl, ketone, nitro, sulfonate, sulfonyl, and thiol, and

when R₁₀ and R₁₁ are not taken together with N to form a heterocyclyl group bonded to at least one substituent, then R₁₀ and R₁₁ are each independently selected from hydrogen, alkyl, alkylthio, alkanoyl, alkenyl, alkynyl, amido, alkoxy, aryl, arylthio, arylcarbonyl, arylalkyl, carboxy, cyano, cycloalkyl, ester, ether, heterocyclyl, heterocyclylcarbonyl, ketone, nitro, and sulfonyl and thiol.

In one embodiment, R₁₀ and R₁₁ are each independently selected from alkoxyalkyl, alkoxycarbonylalkyl, alkyl, aryl, carboxyalkyl, cycloalkyl, hydroxyalkyl, heterocyclylalkyl, heterocyclyl, and heterocyclylamino.

In one embodiment, R₁₀ and R₁₁ are taken together with N to form a heterocyclyl group bonded to at least one substituent independently selected from alkyl, alkanoyl, alkanoyloxy, alkanoylamino, alkanoyloxyalkyl, alkanoylaminoalkyl, alkoxyalkyl, alkoxycarbonyl, alkoxycarbonylalkyl, amino, alkylsulfonyl, alkylsulfonylaminocarbonyl, arylalkoxycarbonyl, aminoalkyl, aminoalkanoyl, aminocarbonyl, arylsulfonylaminocarbonyl, carboxy, carboxyalkyl, carboxycarbonyl, carboxaldehyde, carboxamido, carboxamidoalkyl, heterocyclyl, heterocyclylalkyl, heterocyclylcarbonyl, heterocyclylalkylaminocarbonyl, hydroxy, hydroxyalkanoyl, hydroxyalkyl, hydroxyalkoxyalkyl, heterocyclylsulfonylaminocarbonyl, and tetrazolyl.

In another embodiment, R₁₀ and R₁₁ are taken together with N to form a heterocyclyl group selected from morpholinyl, piperidinyl, piperazinyl, pyridyl, tetrahydropyridyl, and thiomorpholinyl.

Another embodiment of the present invention provides a compound of formula I:

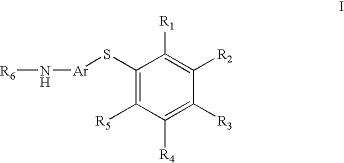

and pharmaceutically-acceptable salts and prodrugs thereof,

wherein R₁, R₂, R₃, R₄, R₅, and R₆ are independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

with the proviso that at least one of R₁ and R₃ is selected from:

(A) substituents of formula IV:

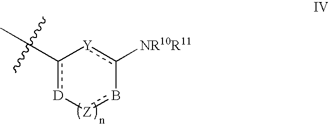

wherein D, B, Y and Z are each independently selected from the group consisting of —CR³¹═, —CR³²R³³—, —C(O)—, —O—, —SO₂—, —S—, —N═, and —NR³⁴—;

n is an integer of zero to three; and

R³¹, R³², R³³ and R³⁴ are each independently selected from the group consisting of hydrogen, alkyl, carboxy, hydroxyalkyl, alkylaminocarbonyl alkyl, dialkylaminocarbonylalkyl and carboxyalkyl; and

(B) cyclopropyl derivatives selected from cis-cyclopropanoic acid, trans-cyclopropanoic acid, cis-cyclopropanamide and trans-cyclopropanamide defined as

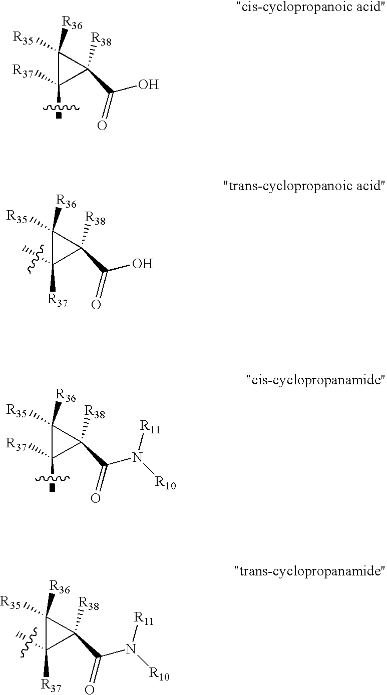

wherein R₃₅ and R₃₆ are each independently selected from the group consisting of hydrogen, alkyl, carboxy, hydroxyalkyl, and carboxyalkyl, and

wherein R₃₇ and R₃₈ are each independently selected from the group consisting of hydrogen, alkyl, carboxyalkyl, alkylaminocarbonylalkyl, and dialkylaminocarbonylalkyl, and

wherein
R₁₀ and R₁₁ⱼ are each independently selected from hydrogen, alkanoyl, alkyl, alkenyl, alkynyl, alkoxy, aryl, arylalkyl, carboxy, cyano, cycloalkyl, ester, ether, heterocyclyl, hydroxy, ketone, nitro, sulfonyl, thio, and other carbonyl-containing groups, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups and

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein R₁ and R₂, and/or R₄ and R₅ can be joined to form a 5- to 7-membered cycloalkyl or heterocyclyl ring when R₃ is selected from substituents of formula IV and cyclopropyl derivatives as defined above, and R₂ and R₃, R₃ and R₄, and/or R₄ and R₅ can be joined to form a 5- to 7-membered ring when R₁ is selected from substituents of formula IV and cyclopropyl derivatives as defined above.

Another embodiment of the present invention provides a compound of formula I:

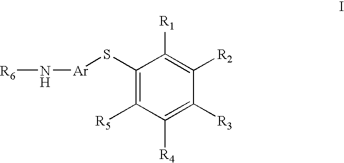

and pharmaceutically-acceptable salts and prodrugs thereof,

wherein R₁, R₂, R₃, R₄, R₅, and R₆ are independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

with the proviso that at least one of R₁ or R₃ is selected from:

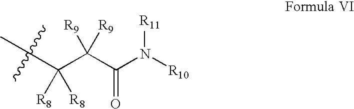

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, sulfonate, sulfonyl, thio, and other carbonyl-containing groups,

wherein
R₁₀ and R₁₁ are each independently selected from hydrogen, alkyl, alkenyl, alkynyl, alkoxy, aryl, arylalkyl, carboxy, cyano, cycloalkyl, ester, ether, heterocyclyl, hydroxy, ketone, nitro, sulfonyl, thio, and other carbonyl-containing groups,
R₁₀ and R₁₁ may independently be alkanoyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein R₁ and R₂, and R₄ and R₅ can be joined to form a 5- to 7-membered cycloalkyl or heterocyclyl ring when R₃ is the substituent of formula IV, and R₂ and R₃, R₃ and R₄, and R₄ and R₅ can be joined to form a 5- to 7-membered ring when R₁ is the substituent of formula IV.

Another embodiment of the present invention provides a compound of formula I:

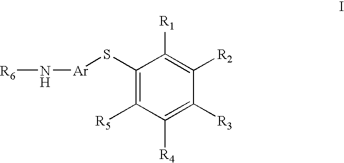

and pharmaceutically-acceptable salts and prodrugs thereof,

wherein R₁, R₂, R₃, R₄, R₅ are each independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups selected from arylcarbonyl, cycloalkylcarbonyl, and heterocyclylcarbonyl,

wherein R₆ is selected from alkyl, aldehyde, alkanoyl, alkenyl, alkenoxy, alkoxy, alkynyl, amido, amino, aminothiocarbonyl, aryl, arylcarbonyl, aryloxy, carboxy, cyano, ester, ether, heterocyclyl, heterocyclylcarbonyl, ketone, nitro, perfluoroalkyl, substituted alkyl, substituted carboxyalkyl, substituted cycloalkyl, substituted heterocyclylalkyl, sulfonyl, and sulfonate,

with the proviso that at least one of R₁ and R₃ is selected from:

cinnamic acids of formula VIII:

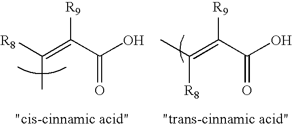

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, heterocyclyl, hydroxy, ketone, nitro, sulfonate, sulfonyl, thio, and other carbonyl-containing groups selected from arylcarbonyl, cycloalkylcarbonyl, and heterocyclylcarbonyl;

wherein:
R₁₀ and R₁₁ are each independently selected from hydrogen, alkyl, alkanoyl, alkenyl, alkynyl, alkoxy, amido, aryl, arylalkyl, carboxy, cyano, cycloalkyl, ester, ether, heterocyclyl, hydroxy, ketone, nitro, sulfonyl, thio, and other carbonyl-containing groups selected from arylcarbonyl, cycloalkylcarbonyl, and heterocyclylcarbonyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups selected from arylcarbonyl, cycloalkylcarbonyl, and heterocyclylcarbonyl, and

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups selected from arylcarbonyl, cycloalkylcarbonyl, and heterocyclylcarbonyl,

wherein R₁ and R₂, and R₄ and R₅ can be joined to form a 5- to 7-membered cycloalkyl, aryl or heterocyclyl ring when R₃ is selected from substituents of formula VII, and R₂ and R₃, R₃ and R₄, and R₄ and R₅ can be joined to form a 5- to 7-membered cycloalkyl, aryl or heterocyclyl ring when R₁ is selected substituents of formula VII.

Another embodiment of the present invention provides a compound of formula I:

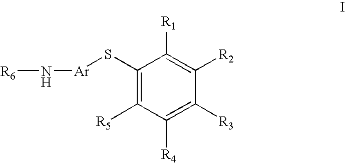

and pharmaceutically-acceptable salts and prodrugs thereof,

wherein R₁, R₂, R₃, R₄, R₅, and R₆ are independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

alternatively, any one or more of R₁, R₂, R₃, R₄, R₅, and R₆ may independently be aminothiocarbonyl,

with the proviso that at least one of R₁ and R₃ is cis-cinnamide or trans-cinnamide defined as

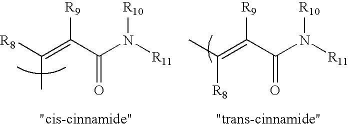

or alternatively, with the proviso that at least one of R₁ and R₃ is selected from A. substituents of formula IV, and B. cyclopropyl derivatives selected from cis-cyclopropanoic acid, trans-cyclopropanoic acid, cis-cyclopropanamide and trans-cyclopropanamide, as defined above,

or alternatively, with the proviso that at least one of R₁ and R₃ is selected from substituents of formula VI, as defined above,

or alternatively, with the proviso that at least one of R₁ and R₃ is selected from substituents of formula VII, as defined above,

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, and other carbonyl-containing groups,

wherein
R₁₀ and R₁₁ are each independently selected from hydrogen, alkyl, alkenyl, alkynyl, alkoxy, aryl, arylalkyl, carboxy, cyano, cycloalkyl, ester, ether, heterocyclyl, hydroxy, ketone, nitro, and other carbonyl-containing groups,
R₁₀ and R₁₁ may independently be alkanoyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups, and

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein R₁ and R₂, and/or R₄ and R₅ can be joined to form a 5- to 7-membered cycloalkyl or heterocyclyl ring when R₃ is selected from cinnamides, substituents of formula IV and cyclopropyl derivatives as defined above, and R₂ and R₃, R₃ and R₄, and/or R₄ and R₅ can be joined to form a 5- to 7-membered ring when R₁ is selected from cinnamides, substituents of formula IV and cyclopropyl derivatives as defined above,

or alternatively, wherein R₁ and R₂, and/or R₄ and R₅ can be joined to form a 5- to 7-membered cycloalkyl or heterocyclyl ring when R₃ is selected from substituents of formula VI as defined above, and R₂ and R₃, R₃ and R₄, and/or R₄ and R₅ can be joined to form a 5- to 7-membered ring when R₁ is selected from substituents of formula VI as defined above,

or alternatively, wherein R₁ and R₂, and/or R₄ and R₅ can be joined to form a 5- to 7-membered cycloalkyl or heterocyclyl ring when R₃ is selected from substituents of formula VII as defined above, and R₂ and R₃, R₃ and R₄, and/or R₄ and R₅ can be joined to form a 5- to 7-membered ring when R₁ is selected from substituents of formula VII as defined above,

with the proviso that:
(i) when R₆ is hydrogen, then R₁₀ or R₁₁ is a cycloalkyl; and
(ii) R₆ is not unsubstituted alkyl, unsubstituted saturated cycloalkyl, unsubstituted carboxyalkyl, or unsubstituted heterocyclylalkyl.

Another embodiment of the present invention provides a compound of formula I:

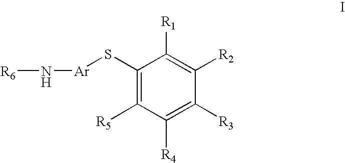

and pharmaceutically-acceptable salts thereof,

wherein R₁, R₂, R₃, R₄, R₅ are each independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,

with the proviso that at least one of R₁ and R₃ is cis-cinnamide or trans-cinnamide is selected from:

cinnamides selected from cis-cinnamide or trans-cinnamide defined as

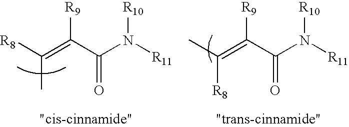

or alternatively, with the proviso that at least one of R₁ and R₃ is selected from A. substituents of formula IV, and B. cyclopropyl derivatives selected from cis-cyclopropanoic acid, trans-cyclopropanoic acid, cis-cyclopropanamide and trans-cyclopropanamide, as defined above, substituents of formula VI, as defined above, and substituents of formula VII, as defined above,

wherein R₆ is selected from alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, heterocyclyl, hydroxy, ketone, nitro, sulfonate, sulfonyl, thio groups selected from alkylthio, arylthio, and thiol, and carbonyl-containing groups selected from arylcarbonyl, cycloalkylcarbonyl, and heterocyclylcarbonyl,

wherein:
R₁₀ and R₁₁ are each independently selected from hydrogen, alkyl, alkanoyl, alkenyl, alkynyl, alkoxy, amido, aryl, arylalkyl, carboxy, cyano, cycloalkyl, ester, ether, heterocyclyl, hydroxy, ketone, nitro, sulfonyl, thio groups selected from alkylthio, arylthio, and thiol, and carbonyl-containing groups selected from arylcarbonyl, cycloalkylcarbonyl, and heterocyclylcarbonyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl, and

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl.

Another embodiment of the present invention provides a compound of formula V:

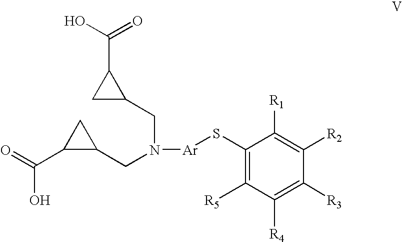

and pharmaceutically-acceptable salts and prodrugs thereof,

wherein R₁, R₂, R₃, R₄, and R₅ are independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,

with the proviso that at least one of R₁ and R₃ is selected from

cinnamides selected from cis-cinnamide and trans-cinnamide defined as

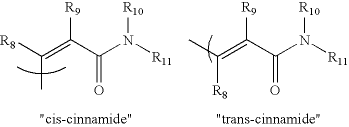

or alternatively, with the proviso that at least one of R₁ and R₃ is selected from A. substituents of formula IV, and B. cyclopropyl derivatives selected from cis-cyclopropanoic acid, trans-cyclopropanoic acid, cis-cyclopropanamide and trans-cyclopropanamide, as defined above, substituents of formula VI, as defined above, and substituents of formula VII, as defined above,

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, heterocyclyl, hydroxy, ketone, nitro, sulfonate, sulfonyl, thio, and other carbonyl-containing groups selected from arylcarbonyl, cycloalkylcarbonyl, and heterocyclylcarbonyl;

wherein:
R₁₀ and R₁₁ are each independently selected from hydrogen, alkyl, alkanoyl, alkenyl, alkynyl, alkoxy, amido, aryl, arylalkyl, carboxy, cyano, cycloalkyl, ester, ether, heterocyclyl, hydroxy, ketone, nitro, sulfonyl, thio groups selected from alkylthio, arylthio, and thiol, and carbonyl-containing groups selected from arylcarbonyl, cycloalkylcarbonyl, and heterocyclylcarbonyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl, and

wherein Ar is selected from aryl and heteroaryl having at least one substituent independently selected from hydrogen, alkyl, alkenyl, alkenoxy, alkynyl,

aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,

wherein R₁ and R₂, and R₄ and R₅ can be joined to form a 5- to 7-membered cycloalkyl, aryl or heterocyclyl ring when R₃ is selected from cinnamides, substituents of formula IV, substituents of formula VI, substituents of formula VII, and cyclopropyl derivatives as defined above, and R₂ and R₃, R₃ and R₄, and R₄ and R₅ can be joined to form a 5- to 7-membered cycloalkyl, aryl or heterocyclyl ring when R₁ is selected from cinnamides, substituents of formula IV, substituents of formula VI, substituents of formula VII, and cyclopropyl derivatives as defined above.

Another embodiment of the present invention provides a compound of formula III:

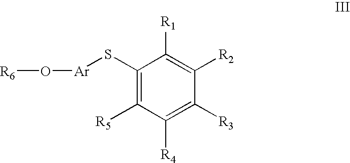

and pharmaceutically-acceptable salts and prodrugs thereof,

wherein R₁, R₂, R₃, R₄, and R₅, are independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, perfluoroalkyl, sulfonyl, sulfonate, thio, and other carbonyl-containing groups;

wherein R₆ is selected from alkenyl, alkenoxy, alkynyl, aldehyde, alkanoyl, alkoxy, amido, amino, aryl, aryloxy, a carbonyl-containing group such as a carbonyl bonded to the —NH, carboxy, cyano, ether, ester, halogen, heterocyclyl, hydroxy, ketone, nitro, perfluoroalkyl, substituted alkyl, substituted carboxyalkyl, cycloalkyl, heterocyclylalkyl, sulfonyl, sulfonate, and thio;

with the proviso that at least one of R₁ and R₃ is cis-cinnamide or trans-cinnamide defined as

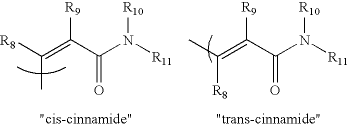

or alternatively, with the proviso that at least one of R₁ and R₃ is selected from A. substituents of formula IV, and B. cyclopropyl derivatives selected from cis-cyclopropanoic acid, trans-cyclopropanoic acid, cis-cyclopropanamide and trans-cyclopropanamide, as defined above,

or alternatively, with the proviso that at least one of R₁ and R₃ is selected from substituents of formula VI, as defined above,

or alternatively, with the proviso that at least one of R₁ and R₃ is selected from substituents of formula VII, as defined above,

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, and other carbonyl-containing groups,

wherein R₁₀ and R₁₁ are each independently selected from hydrogen, alkyl, alkenyl, alkynyl, alkoxy, aryl, arylalkyl, carboxy, cyano, cycloalkyl, ester, ether, heterocyclyl, hydroxy, ketone, nitro, and other carbonyl-containing groups,

R₁₀ and R₁ⱼ may independently be alkanoyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups, and

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups, and

wherein R₁ and R₂, and/or R₄ and R₅ can be joined to form a 5- to 7-membered cycloalkyl or heterocyclyl ring when R₃ is selected from cinnamides, substituents of formula IV and cyclopropyl derivatives as defined above, and R₂ and R₃, R₃ and R₄, and/or R₄ and R₅ can be joined to form a 5- to 7-membered ring when R₁ is selected from cinnamides, substituents of formula IV and cyclopropyl derivatives as defined above,

or alternatively, wherein R₁ and R₂, and/or R₄ and R₅ can be joined to form a 5- to 7-membered cycloalkyl or heterocyclyl ring when R₃ is selected from substituents of formula VI as defined above, and R₂ and R₃, R₃ and R₄, and/or R₄ and R₅ can be joined to form a 5- to 7-membered ring when R₁ is selected from substituents of formula VI as defined above,

or alternatively, wherein R₁ and R₂, and/or R₄ and R₅ can be joined to form a 5- to 7-membered cycloalkyl or heterocyclyl ring when R₃ is selected from substituents of formula VII as defined above, and R₂ and R₃, R₃ and R₄, and/or R₄ and R₅ can be joined to form a 5- to 7-membered ring when R₁ is selected from substituents of formula VII as defined above.

In one embodiment, R₆ is selected from alkanoylalkyl, amino, amido, aryl, arylalkyl, carbonyl-containing group, carboxycycloalkylalkyl, heterocyclyl, heterocyclylalkyl, sulfonyl.

Preparation of Compounds

Preparation of the Compounds of the Invention can be Exemplified by the following schemes and reactions.

In one embodiment, the synthesis of the compound of formula II can be envisioned as piecing together various components A-G, as illustrated below:

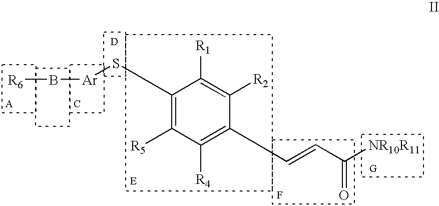

One of ordinary skill in the art will appreciate that the components A-G may be capable of assembly in any order. Component B can be, for example, NH or O. Components F and G can be prepared, for example, by activating a protected acrylic acid a with an —NR₁₀R₁₁-containing reagent to form acrylamide b, as shown in Scheme 1. 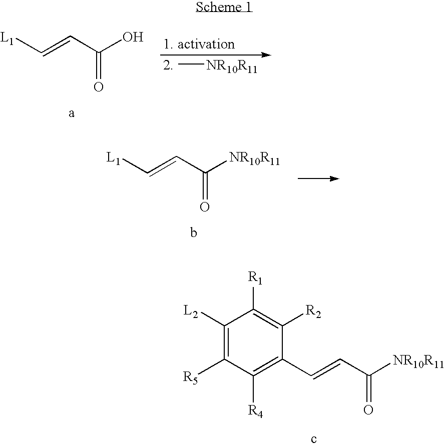
Although Scheme 1 shows the trans form of acrylamide b, one of ordinary skill in the art can appreciate that the cis or trans form can be prepared in any of the described Schemes.

Component E can be prepared by subsequent conversion of the functionalized end of b into cinnamide c. The aryl group can be substituted with any one of substituents R₁, R₂, R₄, R₅, and L₂ prior to or after reacting with b. Exemplary L₁ groups include furyl, hydrogen, triflate, and halogen (e.g., organometallic coupling reactions). Exemplary L₂ groups include hydroxy, sulfonate ester, halogen, and aryl sulfide.

Conversely, an aryl group (or aryl disulfide) can be functionalized with an acrylic acid, as in d, and subsequently reacted to form cinnamide e, as shown in Scheme 2.

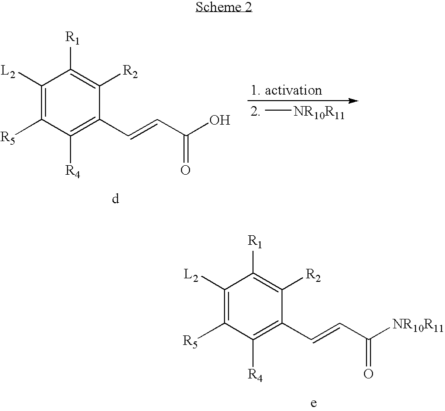

One of ordinary skill in the art will appreciate that component F may be formed simultaneously with component E, for example, by condensation of a benzaldehyde with another carbonyl containing molecule (e.g., aldol or Knoevenagel type condensations).

Components C and D, the aryl or heteroaryl sulfide, can be attached to an aryl group by reacting the aryl group with a thiol or a thiolate. Exemplary aryl sulfide-forming reactions are described in WO 00/59880, pp. 71-90, the disclosure of which is incorporated by reference herein in its entirety. Alternatively, an aryl group, such as a phenol, can be reacted with a sulfonic acid or sulfonate-containing species, to produce a corresponding aryl sulfonic acid ester, as shown in Scheme 3 below. 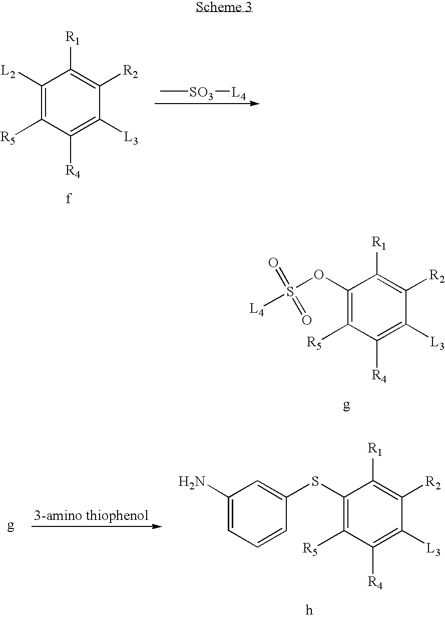
L₂ can be a hydroxy group, or any group capable of reacting with reagents containing the —SO₃-L₄ unit. Exemplary reagents containing the —SO₃-L₄ unit include trifluoromethanesulfonic acid. L₃ can be a cinnamic acid or cis or trans cinnamide or any precursor to a cinnamic acid or cinnamide.

The sulfonic acid ester g in Scheme 3 can be attached to an aryl group by reaction with, for example, a substituted or unsubstituted arylthiol, or any other reagent capable of reacting with g. Scheme 3 illustrates the reaction of sulfonic acid ester g with 3-amino thiophenol to produce the 3-aminophenylsulfanyl unit, h.

One of ordinary skill in the art will appreciate that the secondary amine units, components A and B, i.e., R₆—NH—, can be prepared in a number of ways. In one embodiment, R₆ is selected from:

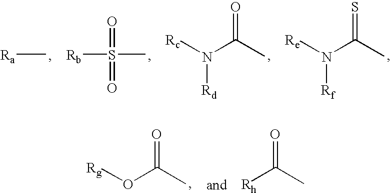

wherein:
Rₐ is selected from alkenyl, alkynyl, aryl, amino, carboxy, cyano, ether, halogen, heterocyclyl, hydroxyl, ketone, nitro, substituted alkyl, substituted cycloalkyl, and thio;
R_{b} is selected from alkyl, alkenyl, alkynyl, alkoxy, amino, amido, aryl, cycloalkyl, carboxyalkyl, cyano, ether, halogen, heterocyclyl, and hydroxy;
R_{c}, R_{d}, Rₑ, and R_{f} are each independently selected from hydrogen, alkyl, alkenyl, alkynyl, aryl, cycloalkyl, and heterocyclyl, or R_{c} and R_{d}, or Rₑ and R_{f} may be joined together to form a 3- to 12-membered ring which can optionally contain one or more atoms selected from N, O, and S and can optionally be substituted;
R_{g} is selected from hydrogen, alkyl, alkenyl, alkynyl, aryl, carboxy, cycloalkyl, ether, heterocyclyl, ketone, and other carbonyl-containing groups; and
Rₕ is selected from hydrogen, alkyl, alkenyl, alkynyl, aryl, amido, carboxy, cycloalkyl, ester, ether, halogen, heterocyclyl, ketone, nitro, sulfonate, sulfonyl, thio, and other carbonyl-containing groups.

In another embodiment, R₆ is selected from:

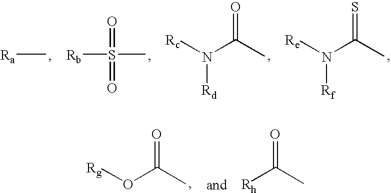

wherein:
Rₐ is selected from alkenyl, alkynyl, aryl, amino, carboxy, cyano, ether, heterocyclyl, ketone, nitro,
   substituted alkyl with at least one substituent selected from alkylthio, aldehyde, alkoxy, amido, amino, aminothiocarbonyl, aryl, arylthio, carboxy, cyano, cycloalkyl, ester, ether, halogen, heterocyclyl, hydroxy, ketone, nitro, sulfonate, sulfonyl, and thiol, and substituted cycloalkyl, with at least one substituent selected from alkyl, alkylthio, aldehyde, alkanoyl, alkoxy, amido, amino, aminothiocarbonyl, aryl, arylthio, carboxy, carboxyalkyl, cyano, cycloalkyl, ester, ether, halogen, heterocyclyl, hydroxy, ketone, nitro, sulfonate, sulfonyl, and thiol;
R_{b′} is selected from alkyl, alkanoyl, alkenyl, alkynyl, alkoxy, amino, amido, aryl, cycloalkyl, carboxyalkyl, cyano, ester, ether, halogen, heterocyclyl, hydroxy, and ketone;
R_{c}, R_{d}, Rₑ, and R_{f} are each independently selected from hydrogen, alkanoyl, alkyl, alkenyl, alkynyl, alkoxy, amino, amido, aryl, carboxy, cycloalkyl, ester, ether, ketone, nitro, and heterocyclyl, or R_{c} and R_{d}, or Rₑ and R_{f} may be joined together to form a substituted or unsubstituted 3- to 12-membered cycloalkyl ring, or a substituted or unsubstituted 3- to 12-membered heterocyclyl ring, which comprises one or more atoms selected from N, O, and S,
   wherein the substituted cycloalkyl or heterocyclyl ring comprises at least one substituent selected from alkyl, alkylthio, alkanoyl, alkenyl, alkynyl, aldehyde, alkoxy, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, nitro, oxo, sulfonate, sulfonyl, and thiol;
R_{g} is selected from hydrogen, alkyl, alkanoyl, aldehyde, alkenyl, alkoxy, alkynyl, amido, amino, aryl, arylcarbonyl, carboxy, cycloalkyl, cycloalkylcarbonyl, ester, ether, heterocyclyl, heterocyclylcarbonyl, and ketone; and
Rₕ is selected from hydrogen, alkyl, alkylthio, alkenyl, alkynyl, alkanoyl, aldehyde, alkoxy, aryl, arylcarbonyl, arylthio, amido, carboxy, cycloalkyl, cycloalkylcarbonyl, ester, ether, halogen, heterocyclyl, heterocyclylcarbonyl, ketone, nitro, sulfonate, sulfonyl, and thiol.

In one embodiment, R₆ can be attached by reacting the NH₂-derivative, h (prepared by, for example, Scheme 3) with an R₆-containing reagent, or an R₆ precursor. For example, R₆ can be attached by reacting h with an R₆-containing halide, carbonyl halide, oxo or ketone, aldehyde, sulfonyl halide (such as an R₆-containing sulfonyl chloride), isocyanate, isothiocyanate, haloformate (such as chloroformate), ester, hydroxy or alcohol, carboxylic acid, and anhydride.

In one embodiment, the NH₂ group on the derivative h can be protected with a protecting group P to form protected amine NHP. The NHP derivative then can be reacted with an R₆ containing reagent or precursor to form an NR₆P derivative followed by deprotection to form the NHR₆ derivative.

In one embodiment, h can be converted to another starting material capable of reacting with an R₆-containing reagent.

In one embodiment, R₆ can be attached to component B prior to formation of the diaryl sulfide. For example, reagent g (prepared by, for example, Scheme 3) can be reacted with an R₆—N(H)-thiophenol. Synthesis of pyrimidine derivatives (Component F of formula II) is shown in Scheme 4. L₂ is as described above. Reaction of methyl ketone i with diethylcarbonate under base catalysis leads to beta-ketoester j. Condensation of j with formamidine gives 4-hydroxypyrimidine k, which can be converted into 4-chloropyrimidine 1. Displacement of the chloride of I by an amine then gives pyrimidine m.

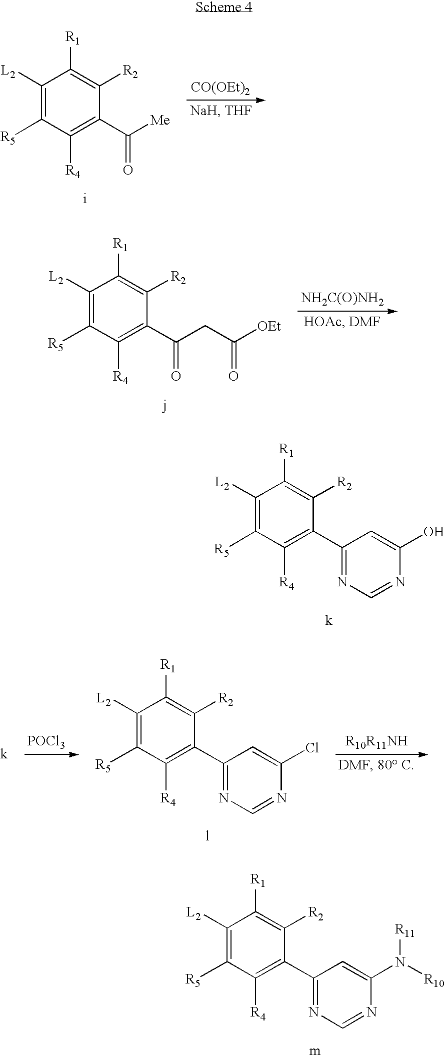

Another route to the 4,6-disubstituted pyrimidines is illustrated in Scheme 5. Transmetallation of n with n-BuLi/ZnCl₂, followed by Pd-catalyzed cross-coupling with 4,6-diiodopyrimidine leads to iodopyrimidine o. Reaction of o with selected amines gives pyrimidine m.

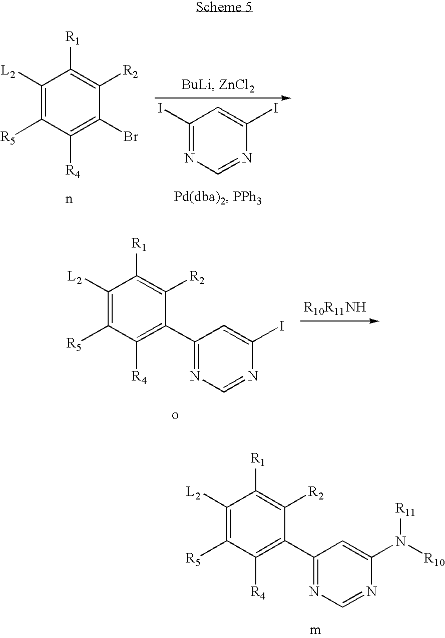

Synthesis of pyridine derivatives (Component F of formula II) can be achieved as shown in Scheme 6. Palladium-catalyzed cross-coupling of properly substituted 1-bromo-4-fluorobenzene p and 4-pyridine boronic acid gives pyridine q. Oxidation of q affords pyridinium oxide r. Fluoride displacement of r with an aryl thiol gives diarylsulfide s. Treatment of s with POCl₃ leads to 2-chloropyridine t. Finally, reaction of t with selected amines gives 2-aminopyridine u.

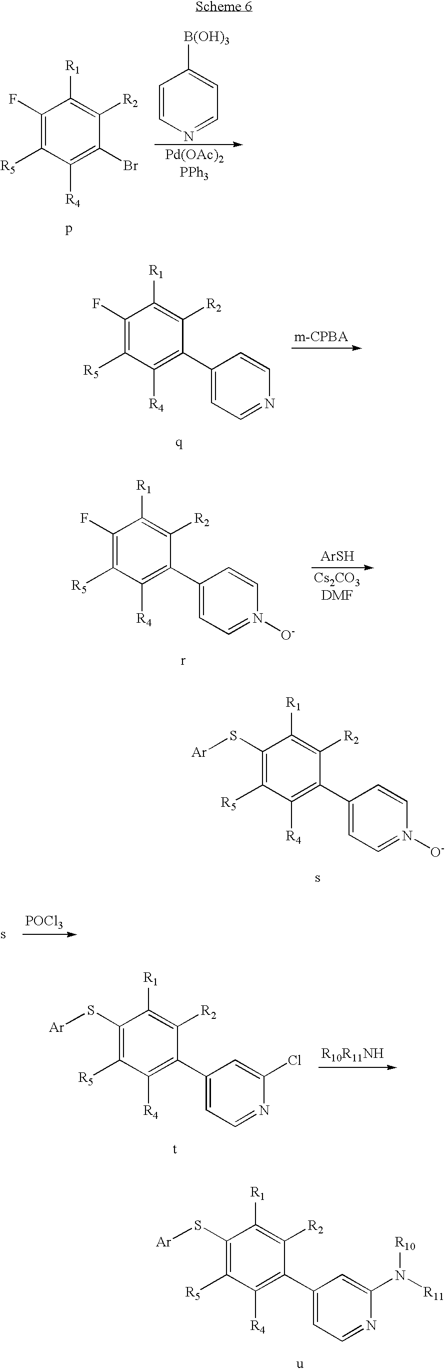

Cyclopropyl derivatives (Component F of formula II) can be accessed by the process shown in Scheme 7, wherein L₂ is as described above. Aldehyde v is treated with an acetate equivalent under basic conditions to afford ester w. Reaction of w with trimethylsulfoxonium iodide in the presence of base (e.g., NaH), followed by hydrolysis of the intermediate ester (using, e.g., NaOH in alcohol), gives cyclopropane acid x. Treatment of x with an amine yields cyclopropanamide y.

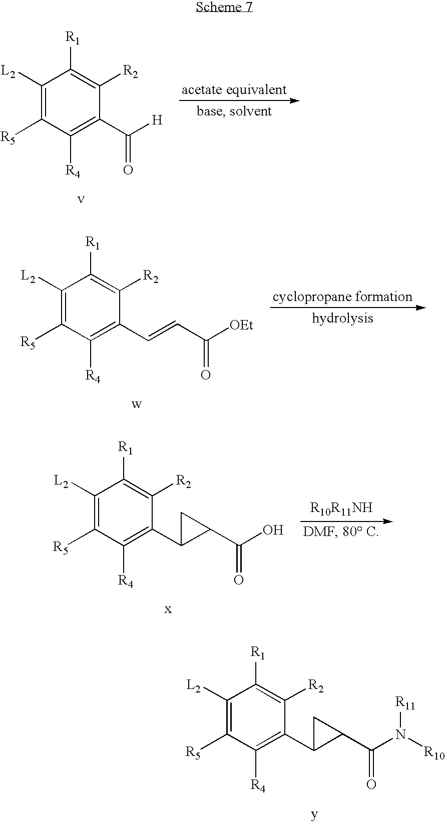

Cyclopropyl derivatives can also be prepared by palladium-mediated coupling of a halo- or trifluorosulfonyl-substituted diarylsulfide with an appropriately substituted alkene. Coupling can be achieved using, e.g., tetrakis(triphenylphosphine)palladium(0), Pd₂(dba)₃, or the like. Cyclopropanation (using, e.g., ethyl diazoacetate and rhodium catalyst) then yields the diarylsulfide cyclopropane derivative. Direct coupling of substituted cyclopropanes with halo- or trifluorosulfonyl-substituted diarylsulfides also affords diarylsulfide cyclopropane derivatives.

Derivatives of Examples 18 and 194 are given below in Table 1.

TABLE 1

| | |
|---|---|
| 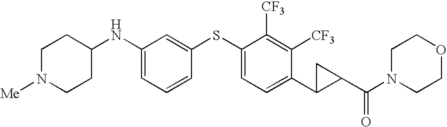 | 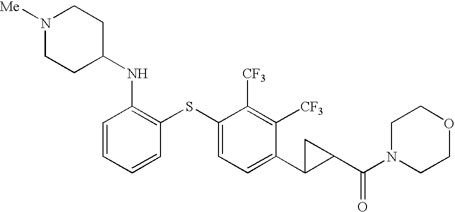 |
| 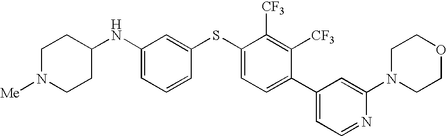 | 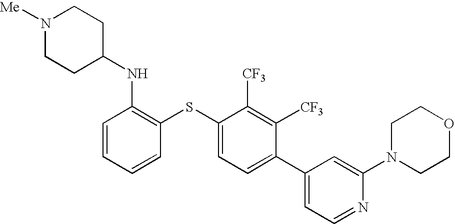 |
| 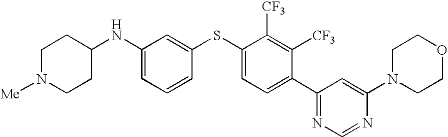 | 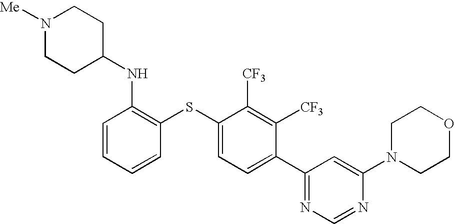 |
| Example 18 derivatives | Example 194 derivatives |

Other substitutions can be performed by the teachings of Publication Nos. WO 00/39081, WO 00/59880, WO 02/02522, and WO 02/02539, the disclosures of which are incorporated by reference herein.

Non-limiting examples of groups of Formula IV include 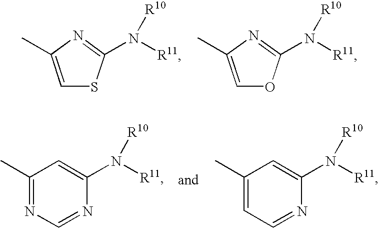
wherein R₁₀ and R₁₁ are as defined above.
Pharmaceutical Compositions

The present invention also provides pharmaceutical compositions comprising compounds of the present invention formulated together with one or more pharmaceutically-acceptable carriers. The pharmaceutical compositions may be specially formulated for topical administration. Alternatively, the pharmaceutical compositions may be specially formulated for oral administration in solid or liquid form, for parenteral injection, for rectal administration, or for vaginal administration. The pharmaceutical compositions may encompass crystalline and amorphous forms of the active ingredient(s).

As used herein, the phrase “pharmaceutically-acceptable carrier” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

The pharmaceutical compositions of this invention can be administered to humans and other animals orally, rectally, parenterally, intracisternally, intravaginally, intraperitoneally, topically (as by powders, ointments, or drops), buccally, or as an oral or nasal spray. The compositions may also be administered through the lungs by inhalation. The term “parenteral administration” as used herein refers to modes of administration, which include intravenous, intramuscular, intraperitoneal, intracisternal, subcutaneous and intraarticular injection and infusion.

Pharmaceutical compositions of this invention for parenteral injection comprise pharmaceutically-acceptable aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, and polyethylene glycol), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservatives, wetting agents, emulsifying agents, and dispersing agents. They may also contain taggants or other anti-counterfeiting agents, which are well known in the art. Prevention of the action of microorganisms may be ensured by the inclusion of various antibacterial and antifungal agents, for example, paraben, chlorobutanol, and phenol sorbic acid. It may also be desirable to include isotonic agents such as sugars, and sodium chloride. Prolonged absorption of the injectable pharmaceutical form may be brought about by the inclusion of agents, which delay absorption such as aluminum monostearate and gelatin.

In some cases, in order to prolong the effect of the drug, it may be desirable to slow the absorption of the drug following subcutaneous or intramuscular injection. This may be accomplished by the use of a liquid suspension of crystalline or amorphous material with poor water solubility. Amorphous material may be used alone or together with stabilizers as necessary. The rate of absorption of the drug then depends upon its rate of dissolution, which in turn, may depend upon crystal size and crystalline form.

Alternatively, delayed absorption of a parenterally administered drug form can be accomplished by dissolving or suspending the drug in an oil vehicle.

Injectable depot forms can be made by forming microencapsulating matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions, which are compatible with body tissues.

The injectable formulations can be sterilized, for example, by filtration through a bacterial-retaining filter, or by incorporating sterilizing agents in the form of sterile solid compositions, which can be dissolved or dispersed in sterile water or other sterile injectable medium just prior to use.

Solid dosage forms for oral administration include capsules, tablets, pills, powders, and granules. Such forms may include forms that dissolve or disintegrate quickly in the oral environment. In such solid dosage forms, the active compound can be mixed with at least one inert, pharmaceutically-acceptable excipient or carrier. Suitable excipients include, for example, (a) fillers or extenders such as starches, lactose, sucrose, glucose, mannitol, and silicic acid; (b) binders such as cellulose and cellulose derivatives (such as hydroxypropylmethylcellulose, hydroxypropylcellulose, and carboxymethylcellulose), alginates, gelatin, polyvinylpyrrolidone, sucrose, and acacia; (c) humectants such as glycerol; (d) disintegrating agents such as sodium starch glycolate, croscarmellose, agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents, such as cetyl alcohol and glycerol monostearate, fatty acid esters of sorbitan, poloxamers, and polyethyleneglycols; (h) absorbents such as kaolin and bentonite clay; (i) lubricants such as talc, calcium stearate, magnesium stearate, solid polyethylene glycols, sodium lauryl sulfate, and mixtures thereof; and (j) glidants such as talc, and silicone dioxide. Other suitable excipients include, for example, sodium citrate or dicalcium phosphate. The dosage forms may also comprise buffering agents.

Solid or semi-solid compositions of a similar type may also be employed as fillers in soft and hard-filled gelatin capsules using such excipients as lactose or milk sugar as well as high molecular weight polyethylene glycols.

Solid dosage forms, including those of tablets, dragees, capsules, pills, and granules, can be prepared with coatings and shells such as functional and aesthetic enteric coatings and other coatings well known in the pharmaceutical formulating art. They may optionally contain opacifying agents and colorants. They may also be in a form capable of controlled or sustained release. Examples of embedding compositions that can be used for such purposes include polymeric substances and waxes.

The active compounds can also be in micro-encapsulated form, if appropriate, with one or more of the above-mentioned excipients.

Liquid dosage forms include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers such as cyclodextrins, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, and perfuming agents. Other ingredients include flavorants for dissolving or disintegrating oral or buccal forms.

Suspensions, in addition to the active compounds, may contain suspending agents such as, for example, ethoxylated isostearyl alcohols, polyoxyethylene sorbitol and sorbitan esters, cellulose or cellulose derivatives (for example microcrystalline cellulose), aluminum metahydroxide, bentonite, agar agar, and tragacanth, and mixtures thereof.

Compositions for rectal or vaginal administration may be suppositories that can be prepared by mixing the compounds of this invention with suitable nonirritating excipients or carriers such as cocoa butter, polyethylene glycol or a suppository wax, that are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes can be formed by lipid monolayer, bilayer, or other lamellar or multilamellar systems that are dispersed in an aqueous medium. Any nontoxic, physiologically-acceptable and metabolizable lipid capable of forming liposomes can be used. The present compositions in liposome form can contain, in addition to a compound of the present invention, stabilizers, preservatives, and excipients. Exemplary lipids include the phospholipids and the phosphatidyl cholines (lecithins), both natural and synthetic.

Methods to form liposomes are known in the art. See, for example, Prescott, Ed., Methods in Cell Biology, Volume XIV, Academic Press, New York (1976), p. 33 et seq.

The compounds of the present invention may be used in the form of pharmaceutically-acceptable salts derived from inorganic or organic acids. By “pharmaceutically-acceptable salt” is meant those salts that are, within the scope of sound medical judgment, suitable for use in contact with the tissues of humans and lower animals without undue toxicity, irritation, and allergic response, and are commensurate with a reasonable benefit/risk ratio. Pharmaceutically-acceptable salts are well known in the art. For example, S. M. Berge, et al. describe pharmaceutically-acceptable salts in J Pharm Sci, 1977, 66:1-19. The salts may be prepared in situ during the final isolation and purification of the compounds of the invention or separately by reacting a free base function with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates, such as dimethyl, diethyl, dibutyl and diamyl sulfates; long-chain halides such as decyl, lauryl, myristyl and stearyl chlorides, bromides and iodides; or arylalkyl halides, such as benzyl and phenethyl bromides and others. Water- or oil-soluble or -dispersible products are thereby obtained.

Examples of acids that may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, and citric acid.

The present invention includes all salts and all crystalline forms of such salts. Basic addition salts can be prepared in situ during the final isolation and purification of compounds of this invention by combining a carboxylic acid-containing group with a suitable base such as the hydroxide, carbonate, or bicarbonate of a pharmaceutically-acceptable metal cation or with ammonia or an organic primary, secondary, or tertiary amine. Pharmaceutically-acceptable basic addition salts include cations based on alkali metals or alkaline earth metals such as lithium, sodium, potassium, calcium, magnesium, and aluminum salts, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

The pharmaceutical composition may also be administered intranasally, topically, or via inhalation. Dosage forms for topical, pulmonary, and nasal administration of a compound of this invention include powders, sprays, ointments, gels, creams, and inhalants. The active compound is mixed under sterile or non-sterile conditions with a pharmaceutically-acceptable carrier and any preservatives, buffers, or propellants that may be required. Ophthalmic formulations, eye ointments, powders and solutions are also contemplated as being within the scope of this invention.

Methods of Treatment

One embodiment of the invention provides a method of treating a subject suffering from diseases chosen from inflammatory diseases, such as acute and chronic inflammatory diseases, and autoimmune diseases.

In one embodiment, the method comprises administering to a subject in need thereof a pharmaceutical composition comprising at least one of the compounds described herein. In one embodiment, the pharmaceutical composition can comprise any one of the compounds described herein as the sole active compound or in combination with another compound, composition, or biological material.

In one embodiment, the invention provides a method of treatment or prophylaxis in which the inhibition of inflammation or suppression of immune response is desired. In another embodiment, the method comprises suppressing an immune response comprising administering to a subject the pharmaceutical composition.

Another embodiment of the invention provides a method of treating a disease mediated at least in part by LFA-1, comprising administering a pharmaceutical composition comprising any compound described herein. In one embodiment, a “disease mediated at least in part by LFA-1” as used herein refers to a disease resulting partially or fully from LFA-1 binding.

Another embodiment of the invention provides a method of treating a disease responsive to an inhibitor of LFA-1, comprising administering a pharmaceutical composition comprising any compound described herein.

In one embodiment, a “subject” as used herein is a mammal, such as a human. In one embodiment, the subject is suspected of having an inflammatory or autoimmune disease, e.g., shows at least one symptom associated with an inflammatory or autoimmune disease. In another embodiment, the subject is one susceptible to having an inflammatory or autoimmune disease, for example, a subject genetically disposed to having the disease.

The terms “treatment,” “therapeutic method,” and their cognates refer to both therapeutic treatment and prophylactic/preventative measures. Those in need of treatment may include individuals already having a particular medical disease as well as those at risk for the disease (i.e., those who are likely to ultimately acquire the disorder). A therapeutic method results in the prevention or amelioration of symptoms or an otherwise desired biological outcome and may be evaluated by improved clinical signs, delayed onset of disease, reduced/elevated levels of lymphocytes and/or antibodies, etc.

The term “immune disease” refers to disorders and conditions in which an immune response is aberrant. The aberrant response can be due to abnormal proliferation, maturation, survival, differentiation, or function of immune cells such as, for example, T or B cells.

Exemplary indications that can be treated by a method according to the invention include, but are not limited to: ischemic-reperfusion injury, such as pulmonary reperfusion injury; stroke; asthma; myocardial infarction; psoriasis, such as chronic plaque, pustular, guttate, and erythrodermic psoriasis; atherosclerosis; atopic dermatitis; hepatitis; adult respiratory distress syndrome; chronic ulceration; lung fibrosis; graft-versus-host disease; chronic obstructive pulmonary disease; Sjögren's syndrome; multiple sclerosis; autoimmune thyroiditis; glomerulonephritis; systemic lupus erythematosus; diabetes; primary biliary cirrhosis; autoimmune uveoretinitis; scleroderma; arthritis, such as psoriatic arthritis and Lyme arthritis; fulminant hepatitis; inflammatory liver injury; thyroid diseases such as Graves' disease; transplant rejection (islets, liver, kidney, heart, etc.); inflammatory lung injury; radiation pneumonitis; inflammatory bowel diseases such as Crohn's disease and ulcerative colitis; inflammatory glomerular injury; radiation-induced enteritis; peripheral artery occlusion; graft rejection; and cancer.

In one embodiment, the present invention provides a method of treatment of any of the indications listed below.

In one embodiment, the present invention provides a method of treating psoriasis. Psoriasis can manifest as one of four forms: chronic plaque, pustular, guttate, and erythrodermic. For example, the role of LFA-1 antagonism can be supported clinically with the use of the monoclonal antibody Efalizumab (Raptiva™) as a treatment for moderate to severe chronic plaque psoriasis (Lebwohl et al., N Engl J Med, 349(21): 2004-2013, 2003. Similarly, small molecule antagonists of LFA-1 may be effective treatments for psoriasis and other inflammatory and autoimmune diseases (Liu, G., Expert Opinion, 11:1383, 2001).

The role of LFA-1 antagonism in treating arthritis can be demonstrated using a murine collagen-induced arthritis model according to the method of Kakimoto et al., Cell Immunol 142:326-337, 1992; a rat collagen-induced arthritis model according to the method of Knoerzer et al., Toxicol Pathol 25:13-19, 1997; a rat adjuvant arthritis model according to the method of Halloran et al., Arthritis Rheum 39:810-819, 1996; a rat streptococcal cell wall-induced arthritis model according to the method of Schimmer et al., J Immunol, 160:1466-1477, 1998; and a SCID-mouse human rheumatoid arthritis model according to the method of Oppenheimer-Marks et al., J Clin Invest 101:1261-1272, 1998.

The role of LFA-1 antagonism in treating fulminant hepatitis can be demonstrated by a murine model of ConA-induced acute hepatic damage (G. Matsumoto et al., J Immunol 169(12):7087-7096, 2002).

The role of LFA-1 antagonism in treating inflammatory liver injury can be demonstrated by a murine liver injury model according to the method of Tanaka et al., J Immunol 151:5088-5095, 1993.

The role of LFA-1 antagonism in treating Sjögren's syndrome can be demonstrated by the studies of Mikulowska-Mennis et al., Am J Pathol 159(2):671-681, 2001. Lymphocyte migration to inflamed lacrimal glands is mediated by vascular cell adhesion molecule-1/alpha(4)beta(1) integrin, peripheral node addressin/l-selectin, and lymphocyte function-associated antigen-1 adhesion pathways.

The role of LFA-1 antagonism in treating autoimmune thyroid diseases such as Graves' disease can be demonstrated by the studies of Arao et al., J Clin Endocrinol Metab, 85(1):382-389, 2000.

The role of LFA-1 antagonism in treating multiple sclerosis can be demonstrated by several animal models demonstrating inhibition of experimental autoimmune encephalomyelitis by antibodies to LFA-1 (E. J. Gordon et al., J Neuroimmunol 62(2):153-160, 1995). Piccio et al. also demonstrated that the firm in vivo arrest of T lymphocytes to inflamed brain venules was LFA-1 dependent (L. Piccio et al., J Immunol, 168(4):1940-1949, 2002).

The role of LFA-1 antagonism in treating autoimmune diabetes can be demonstrated by the method of Fabien et al., Diabetes 45(9):1181-1186, 1996. The role of LFA-1 antagonism in treating autoimmune diabetes can be demonstrated by an NOD mouse model according to the method of Hasagawa et al., Int Immunol 6:831-838, 1994, and by a murine streptozotocin-induced diabetes model according to the method of Herrold et al., Cell Immunol 157:489-500, 1994. Furthermore, several studies have demonstrated improvement in the rate of survival of transplanted islets upon treatment with LFA-1 antagonists (M. Nishihara et al., Transplant Proc 27(1):372, 1995; see also L. Buhler et al., Transplant Proc 26(3):1360-1361, 1994.

The role of LFA-1 antagonism in treating Lyme arthritis can be demonstrated by the method of Gross et al., Science 281:703-706, 1998.

The role of LFA-1 antagonism in treating asthma can be demonstrated by a murine allergic asthma model according to the method of Wegner et al., Science 247:456-459, 1990, or in a murine non-allergic asthma model according to the method of Bloemen et al., Am J Respir Crit. Care Med 153:521-529, 1996.

The role of LFA-1 antagonism in treating inflammatory lung injury can be demonstrated by: a murine oxygen-induced lung injury model according to the method of Wegner et al., Lung 170:267-279, 1992; a murine immune complex-induced lung injury model according to the method of Mulligan et al., Immunol 154:1350-1363, 1995; and a murine acid-induced lung injury model according to the method of Nagase, et al., Am J Respir Crit Care Med 154:504-510, 1996.

The role of LFA-1 antagonism in treating radiation pneumonitis can be demonstrated by a murine pulmonary irradiation model according to the method of Hallahan et al., Proc Natl Acad Sci USA, 94:6432-6437, 1997.

The role of LFA-1 antagonism in treating inflammatory bowel disease can be demonstrated by a rabbit chemical-induced colitis model according to the method of Bennet et al., J Pharmacol Exp Ther, 280:988-1000, 1997.

The role of LFA-1 antagonism in treating inflammatory glomerular injury can be demonstrated by a rat nephrotoxic serum nephritis model according to the method of Kawasaki, et al., J Immunol, 150:1074-1083, 1993.

The role of LFA-1 antagonism in treating radiation-induced enteritis can be demonstrated by a rat abdominal irradiation model according to the method of Panes et al., Gastroenterology 108:1761-1769, 1995.

The role of LFA-1 antagonism in treating reperfusion injury can be demonstrated by the isolated rat heart according to the method of Tamiya et al., Immunopharmacology 29(1):53-63, 1995, or in the anesthetized dog according to the model of Hartman et al., Cardiovasc Res 30(1):47-54, 1995.

The role of LFA-1 antagonism in treating pulmonary reperfusion injury can be demonstrated by a rat lung allograft reperfusion injury model according to the method of DeMeester et al., Transplantation 62(10):1477-1485, 1996, and a rabbit pulmonary edema model according to the method of Horgan et al., Am J Physiol 261(5):H1578-H1584,1991.

The role of LFA-1 antagonism in treating stroke can be demonstrated by: a rabbit cerebral embolism stroke model according the method of Bowes et al., Exp Neurol 119(2):215-219, 1993; a rat middle cerebral artery ischemia-reperfusion model according to the method of Chopp et al., Stroke 25(4):869-875, 1994; and a rabbit reversible spinal cord ischemia model according to the method of Clark et al., Neurosurg 75(4):623-627, 1991.

The role of LFA-1 antagonism in treating peripheral artery occlusion can be demonstrated by a rat skeletal muscle ischemia/reperfusion model according to the method of Gute et al., Mol Cell Biochem 179:169-187, 1998.

The role of LFA-1 antagonism in treating graft rejection can be demonstrated by: a murine cardiac allograft rejection model according to the method of Isobe et al., Science 255:1125-1127, 1992; a murine thyroid gland kidney capsule model according to the method of Talento et al., Transplantation 55:418-422, 1993; a cynomolgus monkey renal allograft model according to the method of Cosimi et al., J Immunol 144:4604-4612, 1990; a rat nerve allograft model according to the method of Nakao et al., Muscle Nerve, 18:93-102, 1995; a murine skin allograft model according to the method of Gorczynski et al., J Immunol 152:2011-2019, 1994; a murine corneal allograft model according to the method of He et al., Opthalmol. V is Sci 35:3218-3225, 1994; and a xenogeneic pancreatic islet cell transplantation model according to the method of Zeng et al., Transplantation 58:681-689, 1994.

The role of LFA-1 antagonism in treating graft-versus-host disease (GVHD) can be demonstrated by a murine lethal GVHD model according to the method of Haming et al., Transplantation 52:842-845, 1991.

The role of LFA-1 antagonism in treating cancers can be demonstrated by a human lymphoma metastasis model (in mice) according to the method of Aoudjit et al., J Immunol 161:2333-2338, 1998.

The role of LFA-1 antagonism in treating atopic dermatitis is supported by the reports of M. Murayama et al., Arch Dermatol Res 289(2):98-103,1997, and S. Kondo et al., Br J Dermatol 131(3):354-9, 1994.

The role of LFA-1 antagonism in treating autoimmune uveoretinitis is supported by the reports of E. Uchio et al., Invest Opthalmol V is Sci 35(5):2626-2631, 1994, and H. Xu et al., J Immunol 172(5):3215-3224, 2004.

The role of LFA-1 antagonism in treating transplant rejection can is supported by the reports of E. K. Nakakura et al., Transplantation 62(5):547-52, 1996, and by R. L. Dedrick et al., Transpl Immunol 9(2-4):181-186, 2002.

Dosing

Actual dosage levels of active ingredients in the pharmaceutical compositions of this invention may be varied so as to obtain an amount of the active compound(s) that is effective to achieve the desired therapeutic response for a particular patient, compositions, and mode of administration. The terms “therapeutically effective dose” and “therapeutically effective amount” refer to that amount of a compound that results in prevention or amelioration of symptoms in a patient or a desired biological outcome, e.g., improved clinical signs, delayed onset of disease, reduced/elevated levels of lymphocytes and/or antibodies, etc. The effective amount can be determined as described herein. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. In one embodiment, the data obtained from the assays can be used in formulating a range of dosage for use in humans.

Generally dosage levels of about 0.1 μg/kg to about 50 mg/kg, such as a level ranging from about 5 to about 20 mg of active compound per kilogram of body weight per day, can be administered topically, orally or intravenously to a mammalian patient. Other dosage levels range from about 1 μg/kg to about 20 mg/kg, from about 1 μg/kg to about 10 mg/kg, from about 1 μg/kg to about 1 mg/kg, from 10 μg/kg to 1 mg/kg, from 10 μg/kg to 100 μg/kg, from 100 μg to 1 mg/kg, and from about 500 μg/kg to about 5 mg/kg per day. If desired, the effective daily dose may be divided into multiple doses for purposes of administration, e.g., two to four separate doses per day. in one embodiment, the pharmaceutical composition can be administered once per day.

The following assays may be used to test compounds of this invention. Unless otherwise indicated, the reagents used in the following examples are commercially available and may be purchased from Sigma-Aldrich Company, Inc. (Milwaukee, Wis., USA) or Alfa Aesar (Ward Hill, Mass., USA).

Assays

ICAM-1/LFA-1 Biochemical Interaction Assay

A biochemical assay may be used to measure the ability of a compound to block the interaction between the integrin LFA-1 and its adhesion partner ICAM-1. Other functionally similar agents and ingredients from alternative sources may be substituted for those described herein.

One hundred microliters (100 μL) of a non-blocking anti-LFA-1 antibody (designated TS2/4.1.1 (ATCC)) at a concentration of 5 μg/mL in 50 mM NaHCO₃/Na₂CO₃ (pH 9.6) plate coating buffer was used to coat wells of Porvair black 96-well microtiter plates overnight at 4° C. The wells were then washed three times with wash buffer (Dulbecco's phosphate-buffered saline (D-PBS) without Ca⁺⁺ or Mg⁺⁺, 0.05% Tween™ 20) and blocked by addition of 200 μL of Superblock® (Pierce Biotechnology, Rockford, Ill.) and further incubated for 1 hour at room temperature. The wells were then washed three times in wash buffer. Recombinant LFA-1 (100 μL of 1.0 μg/mL, Lupher et al., J Immunol 167:1431-1439, 2001) in D-PBS was then added to each well. Incubation was continued for 1 hour at room temperature after which the wells are washed three times with wash buffer. Serial dilutions of compounds being assayed as ICAM-1/LFA-1 antagonists, prepared from 10 mM stock solutions in dimethyl sulfoxide (DMSO), were diluted in D-PBS, 2 mM MgCl₂, 1% Superblock®, 0.05% Tween™ 20, and 50 μL of each dilution was added to duplicate wells. Fifty microliters (50 μL) of 6.0 μg/mL biotinylated recombinant ICAM-1/Ig (R&D Systems, Minneapolis, Minn.) was added to the wells and the plates were incubated at room temperature for 2 hours. The wells were then washed three times with wash buffer and 100 μL of europium-labeled Streptavidin (Wallac Oy) diluted 1:1,500 in Delfia assay buffer (Wallac Oy) are added to the wells. Incubation was allowed to proceed for 1 hour at room temperature. The wells were washed eight times with wash buffer and 100 μL of enhancement solution (Wallac Oy, cat. No. 1244-105) were added to each well. Incubation was allowed to proceed for 5 minutes with constant mixing. Time-resolved fluorimetry measurements were made by using the Victor 1420 Multilabel Counter (Wallac Oy). The percent inhibition of each candidate compound was calculated by using equation (1):

$$\% inhibition = 100 \times [ 1 - ( \frac{average OD w \text{/} compound - background }{average OD w \text{/} o compound - background} ) ] \qquad ( 1 )$$

where “background” refers to wells that were not coated with anti-LFA-1 antibody.

Compounds of the present invention exhibited inhibitory activity in the above assay. In one embodiment, inhibitory activity was indicated by determining the compound concentration at which ICAM-1/LFA-1 interaction is inhibited by 50% (IC₅₀). In certain embodiments, the compounds of the present invention have an IC₅₀ less than or equal to about 1.0 μM, such as an IC₅₀ less than or equal to about 0.1 μM, or an IC₅₀ less than or equal to about 0.01 μM, or less than or equal to about 0.001 μM.

Cell Adhesion Assay

Biologically relevant activity of the compounds in this invention may be confirmed by using a cell-based adhesion assay and mixed lymphocyte reaction assay.

For measurement of inhibitory activity in the cell-based adhesion assay, 96-well microtiter plates were coated with 50 μL of recombinant ICAM-1/Ig (R & D Systems, Inc., Minneapolis, Minn.) at a concentration of 5.0 μg/mL in 50 mM carbonate/bicarbonate buffer, pH 9.6, overnight at 4° C. Alternatively, 96-well microtiter plates can be coated with ICAM-2/Ig (R & D Systems, Inc., Minneapolis, Minn.) or ICAM-3/Ig (R & D Systems, Inc., Minneapolis, Minn.) to determine the potency of compounds in this invention on other known LFA-1 ligands. The wells were then washed twice with 200 μL per well of D-PBS and blocked by the addition of 100 μL of a 1% solution of bovine serum albumin in D-PBS. After a 1-hour incubation at room temperature, the wells were washed once with RPMI-1640 media containing 50% heat-inactivated fetal bovine serum (adhesion media).

To determine the compound concentration at which cell adhesion is inhibited by 50% (IC₅₀), compounds were first serially diluted in DMSO to achieve a range of compound concentrations. Each diluted DMSO stock was then added to ˜0.8 mL of Adhesion Media at a concentration 1.5-fold greater than the final desired compound concentration. The final concentration of DMSO in the ICAM-1/Ig-coated plate did not exceed 0.1%. Two-hundred microliters (200 μL) of the compound diluted in Adhesion Media was added per well to replicate wells (N=3 for each compound concentration) in the microtiter plate. The wells adjacent to the outer edge of the microtiter plate were not used in the cell adhesion assay, but were instead filled with 0.3 mL of Adhesion Media. The plates were then stored at 37° C. in a humidified atmosphere containing 5% CO₂.

A suspension of JY-8 cells (an LFA-1⁺ human EBV-transformed B cell line expressing the IL-8 receptor; Sadhu et al., J Immunol 160:5622-5628, 1998) was prepared containing 0.75×10⁶ cells/mL in Adhesion Media plus 90 ng/mL of the chemokine IL-8 (Peprotech, No. 200-08M). One-hundred microliters (100 μL) of the cell suspension was then added to each well of the microtiter plate containing 200 μL of diluted compound in Adhesion Media. The microtiter plates were incubated for 30 minutes in a humidified 37° C. incubator containing 5% CO₂. The reaction was then halted by the addition of 50 μL of 14% glutaraldehyde/D-PBS, the plates covered with sealing tape (PerkinElmer, Inc., No. 1450-461), and incubated for an additional 90 minutes at room temperature.

To remove non-adherent cells from the microtiter plate, the contents of the wells were gently decanted, and the wells were washed gently with dH₂O. Adherent cells were stained by the addition of 50 μL/well of a 0.5% crystal violet solution. After 5 minutes, the plates were washed by submersion in dH₂O to remove the excess crystal violet solution. Then 70 μL of dH₂O and 200 μL of 95% EtOH were added to each well to extract the crystal violet from the cells. Absorbance was measured 15-60 minutes later at 570 nm in an ELISA plate reader. The percent inhibition of a candidate compound was calculated by using equation (1) above.

All compounds of the present invention showed an IC₅₀ in this assay of no more than 10 μM.

T Cell Proliferation Assay

A mixed lymphocyte reaction (MLR) may be used to determine the effect of small molecule antagonists of LFA-1 on T cell proliferation and activation. One-way MLRs can provide a measure of the mitogenic response of T lymphocytes from one individual to the alloantigens present on the cells of a second individual, provided they are mismatched in histocompatibility loci. This proliferative response can be initiated by the engagement of the T cell receptor and several co-stimulatory receptors present on T lymphocytes. LFA-1 is one of the co-stimulatory receptors. (See M. C. Wacholtz et al., J Exp Med 170(2):431-448, 1989; see also G. A. Van Seventer et al., J Immunol 144(12):4579-4586, 1990). The LFA-1 ligand ICAM-1 can provide a costimulatory signal for T cell receptor-mediated activation of resting T cells. (Blockade of LFA-1 by antibodies to CD11a blocks T cell activation and proliferation in a MLR. K. Inaba et al., J Exp Med 1; 165(5):1403-17, 1987; G. A. Van Seventer et al., J Immunol 149(12):3872-80, 1992). Costimulation of T cell receptor/CD3-mediated activation of resting human CD4+ T cells by LFA-1 ligand ICAM-1 can involve prolonged inositol phospholipid hydrolysis and sustained increase of intracellular Ca²⁺ levels.

Experimental design of MLRs is well established. (See, e.g., Current Protocols in Immunology, Ed. John E. Colligan et al., John Wiley & Sons, 1999). Human peripheral blood mononuclear cells were isolated from ˜60 mL of blood from two different donors by using heparin as an anticoagulant (20 U/mL, final concentration). The blood was diluted three-fold with RPMI-1640 containing 25 mM Hepes (pH 7.4), 2 mM L-glutamine, 2 g/L sodium bicarbonate, 10 U/mL penicillin G, and 10 μg/mL streptomycin. In 50 mL polypropylene centrifuge tubes, aliquots of approximately 25 mL of diluted blood were layered onto 12.5 mL of Histopaque®-1077 (Sigma Corp., No. 1077) and the tubes were centrifuged at 514×g for 30 minutes at room temperature without braking. After centrifugation, the buffy coat containing the peripheral blood mononuclear cells was transferred to a new 50 mL tube and diluted approximately five-fold with RPM-1640 and mixed by gentle inversion. Tubes were then centrifuged at 910×g for 10 minutes at room temperature. The supernatant was aspirated, and the cells were re-suspended in MLR media (RPMI-1640 containing 50% fetal bovine serum (HyClone), 25 mM Hepes (pH 7.4), 2 mM L-glutamine, 2 g/L sodium bicarbonate, 10 U/mL penicillin G, and 10 μg/mL streptomycin) and adjusted to a final concentration of 2×10⁶ cells/mL.

To allow for a one-way proliferative response, the cells from one blood donor (referred to as “the donor”) were irradiated with approximately 1500 rad emitted from a ¹³⁷Cs source (Mark I Irradiator, Shepard and Associates). Irradiated cells remained viable during the course of the MLR but did not proliferate in response to alloantigens. Non-irradiated cells from a second blood donor (referred to as “the responder”) were added 1:1 (50 μL:50 μL) with irradiated cells from the donor to a 96-well round-bottom microtiter plate. Each well also contained 100 μL of either LFA-1 inhibitor or MLR media alone in the case of the positive control. A negative control, designed to represent an autologous antigen response, of 50 μL of irradiated responder cells and 50 μL of non-irradiated responder cells was also present on each MLR plate.

LFA-1 inhibitors, e.g., anti-CD11a antibodies or small-molecule antagonists, were prepared at twice their final desired concentration in MLR media. Small molecule antagonists were typically tested at final concentrations ranging from 10 to 0.002 μM. Anti-CD11a monoclonal antibodies were typically tested at final concentrations ranging from 2,000 to 16 ng/mL. Six replicate wells were used for each concentration of LFA-1 inhibitor. The wells adjacent to the outer edges of the microtiter plate were not used for a MLR, but were instead filled with 200 μL of MLR media. The assay plates were then incubated at 37° C. in a 5% CO₂ atmosphere.

For each inhibitor that was tested, three identical MLR plates were prepared. The supernatants from two plates were harvested on days three and five following initiation of the MLR for cytokine analysis. The supernatant from each of the six replicate wells harvested on either day three or day five was pooled and stored at −70° C. in a 96-deepwell polypropylene plate covered with a silicone gasket. To assess T cell proliferation on the third MLR plate, 1 μCi of ³H-thymidine (New England Nuclear, No. NET-027) in 20 μL of MLR media was added per well of the MLR microtiter plate on day four. Twenty-four hours later, the cells from each well were harvested onto glass fiber filter plates (PerkinElmer Unifilter-96 GF/C plates, No. 6005147) using a Packard FilterMate Harvester (Packard Instrument Co.). ³H-Thymidine incorporation was measured as counts per minute (cpm) in a scintillation counter (Packard TopCount-NXT™).

The mean cpm from 6 replicate wells was determined for each inhibitor concentration, as well as positive (allogeneic MLR) and negative (autologous MLR) controls. The mean cpm obtained from the autologous MLRs was designated as background counts, and was subtracted from the mean cpm obtained from the positive control and LFA-1 inhibitor samples. The percent proliferation is normalized to the mean cpm obtained in the absence of inhibitor, i.e., the allogeneic MLR by using equation (2):

$$\% proliferation = 100 \times \frac{( mean inhibitor cpm - mean background cpm )}{( mean positive control cpm - mean background cpm )} \qquad ( 2 )$$

In one embodiment, the potency of the compound is indicated by determining the compound concentration at which cell proliferation is inhibited by 80% (EC₈₀). In one embodiment, wherein upon subjecting the compound to a T cell proliferation assay, the compound exhibits an EC₈₀ of less than or equal to about 3.0 μM, such as an EC₈₀ of less than or equal to about 0.3 μM or an EC₈₀ Of less than or equal to about 0.03 μM.

Cytokine measurements, e.g., IL-2, IFN-γ, and TNF-α, were also determined on MLR supernatants harvested on day 3 (IL-2) and day 5 (IFN-α and TNF-α). Cytokine concentrations were determined by using ELISA kits (Biosource International) based on standard curves generated with purified cytokine standards diluted in MLR media. The background level of cytokine production was established as the mean cytokine concentration of the autologous MLR. The mean cytokine concentration of the allogeneic MLR in the absence of inhibitor was used as the positive control. The level of cytokine present in the inhibitor-treated MLRs relative to the positive control represented the percent maximal response and was calculated by using equation (3):

$$\% Maximal response = 100 \times \frac{\begin{matrix}( mean inhibitor cytokine conc . - \\ mean background cytokine conc . )\end{matrix}}{\begin{matrix}( mean positive control cytokine conc . - \\ mean background cytokine conc . )\end{matrix}} \qquad ( 3 )$$

### EXAMPLE 1

### 3-Furan-2-yl-1-morpholin-4-yl-propenone

Furylacrylic acid (25 g, 181 mmol) was added to 200 mL of methylene chloride and the reaction was cooled to 0° C. Thionyl chloride (19.8 mL, 272 mmol) was then added over 15 minutes. The solution was allowed to warm to room temperature overnight, and the reaction went from cloudy to clear the next morning. In a separate flask 150 mL of methylene chloride and morpholine (47.5 mL, 545 mmol) were added and the flask was brought to 0° C. The solution containing the furan was then added dropwise by addition funnel to the cooled solution containing the morpholine. After addition the solution was allowed to warm to room temperature and stir for 1.5 h. The reaction was then extracted twice with 1 N HCl, twice with brine, and dried over sodium sulfate. The organic layer was then decolorized by carbon and concentrated to dryness. This yielded a pale yellow solid (87%, 32.5 g, 156 mmol). ¹H NMR (CDCl₃, 300 MHz) δ 3.60-3.78 (m, 8H), 6.48 (q, J=2 Hz, 1H), 6.58 (d, J=3 Hz, 1H), 6.78 (d, J=16 Hz, 1H), 7.45-7.53 (m, 2H); MS (ESI (+)) m/z 208.1 (M+H+).

### EXAMPLE 2

### 3-(4-Hydroxy-2,3-bis-trifluoromethyl-phenyl)-1-morpholin-4-yl-propenone

A solution of 3-furan-2-yl-1-morpholin-4-yl-propenone (32 g, 106 mmol) in 80 mL of dichloroethane was prepared and placed in a Parr stirred reactor. The reactor was cooled to −78° C. and 1,1,1,4,4,4-hexafluoro-2-butyne (50 g, 219 mmol) gas was added. The was allowed to come to room temperature over two hours then the reaction was heated to 115° C. for 23 hr. HPLC analysis showed the disappearance of the starting material. The dichloroethane solution was then concentrated and brought up in 180 mL of dichloroethane. Boron trifluoride diethyl etherate (29.65 mL, 234 mmol) was added to the reaction and refluxed for three hours. The crude was concentrated and purified by column chromatography using 2:3 ethyl acetate/hexanes (47%, 27 g, 73 mmol). ¹H NMR (CDCl₃, 300 MHz) δ 3.60-3.78 (m, 8H), 6.47 (d, J=15 Hz, 1H), 7.08 (d, J=8 Hz, 1H), 7.44 (d, J=8 Hz, 1H), 7.73-7.84 (m, 1H).

### EXAMPLE 3

### Trifluoromethanesulfonic acid 4-(3-morpholin-4-yl-3-oxo-propenyl)-2,3-bis-trifluoromethyl-phenyl ester

3-(4-Hydroxy-2,3-bis-trifluoromethyl-phenyl)-1-morpholin-4-yl-propenone (8.8 g, 23.8 mmol) was dissolved in 100 mL of dichloromethane and 6 mL of pyridine was added. The reaction was cooled to 0° C. and triflic anhydride was added slowly. After warming to room temperature the reaction washed twice with cold 1 N HCl, twice with a cold saturated bicarbonate solution, and then dried with sodium sulfate, filtered and concentrated. (80%, 9.2 g). ¹H NMR (CDCl₃, 300 MHz) δ 3.57-3.78 (m, 8H), 6.66 (d, J=15 Hz, 1H), 7.65 (d, J=8 Hz, 1H), 7.78 (d, J=8 Hz, 1H), 7.85-7.93 (m, 1H).

### EXAMPLE 4

### 3-[4-(3-Amino-phenylsulfanyl)-2,3-bis-trifluoromethyl-phenyl]-1-morpholin-4-yl-propenone

3-Amino thiophenol (2.75 mL, 25.7 mmol) was dissolved in 86 mL of tetrahydrofuran (THF) and placed at −17° C. Lithium t-butoxide (2.0 g, 25.7 mmol) was added and the reaction was allowed to warm to room temperature before being placed back at 0° C. In a separate round bottom flask, trifluoromethanesulfonic acid 4-(3-morpholin-4-yl-3-oxo-propenyl)-2,3-bis-trifluoromethyl-phenyl ester was dissolved in 53 mL of THF and placed at −78° C. The deprotonated 3-amino-thiophenol was then cannulated into the round bottom flask containing trifluoromethanesulfonic acid 4-(3-morpholin-4-yl-3-oxo-propenyl)-2,3-bis-trifluoromethyl-phenyl ester at −78° C. After one hour at −78° C. the starting material was consumed. The reaction was concentrated and purified by column chromatography using 2% MeOH/98% dichloromethane (DCM) (61%, 5.21 g). ¹H NMR (DMSO-d₆, 300 MHz) δ 3.57-3.75 (m, 8H), 5.45 (s, 2H), 6.70-6.74 (m, 3H), 7.18 (t, J=8 Hz, 1H), 7.23 (d, J=15 Hz, 1H), 7.36 (d, J=9 Hz, 1H), 7.65-7.75 (m, 1H), 8.05 (d, J=9 Hz, 1H); MS (ESI (+)) m/z 477.3 (M+H+).

### EXAMPLE 5

### 3-[4-(3-Methylamino-phenylsulfanyl)-2,3-bis-trifluoromethyl-phenyl]-1-morpholin-4-yl-propenone

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 240 μL of dimethylformamide (DMF) then methyl iodide (10.61 μL, 0.26 mmol) and potassium carbonate (14 mg, 0.10 mmol) were added. The reaction proceeded very slowly at room temperature to about 50% conversion over three days. 40% was monomethylated and 10% was dimethylated. The crude reaction was diluted with DMF and purified by preparative HPLC to give the pure mono-methylated product. MS (ESI (+)) m/z 491.1 (M+H⁺).

### EXAMPLE 6

### Cis 4-{3-[4-(3-morpholin-4-yl-3-oxo-propenyl)-2,3-bis-trifluoromethyl-phenylsulfanyl]-phenylamino}-cyclohexanecarboxylic acid

The product of Example 4, 3-[4-(3-amino-phenylsulfanyl)-2,3-bis-trifluoromethyl-phenyl]-1-morpholin-4-yl-propenone (1.5 g, 3.15 mmol), was dissolved in 27 mL of dichloroethane and 1.1 mL of acetic acid was added. Ethyl 4-oxocyclohexanecarboxylate (1.6 mL, 9.45 mmol) then sodium triacetoxyborohydride (2.67 g, 12.6 mmol) were added and the reaction was allowed to stir overnight. HPLC analysis showed the appearance of the two product peaks in a 3:7 ratio. The reaction product was extracted twice with sodium bicarbonate and twice with brine before drying with magnesium sulfate and concentration to give a yellow oil. The oil was dissolved in DMSO and preparative HPLC was utilized to separate the two isomers. Each isomer was then hydrolyzed in 2:1 THF/H₂O by adding 2 N LiOH until basic. The individual solutions were then concentrated and brought up in water. 1 N HCL was then added until the pH reached approximately 4 and this resulted in the precipitation of the product. The product was then filtered and washed several times with water. The isomeric products were identified as cis and trans about the cyclohexane ring by solving X-ray cocrystal structures with LFA-1. The cis compound elutes last on the HPLC and is the major product. Cis: ¹H NMR (CDCl₃, 300 MHz) δ 1.56-2.07 (m, 8H), 2.59 (m, 1H), 3.45 (m, 1H), 3.52-3.78 (m, 8H), 6.57 (d, J=16 Hz, 1H), 6.63-6.86 (m, 2H), 7.17-7.27 (m, 2H), 7.41 (d, J=9 Hz, 1H), 7.80-7.89 (m, 1H); MS (ESI (+)) m/z 603.5 (M+H⁺). Trans: ¹H NMR (CDCl₃, 300 MHz) δ 1.26 (m, 2H), 1.56 (m, 2H), 2.15 (m, 4H), 2.35 (m, 1H), 3.25 (m, 1H), 3.57-3.78 (m, 8H), 6.57 (d, J=15 Hz, 1H), 6.80-6.99 (m, 2H), 7.24-7.32 (m, 2H), 7.41 (d, J=9 Hz, 1H), 7.80-7.89 (m, 1H); MS (ESI (+)) m/z 603.5 (M+H⁺).

### EXAMPLE 7

### Trans 4-{3-[4-(3-morpholin-4-yl-3-oxo-propenyl)-2,3-bis-trifluoromethyl-phenylsulfanyl]-phenylamino}-cyclohexanecarboxylic acid

The procedure of Example 6 was used to prepare the Trans isomer, which eluted on the HPLC as the minor product. Trans: ¹H NMR (CDCl₃, 300 MHz) δ 1.26 (m, 2H), 1.56 (m, 2H), 2.15 (m, 4H), 2.35 (m, 1H), 3.25 (m, 1H), 3.57-3.78 (m, 8H), 6.57 (d, J=15 Hz, 1H), 6.80-6.99 (m, 2H), 7.24-7.32 (m, 2H), 7.41 (d, J=9 Hz, 1H), 7.80-7.89 (m, 1H); MS (ESI (+)) m/z 603.5 (M+H⁺).

### EXAMPLE 8

### 3-[4-(3-Cyclobutylamino-phenylsulfanyl)-2,3-bis-trifluoromethyl-phenyl]-1-morpholin-4-yl-propenone

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 450 μL of dichloroethane and 19 μL of acetic acid was added. Cyclobutanone (11.6 μL, 0.16 mmol) then sodium triacetoxyborohydride (44 mg, 0.208 mmol) were added and the reaction was allowed to stir overnight. The crude reaction mixture was diluted with DMSO and purified by preparative HPLC as the trifluoroacetamide salt. ¹H NMR (DMSO-d₆, 300 MHz) δ1.65-1.85 (m, 4H), 2.26-2.35 (m, 2H), 3.53-3.71 (m, 8H), 3.82 (m, 1H), 6.59-6.65 (m, 2H), 6.68 (d, J=8 Hz, 1H), 7.17-7.23 (m, 2H), 7.68 (m, 1H), 8.03 (d, J=8 Hz, 1H); MS (ESI (+)) m/z 531.3 (M+H⁺).

### EXAMPLE 9

### (2-{3-[4-(3-Morpholin-4-yl-3-oxo-propenyl)-2,3-bis-trifluoromethyl-phenylsulfanyl]-phenylamino}-cyclopentyl)-acetic acid

The procedure from Example 6 was followed utilizing (2-oxo-cyclopentyl)-acetic acid ethyl ester as the starting ketone. MS (ESI (+)) m/z 603.4 (M+H⁺).

### EXAMPLE 10

### 3-[4-(3-Di(2-Methylene-cyclopropanecarboxylic acid)amino-phenylsulfanyl)-2,3-bis-trifluoromethyl-phenyl]-1-morpholin-4-yl-propenone

The procedure from Example 6 was followed utilizing 2-formyl-cyclopropanecarboxylic acid ethyl ester as the starting aldehyde. The reaction proceeded to give completely disubstituted product. The stereochemistry about the two cyclopropyl rings was primarily trans. The compound was submitted as a mixture of isomers about the cyclopropyl ring. MS (ESI (+)) m/z 673.5 (M+H⁺).

### EXAMPLE 11

### 3-(4-{3-[(3,5-Dimethyl-isoxazol-4-ylmethyl)-amino]-phenylsulfanyl}-2,3-bis-trifluoromethyl-phenyl)-1-morpholin-4-yl-propenone

The procedure from Example 8 was followed utilizing (3,5-dimethyl-isoxazol-4-yl)-acetaldehyde as the starting aldehyde. MS (ESI (+)) m/z 586.4 (M+H⁺).

### EXAMPLE 12

### 3-[4-(3-Benzylamino-phenylsulfanyl)-2,3-bis-trifluoromethyl-phenyl]-1-morpholin-4-yl-propenone

The procedure from Example 8 was followed utilizing benzaldehyde as the starting aldehyde. MS (ESI (+)) m/z 567.4 (M+H⁺).

### EXAMPLE 13

### Cis 3-{4-[3-(4-Methyl-cyclohexylamino)-phenylsulfanyl]-2,3-bis-trifluoromethyl-phenyl}-1-morpholin-4-yl-propenone

### EXAMPLE 14

### Trans 3-{4-[3-(4-Methyl-cyclohexylamino)-phenylsulfanyl]-2,3-bis-trifluoromethyl-phenyl}-1-morpholin-4-yl-propenone

The procedure from Example 8 was followed utilizing 4-methylcyclohexanone as the starting ketone. Both the cis and trans products were formed in this reaction. Both were isolated by preparative HPLC and submitted. The identity of each isomer was assigned based on the comparison of retention times and product distribution. Cis (ESI (+)) m/z 573.3 (M+H⁺), Trans (ESI (+)) m/z 573.5 (M+H⁺).

### EXAMPLE 15

### 1-Morpholin-4-yl-3-{4-[3-(tetrahydro-thiopyran-4-ylamino)-phenylsulfanyl]-2,3-bis-trifluoromethyl-phenyl}-propenone

The procedure from Example 8 was followed utilizing tetrahydro-4H-thiopyran-4-one as the starting ketone. MS (ESI (+)) m/z 577.4 (M+H⁺).

### EXAMPLE 16

### 3-{4-[3-(1,1-Dioxo-hexahydro-1λ⁶-thiopyran-4-ylamino)-phenylsulfanyl]-2,3-bis-trifluoromethyl-phenyl}-1-morpholin-4-yl-propenone

The procedure from Example 8 was followed utilizing 1,1-Dioxo-tetrahydro-1λ⁶-thiopyran-4-one as the starting ketone. The ketone was prepared as described in Rule et al. J Org Chem. 1995, 60:1665. MS (ESI (+)) m/z 609.3 (M+H⁺).

### EXAMPLE 17

### 1-Morpholin-4-yl-3-{4-[3-(tetrahydro-pyran-4-ylamino)-phenylsulfanyl]-2,3-bis-trifluoromethyl-phenyl}-propenone

The procedure from Example 8 was followed utilizing tetrahydro-4H-pyran-4-one as the starting ketone. MS (ESI (+)) m/z 561.3 (M+H⁺).

### EXAMPLE 18

### 3-{4-[3-(1-Methyl-piperidin-4-ylamino)-phenylsulfanyl]-2,3-bis-trifluoromethyl-phenyl}-1-morpholin-4-yl-propenone

The procedure of Example 2 was followed using methanesulfonic acid in place of boron trifluoride diethyl etherate. The resulting product was subjected to the procedures of Examples 3 and 4 to afford 3-[4-(3-amino-phenylsulfanyl)-2,3-bis-trifluoromethyl-phenyl]-1-morpholin-4-yl-propenone. The procedure from Example 8 was then followed utilizing 1-methyl-4-piperidone as the starting ketone. MS (ESI (+)) m/z 574.3 (M+H⁺).

### EXAMPLE 19

### 3-{4-[3-(1-Ethyl-piperidin-4-ylamino)-phenylsulfanyl]-2,3-bis-trifluoromethyl-phenyl}-1-morpholin-4-yl-propenone

The procedure from Example 8 was followed utilizing 1-ethyl-4-piperidone as the starting ketone. MS (ESI (+)) m/z 588.2 (M+H⁺).

### EXAMPLE 20

### 1-Morpholin-4-yl-3-{4-[3-(1-propyl-piperidin-4-ylamino)-phenylsulfanyl]-2,3-bis-trifluoromethyl-phenyl}-propenone

The procedure from Example 8 was followed utilizing 1-propyl-4-piperidone as the starting ketone. MS (ESI (+)) m/z 602.6 (M+H⁺).

### EXAMPLE 21

### 3-{4-[3-(1-Isopropyl-piperidin-4-ylamino)-phenylsulfanyl]-2,3-bis-trifluoromethyl-phenyl}-1-morpholin-4-yl-propenone

The procedure from Example 8 was followed utilizing 1-isopropyl-4-piperidone as the starting ketone. MS (ESI (+)) m/z 602.6 (M+H⁺).

### EXAMPLE 22

### 3-{4-[3-(8-Methyl-8-aza-bicyclo[3.2.1]oct-3-ylamino)-phenylsulfanyl]-2,3-bis-trifluoromethyl-phenyl}-1-morpholin-4-yl-propenone

The procedure from Example 8 was followed utilizing tropinone as the starting ketone. Two diastereomers were obtained. The major isomer was pure and was submitted while the minor isomer was impure and was not submitted. The stereochemistry of the major and minor isomers is not known at this time. MS (ESI (+)) m/z 600.5 (M+H⁺).

### EXAMPLE 23

### 3-{4-[3-(1-Acetyl-piperidin-4-ylamino)-phenylsulfanyl]-2,3-bis-trifluoromethyl-phenyl}-1-morpholin-4-yl-propenone

The procedure from Example 8 was followed utilizing 1-acetyl-4-piperidone as the starting ketone. MS (ESI (+)) m/z 602.4 (M+H⁺).

### EXAMPLE 24

### 4-{3-[4-(3-Morpholin-4-yl-3-oxo-propenyl)-2,3-bis-trifluoromethyl-phenylsulfanyl]-phenylamino}-piperidine-1-carboxylic acid ethyl ester

The procedure from Example 8 was followed utilizing N-carbethoxy-4-piperidone as the starting ketone. MS (ESI (+)) m/z 632.4 (M+H⁺).

### EXAMPLE 25

### 1-Morpholin-4-yl-3-{4-[3-(piperidin-4-ylamino)-phenylsulfanyl]-2,3-bis-trifluoromethyl-phenyl}-propenone

The procedure from Example 8 was followed utilizing N—BOC-4-piperidone as the starting ketone. The intermediate Boc protected piperidine was deprotected by addition to 1 mL of trifluoroacetic acid (TFA) (no solvent). HPLC analysis showed quantitative conversion to the product. The crude reaction was concentrated and dissolved in DMSO for purification by preparative HPLC. MS (ESI (+)) m/z 560.5 (M+H⁺).

### EXAMPLE 26

### 4-{3-[4-(3-Morpholin-4-yl-3-oxo-propenyl)-2,3-bis-trifluoromethyl-phenylsulfanyl]-phenylamino}-piperidine-1-carboxylic acid amide

The procedure from Example 8 was followed utilizing 4-oxo-piperidine-1-carboxylic acid amide as the starting ketone. MS (ESI (+)) m/z 603.6 (M+H⁺).

### EXAMPLE 27

### 1-Morpholin-4-yl-3-{4-[3-(piperidin-3-ylamino)-phenylsulfanyl]-2,3-bis-trifluoromethyl-phenyl}-propenone

The procedure from Example 8 was followed utilizing N—BOC-3-piperidone as the starting ketone. The intermediate Boc protected piperidine was deprotected by subjection to 1 mL of TFA (no solvent). HPLC analysis showed quantitative conversion to the product. The crude reaction was concentrated and dissolved in DMSO for purification by preparative HPLC. The compound was submitted as a racemic mixture. MS (ESI (+)) m/z 560.7 (M+H⁺).

### EXAMPLE 28

### 3-{4-[3-(1-Ethyl-piperidin-3-ylamino)-phenylsulfanyl]-2,3-bis-trifluoromethyl-phenyl}-1-morpholin-4-yl-propenone

The procedure from Example 8 was followed utilizing N-ethyl-3-piperidone as the starting ketone. The compound was submitted as a racemic mixture. MS (ESI (+)) m/z 588.5 (M+H⁺).

### EXAMPLE 29

### 3-{4-[3-(1-Aza-bicyclo[2.2.2]oct-3-ylamino)-phenylsulfanyl]-2,3-bis-trifluoromethyl-phenyl}-1-morpholin-4-yl-propenone

The procedure from Example 8 was followed utilizing 1-aza-bicyclo[2.2.2]octan-3-one as the starting ketone. The compound was submitted as a racemic mixture. MS (ESI (+)) m/z 586.6 (M+H⁺).

### EXAMPLE 30

### 3-{4-[3-(1-Benzyl-pyrrolidin-3-ylamino)-phenylsulfanyl]-2,3-bis-trifluoromethyl-phenyl}-1-morpholin-4-yl-propenone

The procedure from Example 8 was followed utilizing 1-benzyl-pyrrolidin-3-one as the starting ketone. MS (ESI (+)) m/z 636.7 (M+H⁺).

### EXAMPLE 31

### 3-{4-[3-(1-iso-butyl-piperidin-4-ylamino)-phenylsulfanyl]-2,3-bis-trifluoromethyl-phenyl}-1-morpholin-4-yl-propenone

The procedure from Example 8 was followed utilizing 1-iso-butyl-4-piperidone as the starting ketone. MS (ESI (+)) m/z 616.5 (M+H⁺).

### EXAMPLE 32

### 1-Morpholin-4-yl-3-{4-[3-(1,2,2,6,6-pentamethyl-piperidin-4-ylamino)-phenylsulfanyl]-2,3-bis-trifluoromethyl-phenyl}-propenone

The procedure from Example 8 was followed utilizing 1,2,2,6,6-pentamethyl-piperidin-4-one as the starting ketone. MS (ESI (+)) m/z 630.5 (M+H⁺).

### EXAMPLE 33

### Ethanesulfonic acid {3-[4-(3-morpholin-4-yl-3-oxo-propenyl)-2,3-bis-trifluoromethyl-phenylsulfanyl]-phenyl}-amide

The product of Example 4, 3-[4-(3-amino-phenylsulfanyl)-2,3-bis-trifluoromethyl-phenyl]-1-morpholin-4-yl-propenone (20 mg, 0.42 mmol), was dissolved in 180 μL DCM and 8 μL of pyridine was added. The reaction was cooled to 0° C. then ethane sulfonyl chloride (4.2 μL, 0.44 mmol) was added. The reaction was allowed to stir at 0° C. for 0.5 hr then at room temperature for an additional 0.5 hr. The crude reaction was diluted with DMSO and purified by preparative HPLC. ¹H NMR (CDCl₃, 300 MHz) δ 1.38 (t, J=7 Hz, 3H), 3.15 (q, J=7 Hz, 2H), 3.55-3.76 (m, 8H), 6.57 (d, J=15 Hz, 1H), 6.65 (m, 1H), 7.15-7.26 (m, 2H), 7.26-7.47 (m, 3H), 7.84 (m, 1H); MS (ESI (+)) m/z 569.3 (M+H⁺).

### EXAMPLE 34

### 2,2,2-Trifluoroethanesulfonic acid {3-[4-(3-morpholin-4-yl-3-oxo-propenyl)-2,3-bis-trifluoromethyl-phenylsulfanyl]-phenyl}-amide

The product of Example 4, 3-[4-(3-amino-phenylsulfanyl)-2,3-bis-trifluoromethyl-phenyl]-1-morpholin-4-yl-propenone (20 mg, 0.42 mmol), was dissolved in 180 μL DCM and 8 μL of pyridine was added. The reaction was cooled to 0° C. then trifluoroethane sulfonyl chloride (4.2 μL, 0.44 mmol) was added. The reaction was allowed to stir at 0° C. for 0.5 hr then at room temperature for an additional 0.5 hr. The crude reaction was diluted with DMSO and purified by preparative HPLC. ¹H NMR ((CD₃)₂CO, 300 MHz) δ 3.54-3.76 (m, 8H), 4.39 (q, J=10 Hz, 2H), 7.13 (d, J=16 Hz, 1H), 7.34 (m, 1H), 7.42-7.54 (m, 4H), 7.79-7.94 (m, 2H), 9.51 (s, 1H); MS (ESI (+)) m/z 623.3 (M+H⁺).

### EXAMPLE 35

### N-{3-[4-(3-Morpholin-4-yl-3-oxo-propenyl)-2,3-bis-trifluoromethyl-phenylsulfanyl]-phenyl}-methanesulfonamide

The procedure for Example 33 was run utilizing methane sulfonyl chloride as the starting sulfonyl chloride. MS (ESI (+)) m/z 555.1 (M+H⁺).

### EXAMPLE 36

### Propane-1-sulfonic acid {3-[4-(3-morpholin-4-yl-3-oxo-propenyl)-2,3-bis-trifluoromethyl-phenylsulfanyl]-phenyl}-amide

The procedure for Example 33 was run utilizing propane sulfonyl chloride as the starting sulfonyl chloride. MS (ESI (+)) m/z 583.3 (M+H⁺).

### EXAMPLE 37

### Butane-1-sulfonic acid {3-[4-(3-morpholin-4-yl-3-oxo-propenyl)-2,3-bis-trifluoromethyl-phenylsulfanyl]-phenyl}-amide

The procedure for Example 33 was run utilizing butane sulfonyl chloride as the starting sulfonyl chloride. MS (ESI (+)) m/z 597.5 (M+H⁺).

### EXAMPLE 38

### N-{3-[4-(3-Morpholin-4-yl-3-oxo-propenyl)-2,3-bis-trifluoromethyl-phenylsulfanyl]-phenyl}-C-pyridin-4-yl-methanesulfonamide

The procedure for Example 33 was run utilizing 4-pyridylmethyl sulfonyl chloride as the starting sulfonyl chloride. MS (ESI (+)) m/z 632.2 (M+H⁺).

### EXAMPLE 39

### N-{3-[4-(3-Morpholin-4-yl-3-oxo-propenyl)-2,3-bis-trifluoromethyl-phenylsulfanyl]-phenyl}-C-pyridin-2-yl-methanesulfonamide

The procedure for Example 33 was run utilizing 2-pyridylmethyl sulfonyl chloride as the starting sulfonyl chloride. MS (ESI (+)) m/z 632.3 (M+H⁺).

### EXAMPLE 40

### N-{3-[4-(3-Morpholin-4-yl-3-oxo-propenyl)-2,3-bis-trifluoromethyl-phenylsulfanyl]-phenyl}-C-pyridin-3-yl-methanesulfonamide

The procedure for Example 33 was run utilizing 3-pyridylmethyl sulfonyl chloride as the starting sulfonyl chloride. MS (ESI (+)) m/z 632.3 (M+H⁺).

### EXAMPLE 41

### N-{3-[4-(3-Morpholin-4-yl-3-oxo-propenyl)-2,3-bis-trifluoromethyl-phenylsulfanyl]-phenyl}-benzenesulfonamide

The procedure for Example 33 was run utilizing benzene sulfonyl chloride as the starting sulfonyl chloride. MS (ESI (+)) m/z 617.2 (M+H⁺).

### EXAMPLE 42

### 2-Fluoro-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 2-fluorobenzene sulfonyl chloride as the starting sulfonyl chloride. MS (ESI (+)) m/z 635.2 (M+H⁺).

### EXAMPLE 43

### 3-Fluoro-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 3-fluorobenzene sulfonyl chloride as the starting sulfonyl chloride. MS (ESI (+)) m/z 635.2 (M+H⁺).

### EXAMPLE 44

### 4-Fluoro-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 4-fluorobenzene sulfonyl chloride as the starting sulfonyl chloride MS (ESI (+)) m/z 635.3 (M+H⁺).

### EXAMPLE 45

### 4-methyl-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 4-methylbenzene sulfonyl chloride as the starting sulfonyl chloride. MS (ESI (+)) m/z 631.3 (M+H⁺).

### EXAMPLE 46

### 3-methyl-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 3-methylbenzene sulfonyl chloride as the starting sulfonyl chloride. MS (ESI (+)) m/z 631.3 (M+H⁺).

### EXAMPLE 47

### 2-Chloro-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 2-chlorobenzene sulfonyl chloride as the starting sulfonyl chloride. MS (ESI (+)) m/z 651.0 (M+H⁺)

### EXAMPLE 48

### 3-Chloro-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 3-chlorobenzene sulfonyl chloride as the starting sulfonyl chloride. MS (ESI (+)) m/z 651.0 (M+H⁺).

### EXAMPLE 49

### 4-Chloro-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 4-chlorobenzene sulfonyl chloride as the starting sulfonyl chloride. MS (ESI (+)) m/z 651.0 (M+H⁺).

### EXAMPLE 50

### 4-methoxy-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 4-methoxybenzene sulfonyl chloride as the starting sulfonyl chloride. MS (ESI (+)) m/z 647.3 (M+H⁺).

### EXAMPLE 51

### N-{3-[4-(3-Morpholin-4-yl-3-oxo-propenyl)-2,3-bis-trifluoromethyl-phenylsulfanyl]-phenyl}-2-nitro-benzenesulfonamide

The procedure for Example 33 was run utilizing 2-nitrobenzene sulfonyl chloride as the starting sulfonyl chloride. MS (ESI (+)) m/z 662.1 (M+H⁺).

### EXAMPLE 52

### N-{3-[4-(3-Morpholin-4-yl-3-oxo-propenyl)-2,3-bis-trifluoromethyl-phenylsulfanyl]-phenyl}-3-nitro-benzenesulfonamide

The procedure for Example 33 was run utilizing 3-nitrobenzene sulfonyl chloride as the starting sulfonyl chloride. MS (ESI (+)) m/z 662.1 (M+H⁺).

### EXAMPLE 53

### N-{3-[4-(3-Morpholin-4-yl-3-oxo-propenyl)-2,3-bis-trifluoromethyl-phenylsulfanyl]-phenyl}-4-nitro-benzenesulfonamide

The procedure for Example 33 was run utilizing 4-nitrobenzene sulfonyl chloride as the starting sulfonyl chloride. MS (ESI (+)) m/z 662.1 (M+H⁺).

### EXAMPLE 54

### 3-methoxy-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 3-methoxybenzene sulfonyl chloride as the starting sulfonyl chloride. MS (ESI (+)) m/z 647.3 (M+H⁺).

### EXAMPLE 55

### N-{3-[4-(3-Morpholin-4-yl-3-oxo-propenyl)-2,3-bis-trifluoromethyl-phenylsulfanyl]-phenyl}-C-phenyl-methanesulfonamide

The procedure for Example 33 was run utilizing benzyl sulfonyl chloride as the starting sulfonyl chloride. MS (ESI (+)) m/z 631.2 (M+H⁺).

### EXAMPLE 56

### 5-Methyl-isoxazole-3-sulfonic acid {3-[4-(3-morpholin-4-yl-3-oxo-propenyl)-2,3-bis-trifluoromethyl-phenylsulfanyl]-phenyl}-amide

The procedure for Example 33 was run utilizing 5-methyl-isoxazole-3-sulfonyl chloride as the starting sulfonyl chloride. MS (ESI (+)) m/z 622.2 (M+H⁺).

### EXAMPLE 57

### Thiophene-2-sulfonic acid {3-[4-(3-morpholin-4-yl-3-oxo-propenyl)-2,3-bis-trifluoromethyl-phenylsulfanyl]-phenyl}-amide

The procedure for Example 33 was run utilizing thiophene-2-sulfonyl chloride as the starting sulfonyl chloride. MS (ESI (+)) m/z 622.9 (M+H⁺).

### EXAMPLE 58

### Thiophene-3-sulfonic acid {3-[4-(3-morpholin-4-yl-3-oxo-propenyl)-2,3-bis-trifluoromethyl-phenylsulfanyl]-phenyl}-amide

The procedure for Example 33 was run utilizing thiophene-3-sulfonyl chloride as the starting sulfonyl chloride. MS (ESI (+)) m/z 623.1 (M+H⁺).

### EXAMPLE 59

### C-Methanesulfonyl-N-{3-[4-(3-morpholin-4-yl-3-oxo-propenyl)-2,3-bis-trifluoromethyl-phenylsulfanyl]-phenyl}-methanesulfonamide

The procedure for Example 33 was run utilizing methylsulfomethanesulfonyl chloride as the starting sulfonyl chloride. MS (ESI (+)) m/z 633.0 (M+H⁺).

### EXAMPLE 60

### 2,6-Dichloro-N-{3-[4-(3-morpholin-4-yl-3-oxo-propenyl)-2,3-bis-trifluoromethyl-phenylsulfanyl]-phenyl}-benzenesulfonamide

The procedure for Example 33 was run utilizing 2,6-dichlorobenzene sulfonyl chloride as the starting sulfonyl chloride. MS (ESI (+)) m/z 684.9 (M+H⁺).

### EXAMPLE 61

### Amino sulfonic acid {3-[4-(3-morpholin-4-yl-3-oxo-propenyl)-2,3-bis-trifluoromethyl-phenylsulfanyl]-phenyl}-amide

The procedure for Example 33 was run utilizing amino sulfonyl chloride as the starting sulfonyl chloride. MS (ESI (+)) m/z 556.1 (M+H⁺).

### EXAMPLE 62

### Dimethyl amino sulfonic acid {3-[4-(3-morpholin-4-yl-3-oxo-propenyl)-2,3-bis-trifluoromethyl-phenylsulfanyl]-phenyl}-amide

The procedure for Example 33 was run utilizing dimethyl amino sulfonyl chloride as the starting sulfonyl chloride. MS (ESI (+)) m/z 584.1 (M+H⁺).

### EXAMPLE 63

### 1-Isopropyl-3-{3-[4-(3-morpholin-4-yl-3-oxo-propenyl)-2,3-bis-trifluoromethyl-phenylsulfanyl]-phenyl}-urea

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 225 μL THF and isopropyl isocyanate (7.67 μL, 0.078 mmol) and triethylamine (9.3 μL, 0.068 mmol) were added. HPLC analysis after stirring overnight showed quantitative formation of the product. The crude reaction was diluted with DMSO and purified by preparative HPLC. ¹H NMR (DMSO-d₆, 300 MHz) δ 1.08 (d, J=7 Hz, 6H), 3.54-3.78 (m, 9H), 6.07 (d, J=8 Hz, 1H), 7.07 (d, J=8 Hz, 1H), 7.19 (d, J=16 Hz, 1H), 7.31 (d, J=8 Hz, 1H), 7.35 (t, J=8 Hz, 1H), 7.42 (d, 8 Hz, 1H), 7.63-7.71 (m, 2H), 8.01 (d, J=8 Hz, 1H), 8.53 (s, 1H); MS (ESI (+)) m/z 562.3 (M+H⁺).

### EXAMPLE 64

### 1-Methyl-3-{3-[4-(3-morpholin-4-yl-3-oxo-propenyl)-2,3-bis-trifluoromethyl-phenylsulfanyl]-phenyl}-urea

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 225 μL THF and methyl isocyanate (5.93 μL, 0.104 mmol) was added. HPLC analysis after stirring o/n showed quantitative formation of the product. The crude reaction was diluted with DMSO and purified by preparative HPLC. ¹H NMR (DMSO-d₆, 300 MHz) δ 2.62 (d, J=5 Hz, 3H), 3.53-3.70 (m, 8H), 6.09 (d, J=5 Hz, 1H), 7.07 (d, J=7 Hz, 1H), 7.20 (d, J=15 Hz, 1H), 7.31 (d, J=8 Hz, 1H), 7.35 (t, J=8 Hz, 1H), 7.47 (d, J=8 Hz, 1H), 7.63-7.71 (m, 2H), 8.02 (d, J=8 Hz, 1H), 8.75 (s, 1H); MS (ESI (+)) m/z 534.1 (M+H⁺).

### EXAMPLE 65

### 1-Ethyl-3-{3-[4-(3-morpholin-4-yl-3-oxo-propenyl)-2,3-bis-trifluoromethyl-phenylsulfanyl]-phenyl}-urea

The procedure for Example 63 was followed utilizing ethyl isocyanate as the starting isocyanate. MS (ESI (+)) m/z 548.3 (M+H⁺).

### EXAMPLE 66

### 1-{3-[4-(3-Morpholin-4-yl-3-oxo-propenyl)-2,3-bis-trifluoromethyl-phenylsulfanyl]-phenyl}-3-propyl-urea

The procedure for Example 63 was followed utilizing propyl isocyanate as the starting isocyanate. MS (ESI (+)) m/z 562.5 (M+H⁺).

### EXAMPLE 67

### 1-Butyl-3-{3-[4-(3-morpholin-4-yl-3-oxo-propenyl)-2,3-bis-trifluoromethyl-phenylsulfanyl]-phenyl}-urea

The procedure for Example 64 was followed utilizing butyl isocyanate as the starting isocyanate. MS (ESI (+)) m/z 576.5 (M+H⁺).

### EXAMPLE 68

### 1-Cyclopentyl-3-{3-[4-(3-morpholin-4-yl-3-oxo-propenyl)-2,3-bis-trifluoromethyl-phenylsulfanyl]-phenyl}-urea

The procedure for Example 64 was followed utilizing cyclopentyl isocyanate as the starting isocyanate. MS (ESI (+)) m/z 588.4 (M+H⁺).

### EXAMPLE 69

### 1-{3-[4-(3-Morpholin-4-yl-3-oxo-propenyl)-2,3-bis-trifluoromethyl-phenylsulfanyl]-phenyl}-3-phenyl-urea

The procedure for Example 64 was followed utilizing phenyl isocyanate as the starting isocyanate. MS (ESI (+)) m/z 596.2 (M+H⁺).

### EXAMPLE 70

### 1-Benzyl-3-{3-[4-(3-morpholin-4-yl-3-oxo-propenyl)-2,3-bis-trifluoromethyl-phenylsulfanyl]-phenyl}-urea

The procedure for Example 64 was followed utilizing benzyl isocyanate as the starting isocyanate. MS (ESI (+)) m/z 610.5 (M+H⁺).

### EXAMPLE 71

### 1-{3-[4-(3-Morpholin-4-yl-3-oxo-propenyl)-2,3-bis-trifluoromethyl-phenylsulfanyl]-phenyl}-3-(2-thiophen-2-yl-ethyl)-urea

The procedure for Example 64 was followed utilizing 2-(2-isocyanato-ethyl)-thiophene as the starting isocyanate. MS (ESI (+)) m/z 630.4 (M+H⁺).

### EXAMPLE 72

### (3-{3-[4-(3-Morpholin-4-yl-3-oxo-propenyl)-2,3-bis-trifluoromethyl-phenylsulfanyl]-phenyl}-ureido)-acetic acid

The procedure for Example 64 was followed utilizing ethyl isocyanatoacetate as the starting isocyanate. The purified product was then hydrolyzed in 2:1 THF/H₂O by adding 2N LiOH until basic. The crude was then concentrated and diluted in DMSO for preparative HPLC purification. MS (ESI (+)) m/z 578.3 (M+H⁺).

### EXAMPLE 73

### 3-(3-{3-[4-(3-Morpholin-4-yl-3-oxo-propenyl)-2,3-bis-trifluoromethyl-phenylsulfanyl]-phenyl}-ureido)-propionic acid

The procedure for Example 64 was followed utilizing 3-isocyanatopropionic acid as the starting isocyanate. The purified product was then hydrolyzed in 2:1 THF/H₂O by adding 2N LiOH until basic. The crude was then concentrated and diluted in DMSO for preparative HPLC purification. MS (ESI (+)) m/z 592.3 (M+H⁺).

### EXAMPLE 74

### 4-(3-{3-[4-(3-Morpholin-4-yl-3-oxo-propenyl)-2,3-bis-trifluoromethyl-phenylsulfanyl]-phenyl}-ureido)-butyric acid

The procedure for Example 64 was followed utilizing 4-isocyanatobutyric acid as the starting isocyanate. The purified product was then hydrolyzed in 2:1 THF/H₂O by adding 2N LiOH until basic. The crude was then concentrated and diluted in DMSO for preparative HPLC purification. MS (ESI (+)) m/z 606.3 (M+H⁺).

### EXAMPLE 75

### Morpholine-4-carboxylic acid {3-[4-(3-morpholin-4-yl-3-oxo-propenyl)-2,3-bis-trifluoromethyl-phenylsulfanyl]-phenyl}-amide

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 400 μL methylene chloride and 4-morpholinylcarbonyl chloride (9.12 μL, 0.078 mmol) was added. The reaction stirred at room temperature over the weekend to give 60% conversion. The crude was then diluted in DMSO and purified by preparative HPLC. ¹H NMR ((CD₃)₂CO, 300 MHz) δ 3.57-3.79 (m, 16H), 7.08-7.20 (m, 2H), 7.31-7.43 (m, 3H), 7.65-7.91 (m, 5H), 8.05-8.18 (s, 1H); MS (ESI (+)) m/z 590.7 (M+H⁺).

### EXAMPLE 76

### 1-(2-Hydroxy-ethyl)-3-{3-[4-(3-morpholin-4-yl-3-oxo-propenyl)-2,3-bis-trifluoromethyl-phenylsulfanyl]-phenyl}-urea

The procedure for Example 64 was followed utilizing 2-methyl-acrylic acid 2-isocyanato-ethyl ester as the starting isocyanate. The purified product was then hydrolyzed in 2:1 THF/H₂O by adding 2N LiOH until basic. The crude was then concentrated and diluted in DMSO for preparative HPLC purification. MS (ESI (+)) m/z 564.2 (M+H⁺).

### EXAMPLE 77

### 1-Methyl-3-{3-[4-(3-morpholin-4-yl-3-oxo-propenyl)-2,3-bis-trifluoromethyl-phenylsulfanyl]-phenyl}-thiourea

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 250 μL THF and methyl isothiocyanate (22.8 μl, 0.312 mmol) was added. HPLC analysis after stirring o/n showed quantitative formation of the product. The crude reaction was diluted with DMSO and purified by preparative HPLC. MS (ESI (+)) m/z 550.2 (M+H⁺).

### EXAMPLE 78

### 1-Ethyl-3-{3-[4-(3-morpholin-4-yl-3-oxo-propenyl)-2,3-bis-trifluoromethyl-phenylsulfanyl]-phenyl}-thiourea

The procedure for Example 77 was followed utilizing ethyl isothiocyanate as the starting isothiocyanate. MS (ESI (+)) m/z 564.2 (M+H⁺).

### EXAMPLE 79

### 1-{3-[4-(3-Morpholin-4-yl-3-oxo-propenyl)-2,3-bis-trifluoromethyl-phenylsulfanyl]-phenyl}-3-propyl-thiourea

The procedure for Example 77 was followed utilizing propyl isothiocyanate as the starting isothiocyanate. MS (ESI (+)) m/z 577.7 (M+H⁺).

### EXAMPLE 80

### 1-Butyl-3-{3-[4-(3-morpholin-4-yl-3-oxo-propenyl)-2,3-bis-trifluoromethyl-phenylsulfanyl]-phenyl}-thiourea

The procedure for Example 77 was followed utilizing butyl isothiocyanate as the starting isothiocyanate. MS (ESI (+)) m/z 592.2 (M+H⁺).

### EXAMPLE 81

### 1-{3-[4-(3-Morpholin-4-yl-3-oxo-propenyl)-2,3-bis-trifluoromethyl-phenylsulfanyl]-phenyl}-3-phenyl-thiourea

The procedure for Example 77 was followed utilizing phenyl isothiocyanate as the starting isothiocyanate. MS (ESI (+)) m/z 612.3 (M+H⁺).

### EXAMPLE 82

### 1-Benzyl-3-{3-[4-(3-morpholin-4-yl-3-oxo-propenyl)-2,3-bis-trifluoromethyl-phenylsulfanyl]-phenyl}-thiourea

The procedure for Example 77 was followed utilizing benzyl isothiocyanate as the starting isothiocyanate. MS (ESI (+)) m/z 626.3 (M+H⁺).

### EXAMPLE 83

### 1-(2-Methoxy-ethyl)-3-{3-[4-(3-morpholin-4-yl-3-oxo-propenyl)-2,3-bis-trifluoromethyl-phenylsulfanyl]-phenyl}-thiourea

The procedure for Example 77 was followed utilizing methoxyethyl isothiocyanate as the starting isothiocyanate. MS (ESI (+)) m/z 593.5 (M+H⁺).

### EXAMPLE 84

### 3-(3-{3-[4-(3-Morpholin-4-yl-3-oxo-propenyl)-2,3-bis-trifluoromethyl-phenylsulfanyl]-phenyl}-thioureido)-propionic acid methyl ester

The procedure for Example 77 was followed utilizing 3-isothiocyanatopropionic acid methyl ester as the starting isothiocyanate. MS (ESI (+)) m/z 622.1 (M+H⁺).

### EXAMPLE 85

### {3-[4-(3-Morpholin-4-yl-3-oxo-propenyl)-2,3-bis-trifluoromethyl-phenylsulfanyl]-phenyl}-carbamic acid methyl ester

The product of Example 4, 3-[4-(3-amino-phenylsulfanyl)-2,3-bis-trifluoromethyl-phenyl]-1-morpholin-4-yl-propenone (20 mg, 0.042 mmol), was dissolved in 200 μL methylene chloride then pyridine (17 μL, 0.21 mmol) and methyl chloroformate (3.6 μL, 0.046 mmol) were added. HPLC analysis after stirring for one hour at room temperature showed formation of the product quantitatively. The crude reaction was diluted in DMSO and purified by preparative HPLC. ¹H NMR (DMSO-d₆, 400 MHz) δ 3.53-3.72 (m, 11H), 7.18 (d, J=8 Hz, 1H), 7.22 (d, J=16 Hz, 1H), 7.35 (d, J=8 Hz, 1H), 7.44 (t, J=8 Hz, 1H), 7.58 (d, J=8 Hz, 1H), 7.64-7.73 (m, 2H), 8.04 (d, J=8 Hz, 1H), 9.87 (s, 1H); MS (ESI (+)) m/z 535.3 (M+H⁺).

### EXAMPLE 86

### {3-[4-(3-Morpholin-4-yl-3-oxo-propenyl)-2,3-bis-trifluoromethyl-phenylsulfanyl]-phenyl}-carbamic acid ethyl ester

The product of Example 4, 3-[4-(3-amino-phenylsulfanyl)-2,3-bis-trifluoromethyl-phenyl]-1-morpholin-4-yl-propenone (20 mg, 0.042 mmol), was dissolved in 200 μL methylene chloride then pyridine (17 μL, 0.21 mmol) and ethyl chloroformate (8.1 μL, 0.084 mmol) were added. HPLC analysis after stirring for one hour at room temperature showed formation of the product quantitatively. The crude reaction was diluted in DMSO and purified by preparative HPLC. ¹H NMR (DMSO-d₆, 400 MHz) δ 1.23 (t, J=7 Hz, 3H), 3.53-3.70 (m, 8H), 4.12 (q, J=7 Hz, 2H), 7.16 (d, J=8 Hz, 1H), 7.21 (d, J=16 Hz, 1H), 7.33 (d, J=8 Hz, 1H), 7.41 (t, J=8 Hz, 1H), 7.56 (d, J=8 Hz, 1H), 7.63-7.72 (m, 2H), 8.03 (d, J=8 Hz, 1H), 9.83 (s, 1H); MS (ESI (+)) m/z 549.3 (M+H+).

### EXAMPLE 87

### {3-[4-(3-Morpholin-4-yl-3-oxo-propenyl)-2,3-bis-trifluoromethyl-phenylsulfanyl]-phenyl}-carbamic acid propyl ester

The procedure for Example 86 was followed utilizing propyl chloroformate as the starting chloroformate. MS (ESI (+)) m/z 563.2 (M+H+).

### EXAMPLE 88

### {3-[4-(3-Morpholin-4-yl-3-oxo-propenyl)-2,3-bis-trifluoromethyl-phenylsulfanyl]-phenyl}-carbamic acid butyl ester

The procedure for Example 86 was followed utilizing butyl chloroformate as the starting chloroformate. MS (ESI (+)) m/z 577.3 (M+H+).

### EXAMPLE 89

### {3-[4-(3-Morpholin-4-yl-3-oxo-propenyl)-2,3-bis-trifluoromethyl-phenylsulfanyl]-phenyl}-carbamic acid isopropyl ester

The procedure for Example 86 was followed utilizing isopropyl chloroformate as the starting chloroformate. MS (ESI (+)) m/z 563.2 (M+H+).

### EXAMPLE 90

### {3-[4-(3-Morpholin-4-yl-3-oxo-propenyl)-2,3-bis-trifluoromethyl-phenylsulfanyl]-phenyl}-carbamic acid phenyl ester

The procedure for Example 86 was followed utilizing benzene chloroformate as the starting chloroformate. MS (ESI (+)) m/z 597.3 (M+H+).

### EXAMPLE 91

### {3-[4-(3-Morpholin-4-yl-3-oxo-propenyl)-2,3-bis-trifluoromethyl-phenylsulfanyl]-phenyl}-carbamic acid benzyl ester

The procedure for Example 86 was followed utilizing benzyl chloroformate as the starting chloroformate. MS (ESI (+)) m/z 611.3 (M+H+).

### EXAMPLE 92

### Cis 4-({3-[4-(3-morpholin-4-yl-3-oxo-propenyl)-2,3-bis-trifluoromethyl-phenylsulfanyl]-phenylamino}-methyl)-cyclohexanecarboxylic acid

The product of Example 51, N-{3-[4-(3-morpholin-4-yl-3-oxo-propenyl)-2,3-bis-trifluoromethyl-phenylsulfanyl]-phenyl}-2-nitro-benzenesulfonamide (101 mg, 0.15 mmol), triphenyl phosphine (101 mg, 0.39 mmol), and cis-4-hydroxymethyl-cyclohexanecarboxylic acid methyl ester (97 mg, 0.56 mmol) were dissolved in 1.5 mL THF. Diisopropylazodicarboxylate (DIAD) (60 μL, 0.31 mmol) was then added and the reaction was stirred for 3 days at room temperature. The crude reaction mixture was concentrated then dissolved in ethyl acetate. The ethyl acetate washed once with brine and the organic layer was dried with sodium sulfate, filtered, and evaporated. The reaction was purified by flash chromatography using a gradient from 1:1 to 1:3 hexanes:ethyl acetate (57 mg, 47%).

The nosyl group was then deprotected by dissolving the product from the previous step (57 mg, 0.07 mmol) in 3 mL of DMF and adding potassium carbonate (104 mg, 0.75 mmol), phenyl sulfide (22 μL, 0.21 mmol). After 30 minutes at room temperature the product was formed quantitatively. The crude was dissolved in ethyl acetate then extracted with brine. The organic layer was then dried with sodium sulfate, filtered, and concentrated. The crude was then purified by flash chromatography using a gradient from 1:1 to 1:2 hexanes:ethyl acetate (38 mg, 86%).

Deprotection of the methyl ester was then performed by dissolving the product (38 mg, 0.060) in 6 mL of 1:1 THF:MeOH and adding 3 mL of 2N LiOH. After 30 minutes the ester was hydrolyzed and the crude was evaporated to dryness. The crude was dissolved in ethyl acetate and washed once with brine before drying with sodium sulfate, filtration, and concentration. The concentrated crude was dissolved in DMSO and purified by preparative HPLC to give the pure product (27 mg, 72%). ¹H NMR (DMSO-d₆, 300 MHz) δ 1.22 (m, 2H), 1.43-1.68 (m, 5H), 1.91 (m, 2H), 2.91 (m, 1H), 2.78 (s, 2H), 3.53-3.72 (m, 8H), 6.65-6.77 (m, 3H), 7.17-7.28 (m, 2H), 7.37 (d, J=8 Hz, 1H), 7.71 (m, 1H), 7.99-8.11 (m, 2H); MS (ESI (+)) m/z 617.5 (M+H+).

### EXAMPLE 93

### Trans 4-({3-[4-(3-morpholin-4-yl-3-oxo-propenyl)-2,3-bis-trifluoromethyl-phenylsulfanyl]-phenylamino}-methyl)-cyclohexanecarboxylic acid

The product of Example 51, N-{3-[4-(3-morpholin-4-yl-3-oxo-propenyl)-2,3-bis-trifluoromethyl-phenylsulfanyl]-phenyl}-2-nitro-benzenesulfonamide (99 mg, 0.15 mmol), triphenyl phosphine (104 mg, 0.40 mmol), and trans-4-hydroxymethyl-cyclohexanecarboxylic acid methyl ester (106 mg, 0.62 mmol) were dissolved in 1.5 mL THF. DIAD (60 μL, 0.31 mmol) was then added and the reaction was stirred for 3 days at room temperature. The crude reaction mixture was concentrated then dissolved in ethyl acetate. The ethyl acetate washed once with brine and the organic layer was dried with sodium sulfate, filtered, and evaporated. The reaction was purified by flash chromatography using a gradient from 1:1 to 1:3 hexanes:ethyl acetate (82 mg, 67%).

The nosyl group was then deprotected by dissolving the product from the previous step (82 mg, 0.10 mmol) in 3 mL of DMF and adding potassium carbonate (110 mg, 0.80 mmol), phenyl sulfide (31 μL, 0.3 mmol). After 30 minutes at room temperature the product was formed quantitatively. The crude was dissolved in ethyl acetate then extracted with brine. The organic layer was then dried with sodium sulfate, filtered, and concentrated. The crude was then purified by flash chromatography using a gradient from 1:1 to 1:2 hexanes:ethyl acetate (55 mg, 87%).

Deprotection of the methyl ester was then performed by dissolving the product (55 mg, 0.087) in 6 mL of 1:1 THF:MeOH and adding 3 mL of 2N LiOH. After 30 minutes the ester was hydrolyzed and the crude was evaporated to dryness. The crude was dissolved in ethyl acetate and washed once with brine before drying with sodium sulfate, filtration, and concentration. The concentrated crude was dissolved in DMSO and purified by preparative HPLC to give the pure product (50 mg, 93%). ¹H NMR (DMSO-d₆, 300 MHz) δ 0.98 (m, 2H), 1.20-1.38 (m, 2H), 1.47 (br, 1H), 1.88 (m, 4H), 2.14 (m, 1H), 2.85 (t, J=6 Hz, 2H), 3.53-3.72 (m, 8H), 6.01 (t, J=5 Hz, 1H), 6.63-6.71 (m, 3H), 7.15-7.23 (m, 2H), 7.34 (d, J=8 Hz, 1H), 7.68 (m, 1H), 8.03 (d, J=8 Hz, 1H), 12.00 (s, 1H); MS (ESI (+)) m/z 617.4 (M+H+).

### EXAMPLE 94

### Cis 3-({3-[4-(3-morpholin-4-yl-3-oxo-propenyl)-2,3-bis-trifluoromethyl-phenylsulfanyl]-phenylamino}-methyl)-cyclohexanecarboxylic acid

The procedure for Example 93 was followed utilizing methyl cis 3-hydroxymethyl-cyclohexanecarboxylic acid as the starting alcohol. MS (ESI (+)) m/z 617.4 (M+H+).

### EXAMPLE 95

### N-{3-[4-(3-Morpholin-4-yl-3-oxo-propenyl)-2,3-bis-trifluoromethyl-phenylsulfanyl]-phenyl}-isonicotinamide

Isonicotinic acid (7.63 mg, 0.062 mmol) and diisopropyl ethylamine (36 μL, 0.21) were dissolved in 500 μL of DMF. HATU (25.7 mg, 0.067 mmol) was then added and the reaction was allowed to stir for a couple of minutes at room temperature. The product of Example 4, 3-[4-(3-amino-phenylsulfanyl)-2,3-bis-trifluoromethyl-phenyl]-1-morpholin-4-yl-propenone (25 mg, 0.052 mmol), was then added and the reaction was allowed to stir overnight. HPLC analysis showed quantitative conversion of the starting material to product. The crude was diluted in DMSO and purified by preparative HPLC. ¹H NMR ((CD₃)₂CO, 300 MHz) δ 3.55-3.77 (m, 8H), 7.15 (d, J=16 Hz, 1H), 7.36 (d, J=8 Hz, 1H), 7.44 (d, J=8 Hz, 1H), 7.54 (t, J=8 Hz, 1H), 7.78-8.02 (m, 5H), 8.08 (s, 1H), 8.83 (d, J=6 Hz, 2H); MS (ESI (+)) m/z 582.3 (M+H+).

### EXAMPLE 96

### N-{3-[4-(3-Morpholin-4-yl-3-oxo-propenyl)-2,3-bis-trifluoromethyl-phenylsulfanyl]-phenyl}-2-(1H-tetrazol-5-yl)-acetamide

(1H-Tetrazol-5-yl)-acetic acid (7.94 mg, 0.062 mmol) and diisopropyl ethylamine (36 μL, 0.21) were dissolved in 500 μL of DMF. HATU (25.7 mg, 0.067 mmol) was then added and the reaction was allowed to stir for a couple of minutes at room temperature. The product of Example 4, 3-[4-(3-amino-phenylsulfanyl)-2,3-bis-trifluoromethyl-phenyl]-1-morpholin-4-yl-propenone (25 mg, 0.052 mmol), was then added and the reaction was allowed to stir overnight. HPLC analysis showed quantitative conversion of the starting material to product. The crude was diluted in DMSO and purified by preparative HPLC. ¹H NMR ((CD₃)₂CO, 300 MHz) δ 3.59-3.76 (m, 8H), 4.24 (s, 2H), 7.14 (d, J=16 Hz, 1H), 7.29 (d, J=8 Hz, 1H), 7.39 (d, J=8 Hz, 1H), 7.47 (t, J=8 Hz, 1H), 7.73 (d, J=8 Hz, 1H), 7.78-7.94 (m, 3H), 9.95 (s, 1H); MS (ESI (+)) m/z 587.4 (M+H+).

### EXAMPLE 97

### 2-Methoxy-N-{3-[4-(3-morpholin-4-yl-3-oxo-propenyl)-2,3-bis-trifluoromethyl-phenylsulfanyl]-phenyl}-acetamide

The procedure for Example 95 was followed utilizing methoxy-acetic acid as the starting carboxylic acid. MS (ESI (+)) m/z 549.0 (M+H+).

### EXAMPLE 98

### Pyridine-2-carboxylic acid {3-[4-(3-morpholin-4-yl-3-oxo-propenyl)-2,3-bis-trifluoromethyl-phenylsulfanyl]-phenyl}-amide

The procedure for Example 95 was followed utilizing pyridine-2-carboxylic acid as the starting carboxylic acid. MS (ESI (+)) m/z 582.5 (M+H+).

### EXAMPLE 99

### Pyridine-3-carboxylic acid {3-[4-(3-morpholin-4-yl-3-oxo-propenyl)-2,3-bis-trifluoromethyl-phenylsulfanyl]-phenyl}-amide

The procedure for Example 95 was followed utilizing pyridine-3-carboxylic acid as the starting carboxylic acid. MS (ESI (+)) m/z 582.4 (M+H+).

### EXAMPLE 100

### 2-Dimethylamino-N-{3-[4-(3-morpholin-4-yl-3-oxo-propenyl)-2,3-bis-trifluoromethyl-phenylsulfanyl]-phenyl}-acetamide

The procedure for Example 95 was followed utilizing dimethylamino-acetic acid as the starting carboxylic acid. MS (ESI (+)) m/z 562.4 (M+H+).

### EXAMPLE 101

### Isoxazole-5-carboxylic acid {3-[4-(3-morpholin-4-yl-3-oxo-propenyl)-2,3-bis-trifluoromethyl-phenylsulfanyl]-phenyl}-amide

The procedure for Example 95 was followed utilizing isoxazole-5-carboxylic acid as the starting carboxylic acid. MS (ESI (+)) m/z 572.5 (M+H+).

### EXAMPLE 102

### N-{3-[4-(3-Morpholin-4-yl-3-oxo-propenyl)-2,3-bis-trifluoromethyl-phenylsulfanyl]-phenyl}-2-pyridin-2-yl-acetamide

The procedure for Example 95 was followed utilizing 2-pyridyl acetic acid as the starting carboxylic acid. MS (ESI (+)) m/z 596.3 (M+H+).

### EXAMPLE 103

### N-{3-[4-(3-Morpholin-4-yl-3-oxo-propenyl)-2,3-bis-trifluoromethyl-phenylsulfanyl]-phenyl}-2-pyridin-3-yl-acetamide

The procedure for Example 95 was followed utilizing 3-pyridyl acetic acid as the starting carboxylic acid. MS (ESI (+)) m/z 596.4 (M+H+).

### EXAMPLE 104

### N-{3-[4-(3-Morpholin-4-yl-3-oxo-propenyl)-2,3-bis-trifluoromethyl-phenylsulfanyl]-phenyl}-2-pyridin-3-yl-acetamide

The procedure for Example 95 was followed utilizing 4-pyridyl acetic acid as the starting carboxylic acid. MS (ESI (+)) m/z 596.5 (M+H+).

### EXAMPLE 105

### N-{3-[4-(3-Morpholin-4-yl-3-oxo-propenyl)-2,3-bis-trifluoromethyl-phenylsulfanyl]-phenyl}-acetamide

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 400 μL of methylene chloride and acetic anhydride (7.37 μL, 0.078 mmol) was added. HPLC analysis showed the conversion of the starting material to the product quantitatively after stirring overnight at room temperature. The crude was diluted with DMSO and purified by preparative HPLC. MS (ESI (+)) m/z 518.7 (M+H+).

### EXAMPLE 106

### N-{3-[4-(3-Morpholin-4-yl-3-oxo-propenyl)-2,3-bis-trifluoromethyl-phenylsulfanyl]-phenyl}-2-piperazin-1-yl-acetamide

The procedure for Example 95 was followed utilizing 4-carboxymethyl-piperazine-1-carboxylic acid 9H-fluoren-9-ylmethyl ester as the starting carboxylic acid. The FMOC protected piperazine product was then deprotected with 2 mL of 2:8 piperidine:DMF. The reaction was concentrated after stirring at room temperature for 1 hr and diluted in DMSO for preparative HPLC purification. MS (ESI (+)) m/z 603.4 (M+H+).

### EXAMPLE 107

### N-{3-[4-(3-Morpholin-4-yl-3-oxo-propenyl)-2,3-bis-trifluoromethyl-phenylsulfanyl]-phenyl}-2-piperazin-1-yl-acetamide

The procedure for Example 95 was followed utilizing piperidine-1,2-dicarboxylic acid 1-tert-butyl ester as the starting carboxylic acid. The BOC protected piperidine product was then deprotected with 2 mL of 100% TFA. The reaction was concentrated after stirring at room temperature for 1 hr and diluted in DMSO for preparative HPLC purification. MS (ESI (+)) m/z 588.6 (M+H+).

### EXAMPLE 108

### Ethanesulfonic acid {2-[4-(3-morpholin-4-yl-3-oxo-propenyl)-2,3-bis-trifluoromethyl-phenylsulfanyl]-phenyl}-amide

A procedure similar to that utilized to obtain the product of Example 41 was used to obtain this compound. MS (ESI (+)) m/z 569.2 (M+H+). The starting aniline compound was prepared by using a procedure similar to that utilized to obtain the product of Example 4, except by using 2-aminothiophenol as the starting material.

### EXAMPLE 109

### 4-{2-[4-(3-Morpholin-4-yl-3-oxo-propenyl)-2,3-bis-trifluoromethyl-phenylsulfanyl]-phenylamino}-cyclohexanecarboxylic acid

A procedure similar to that utilized to obtain the product of Example 6 was used to obtain this compound. MS (ESI (+)) m/z 603.5 (M+H+). The starting aniline compound was prepared by using a procedure similar to that utilized to obtain the product of Example 4, except by using 2-aminothiophenol as the starting material.

### EXAMPLE 110

### N-{2-[4-(3-Morpholin-4-yl-3-oxo-propenyl)-2,3-bis-trifluoromethyl-phenylsulfanyl]-phenyl}-C-phenyl-methanesulfonamide

A procedure similar to that utilized to obtain the product of Example 41 was used to obtain this compound. MS (ESI (+)) m/z 631.4 (M+H+). The starting aniline compound was prepared by using a procedure similar to that utilized to obtain the product of Example 4, except by using 2-aminothiophenol as the starting material.

### EXAMPLE 111

### N-{2-[4-(3-Morpholin-4-yl-3-oxo-propenyl)-2,3-bis-trifluoromethyl-phenylsulfanyl]-phenyl}-benzenesulfonamide

A procedure similar to that utilized to obtain the product of Example 41 was used to obtain this compound. MS (ESI (+)) m/z 617.2 (M+H+). The starting aniline compound was prepared by using a procedure similar to that utilized to obtain the product of Example 4, except by using 2-aminothiophenol as the starting material.

### EXAMPLE 112

### 3-{2,3-Dichloro-4-[3-(tetrahydro-pyran-4-ylamino)-phenylsulfanyl]-phenyl}-1-morpholin-4-yl-propenone

A procedure similar to that utilized to obtain the product of Example 6 was used to obtain this compound from the corresponding dichloro aniline. MS (ESI (+)) m/z 492.9 (M+H+).

### EXAMPLE 113

### Cis 1-(3-{4-[3-(4-Carboxy-cyclohexylamino)-phenylsulfanyl]-2,3-bis-trifluoromethyl-phenyl}-acryloyl)-piperidine-3-carboxylic acid

A procedure similar to that utilized to obtain the product of Example 4 was used to obtain ethyl 3-[4-(3-amino-phenylsulfanyl)-2,3-bis-trifluoromethyl-phenyl]-propenoate, the starting ester. The starting ester (1.28 g, 2.84 mmol) was dissolved in 25.5 mL of THF and 4.50 mL of MeOH. A 2 N solution of lithium hydroxide (5.88 mL, 11.8 mmol) was added and the solution was stirred for 1 hour. After neutralizing with 24 mL of 1 N HCl, 100 mL of ethyl acetate (EtOAc) were added and the layers were separated. The organic layer washed with saturated NaCl solution, then dried over Na₂SO₄, filtered and concentrated in vacuo. The resulting solid was triturated with Et₂O/petroleum ether, then collected by filtration to afford an off-white solid (73%, 878 mg).

3-[4-(3-Amino-phenylsulfanyl)-2,3-bis-trifluoromethyl-phenyl]-propenoic acid (96.6 mg, 0.24 mmol) was added to a scintillation vial. A solution of 1-hydroxybenzotriazole (45.4 mg, 0.30 mmol) in 4.74 mL of DMF/CH₂Cl₂ was added to the vial. Ethyl nipecotate (46.1 μL, 0.30 mmol) and Et₃N (82.7 μL, 0.63 mmol) were added, followed by 1-(3-dimethylaminopropyl)-3-ethylcarbodiimide hydrocholoride (56.8 mg, 0.31 mmol). The reaction mixture was stirred for 3 days, then poured into 100 mL of 1 N HCl and extracted with 100 mL of EtOAc. The organic extracts were washed with 50 mL of saturated NaHCO₃ solution, 50 mL 1 N HCl, 50 mL of saturated NaHCO₃ solution, and 50 mL of saturated NaCl solution. The extracts were dried over Na₂SO₄, filtered and concentrated in vacuo to afford a foam. Purification by column chromatography using 3.5% MeOH/96.5% CH₂Cl₂ gave a white foam (93%, 121 mg).

1-{3-[4-(3-Amino-phenylsulfanyl)-2,3-bis-trifluoromethyl-phenyl]-acryloyl}-piperidine-3-carboxylic acid ethyl ester (110 mg, 0.20 mmol) was dissolved in 8.0 mL of 3% acetic acid (AcOH)/CH₂Cl₂. Ethyl 4-oxocyclohexanecarboxylate (95.4 μL, 0.60 mmol) was added and the reaction mixture was stirred for several minutes. Sodium triacetoxyborohydride (212 mg, 1.0 mmol) was added in one portion. After stirring overnight, the reaction mixture was diluted with 100 mL of EtOAc and washed with saturated NH₄Cl solution. The organic extract was dried over Na₂SO₄, filtered and concentrated in vacuo to afford an oil. Purification by column chromatography using 30% to 60% EtOAc/hexanes gave two products: cis isomer (51%, 72.4 mg), trans isomer (29%, 44.4 mg).

Cis 1-(3-{4-[3-(4-Ethoxycarbonyl-cyclohexylamino)-phenylsulfanyl]-2,3-bis-trifluoromethyl-phenyl}-acryloyl)-piperidine-3-carboxylic acid ethyl ester (72.4 mg, 0.10 mmol) was dissolved in 1.42 mL of 15% MeOH/THF. A solution of 2 N NaOH (200 μL, 0.40 mmol) was added and the reaction solution was rapidly stirred overnight. The reaction was quenched by addition of 400 μL of 1 N NaOH and stirred overnight. The solution was then evaporated under a stream of N₂ gas, and the resulting residue was redissolved in EtOAc. After washing with water, the organic extract was dried over Na₂SO₄, filtered and concentrated in vacuo. The resulting solid was triturated with hexanes/ether to afford the title compound as a white solid (97%, 62.5 mg). MS (ESI (+)) m/z 644.9 (M+H+).

### EXAMPLE 114

### Cis 4-(3-{4-[3-(3,6-Dihydro-2H-pyridin-1-yl)-3-oxo-propenyl]-2,3-bis-trifluoromethyl-phenylsulfanyl}-phenylamino)-cyclohexanecarboxylic acid

A procedure similar to that of Example 113 was used to obtain this compound wherein 3-[4-(3-amino-phenylsulfanyl)-2,3-bis-trifluoromethyl-phenyl]-propenoic acid was condensed with 1,2,3,6-tetrahydropyridine. MS (ESI (+)) m/z 598.9 (M+H+).

### EXAMPLE 115

### Cis 4-(3-{4-[2-(4-Methyl-piperazin-1-ylcarbamoyl)-vinyl]-2,3-bis-trifluoromethyl-phenylsulfanyl}-phenylamino)-cyclohexanecarboxylic acid

A procedure similar to that of Example 113 was used to obtain this compound wherein 3-[4-(3-amino-phenylsulfanyl)-2,3-bis-trifluoromethyl-phenyl]-propenoic acid was condensed with 1-amino-4-methyl-piperazine. MS (ESI (+)) m/z 631.1 (M+H+).

### EXAMPLE 116

### Cis 4-[3-(4-{2-[3-(2-Oxo-pyrrolidin-1-yl)-propylcarbamoyl]-vinyl}-2,3-bis-trifluoromethyl-phenylsulfanyl)-phenylamino]-cyclohexanecarboxylic acid

A procedure similar to that of Example 113 was used to obtain this compound wherein 3-[4-(3-amino-phenylsulfanyl)-2,3-bis-trifluoromethyl-phenyl]-propenoic acid was condensed with 1-(3-aminopropyl)-2-pyrrolidinone. MS (ESI (+)) m/z 658.2 (M+H+).

### EXAMPLE 117

### Cis 4-[3-(4-{3-[4-(2-Ethoxy-ethyl)-piperazin-1-yl]-3-oxo-propenyl}-2,3-bis-trifluoromethyl-phenylsulfanyl)-phenylamino]-cyclohexanecarboxylic acid

A procedure similar to that of Example 113 was used to obtain this compound wherein 3-[4-(3-amino-phenylsulfanyl)-2,3-bis-trifluoromethyl-phenyl]-propenoic acid was condensed with 1-(2-ethoxyethyl)piperazine. MS (ESI (+)) m/z 674.3 (M+H+).

### EXAMPLE 118

### Trans 4-[3-(4-{3-[4-(2-Ethoxy-ethyl)-piperazin-1-yl]-3-oxo-propenyl}-2,3-bis-trifluoromethyl-phenylsulfanyl)-phenylamino]-cyclohexanecarboxylic acid

A procedure similar to that of Example 113 was used to obtain this compound wherein 3-[4-(3-amino-phenylsulfanyl)-2,3-bis-trifluoromethyl-phenyl]-propenoic acid was condensed with. 1-(2-ethoxyethyl)piperazine and wherein the resulting trans isomer, trans-4-[3-(4-{3-[4-(2-ethoxy-ethyl)-piperazin-1-yl]-3-oxo-propenyl}-2,3-bis-trifluoromethyl-phenylsulfanyl)-phenylamino]-cyclohexanecarboxylic acid ethyl ester, was hydrolyzed with LiOH. MS (ESI (+)) m/z 674.3 (M+H+).

### EXAMPLE 119

### Cis 4-[3-(4-{3-[4-(2-Hydroxy-ethyl)-piperazin-1-yl]-3-oxo-propenyl}-2,3-bis-trifluoromethyl-phenylsulfanyl)-phenylamino]-cyclohexanecarboxylic acid

A procedure similar to that of Example 113 was used to obtain this compound wherein 3-[4-(3-amino-phenylsulfanyl)-2,3-bis-trifluoromethyl-phenyl]-propenoic acid was condensed with 1-(2-hydroxyethyl)piperazine. MS (ESI (+)) m/z 646.4 (M+H+).

### EXAMPLE 120

### Trans 4-[3-(4-{3-[4-(2-Hydroxy-ethyl)-piperazin-1-yl]-3-oxo-propenyl}-2,3-bis-trifluoromethyl-phenylsulfanyl)-phenylamino]-cyclohexanecarboxylic acid

A procedure similar to that of Example 113 was used to obtain this compound wherein 3-[4-(3-amino-phenylsulfanyl)-2,3-bis-trifluoromethyl-phenyl]-propenoic acid was condensed with 1-(2-hydroxyethyl)piperazine and wherein the resulting trans isomer, trans-4-[3-(4-{3-[4-(2-hydroxy-ethyl)-piperazin-1-yl]-3-oxo-propenyl}-2,3-bis-trifluoromethyl-phenylsulfanyl)-phenylamino]-cyclohexanecarboxylic acid ethyl ester, was hydrolyzed with LiOH. MS (ESI (+)) m/z 645.8 (M+H+).

### EXAMPLE 121

### 1-(3-{4-[3-(1-Methyl-piperidin-4-ylamino)-phenylsulfanyl]-2,3-bis-trifluoromethyl-phenyl}-acryloyl)-piperidine-4-carboxylic acid ethyl ester

A procedure similar to that utilized to obtain the product of Example 113 was used to obtain 3-[4-(3-amino-phenylsulfanyl)-2,3-bis-trifluoromethyl-phenyl]-propenoic acid, the starting acid. The starting acid (1.2 g, 2.7 mmol) and ethyl isonipacotate (1.3 g, 8.1 mmol) were dissolved in DMF and cooled to 0° C. Diisopropylethylamine (2.4 mL, 13.5 mmol) was added and the solution was stirred for 5 minutes. O-(7-Azobenzotriazol-1-yl)-N,N,N′,N′,-tetramethyluronium hexafluorophosphate (HATU) (1.4 g, 3.8 mmol) was added and the reaction mixture was allowed to warm to room temperature. The reaction mixture was diluted with 700 mL of EtOAc and washed twice with 75 mL of 10% HCl solution, twice with saturated NaHCO₃ solution, and four times with saturated NaCl solution. The extracts were dried over Mg₂SO₄, filtered and concentrated in vacuo to afford a viscous oil. Purification by column chromatography using 1.5% EtOH/98.5% EtOAc gave a pale yellow solid (85%, 1.43 g).

1-{3-[4-(3-Amino-phenylsulfanyl)-2,3-bis-trifluoromethyl-phenyl]-acryloyl}-piperidine-4-carboxylic acid ethyl ester (60 mg, 0.11 mmol), 1-methyl-4-piperidone (25 mg, 0.22 mmol) and AcOH (33 μL, 0.55 mmol) were dissolved in 1 mL of ClCH₂CH₂Cl at room temperature. Sodium triacetoxyborohydride (69 mg, 0.33 mmol) was added and a solution gradually formed. After stirring overnight, a 200 μL aliquot was quenched with several drops of TFA and purified by column chromatography to give 6.3 mg of the title compound. MS (ESI (+)) m/z 644.1 (M+H+).

### EXAMPLE 122

### 1-(3-{4-[3-(1-Methyl-piperidin-4-ylamino)-phenylsulfanyl]-2,3-bis-trifluoromethyl-phenyl}-acryloyl)-piperidine-4-carboxylic acid

A procedure similar to that utilized to obtain the product of Example 121 was used to obtain 1-(3-{4-[3-(1-methyl-piperidin-4-ylamino)-phenylsulfanyl]-2,3-bis-trifluoromethyl-phenyl}-acryloyl)-piperidine-4-carboxylic acid ethyl ester, the starting ester. To a solution of the starting ester in EtOH was added 8 equivalents of 2 N LiOH. After stirring at room temperature for 1 hour, another 4 equivalents of 2 N LiOH were added and the reaction mixture stirred for an additional 2 hours. Purification by column chromatography gave the product as a beige solid. MS (ESI (+)) m/z 615.9 (M+H+).

### EXAMPLE 123

### 1-(3-{4-[3-(Tetrahydro-pyran-4-ylamino)-phenylsulfanyl]-2,3-bis-trifluoromethyl-phenyl}-acryloyl)-piperidine-4-carboxylic acid

A procedure similar to that utilized to obtain the product of Example 121 was used to obtain 1-(3-{4-[3-(tetrahydro-pyran-4-ylamino)-phenylsulfanyl]-2,3-bis-trifluoromethyl-phenyl}-acryloyl)-piperidine-4-carboxylic acid ethyl ester. This ester was hydrolyzed according to the procedure of Example 122 to obtain the title compound. MS (ESI (+)) m/z 603.0 (M+H+).

### EXAMPLE 124

### 1-(3-{4-[3-(1,1-Dioxo-hexahydro-1λ⁶-thiopyran-4-ylamino)-phenylsulfanyl]-2,3-bis-trifluoromethyl-phenyl}-acryloyl)-piperidine-4-carboxylic acid

A procedure similar to that utilized to obtain the product of Example 121 was used to obtain 1-(3-{4-[3-(1,1-Dioxo-hexahydro-1λ⁶-thiopyran-4-ylamino)-phenylsulfanyl]-2,3-bis-trifluoromethyl-phenyl}-acryloyl)-piperidine-4-carboxylic acid ethyl ester. This ester was hydrolyzed according to the procedure of Example 122 to obtain the title compound. MS (ESI (+)) m/z 651.0 (M+H+).

### EXAMPLE 125

### [4-(3-{4-[3-(3-Methyl-ureido)-phenylsulfanyl]-2,3-bis-trifluoromethyl-phenyl}-acryloylamino)-phenyl]-acetic acid

A procedure similar to that utilized to obtain the product of Example 113 was used to obtain 3-[4-(3-amino-phenylsulfanyl)-2,3-bis-trifluoromethyl-phenyl]-propenoic acid, the starting acid. The starting acid (194 mg, 0.44 mmol) and diisopropylethylamine (391 μL, 2.2 mmol) were dissolved in CH₂Cl₂ at room temperature. Methyl isocyanate (75 μL, 1.3 mmol) was added in aliquots over 24 hours. The reaction mixture was then concentrated in vacuo and redissolved in EtOAc. The mixture washed twice with 10% HCl solution, once with water and once with saturated NaCl solution. The organic extract was dried over Na₂SO₄, filtered and concentrated in vacuo to afford a brown solid (99%, 221 mg).

3-{4-[3-(Methyl-ureido)-phenylsulfanyl]-2,3-bis-trifluoromethyl-phenyl}-propenoic acid (50 mg, 0.11 mmol), O-(7-Azobenzotriazol-1-yl)-N,N,N′,N′,-tetramethyluronium hexfluorophosphate (53 mg, 0.14 mmol), and diisopropylethylamine (77 μL, 0.44 mmol) were dissolved in DMF at room temperature. 4-Amino-phenylacetic acid ethyl ester (29 mg, 0.16 mmol) was immediately added and the reaction mixture was stirred for 1 hour. Methanol (500 μL) was then added, followed by 2 N LiOH (350 μL). Once the hydrolysis was complete by HPLC analysis, purification of the reaction mixture by column chromatography gave the title compound as a beige solid (33%, 21 mg). MS (ESI (+)) m/z 598.1 (M+H+).

### EXAMPLE 126

### N-(3-Hydroxy-propyl)-3-{4-[3-(3-methyl-ureido)-phenylsulfanyl]-2,3-bis-trifluoromethyl-phenyl}-acrylamide

A procedure similar to that utilized to obtain the product of Example 125 was used to obtain 3-{4-[3-(methyl-ureido)-phenylsulfanyl]-2,3-bis-trifluoromethyl-phenyl}-propenoic acid, the starting acid. The starting acid (41 mg, 0.088 mmol), O-(7-Azobenzotriazol-1-yl)-N,N,N′,N′,-tetramethyluronium hexfluorophosphate (44 mg, 0.11 mmol), and diisopropylethylamine (92 μL, 0.53 mmol) were dissolved in DMF. 3-Hydroxypropylamine (20 mg, 0.26 mmol) was added and the reaction was stirred until HPLC analysis indicated product formation was complete. Purification of the reaction mixture by column chromatography gave the title compound as a beige solid (50%, 23 mg). MS (ESI (+)) m/z 522.1 (M+H+).

### EXAMPLE 127

### N-(2-Hydroxy-1,1-dimethyl-ethyl)-3-{4-[3-(3-methyl-ureido)-phenylsulfanyl]-2,3-bis-trifluoromethyl-phenyl}-acrylamide

A procedure similar to that utilized to obtain the product of Example 126 was used to obtain this compound, wherein 2-hydroxy-1,1-dimethyl-ethylamine was used as the starting amine. MS (ESI (+)) m/z 536.1 (M+H+).

### EXAMPLE 128

### Thiophene-2-sulfonic acid [3-(4-{3-[4-(2-hydroxy-ethyl)-piperazin-1-yl]-3-oxo-propenyl}-2,3-bis-trifluoromethyl-phenylsulfanyl)-phenyl]-amide

A procedure similar to that utilized to obtain the product of Example 57 was used to obtain thiophene-2-sulfonic acid (3-{4-(3-ethoxycarbonyl-propenyl)-2,3-bis-trifluoromethyl-phenylsulfanyl}-phenyl])-amide. A procedure similar to that of Example 113 was used to hydrolyze the ethyl ester with 2 N LiOH to afford thiophene-2-sulfonic acid (3-{4-(3-carboxy-propenyl)-2,3-bis-trifluoromethyl-phenylsulfanyl}-phenyl])-amide. A procedure similar to that of Example 126 was used to couple the acid to 1-(2-hydroxyethyl)piperazine to obtain the title compound. MS (ESI (+)) m/z 665.9 (M+H+).

### EXAMPLE 129

### Trans 4-(3-{4-[2-(4-Carboxymethyl-phenylcarbamoyl)-vinyl]-2,3-bis-trifluoromethyl-phenylsulfanyl}-phenylamino)-cyclohexanecarboxylic acid

A procedure similar to that utilized to obtain the product of Example 113 was used to obtain trans 3-{4-[3-(4-ethoxycarbonyl-cyclohexylamino)-phenylsulfanyl]-2,3-bis-trifluoromethyl-phenyl}-propenoic acid. A procedure similar to Example 125 was used to couple the acid to 4-amino-phenylacetic acid ethyl ester to afford an amide and hydrolyze the ester functionalities of the resulting amide to obtain the title compound. MS (ESI (+)) m/z 667.2 (M+H+).

### EXAMPLE 130

### 1-[4-(2-Hydroxy-ethyl)-piperazin-1-yl]-3-{4-[3-(tetrahydro-pyran-4-ylamino)-phenylsulfanyl]-2,3-bis-trifluoromethyl-phenyl}-propenone

A procedure similar to that utilized to obtain the product of Example 121 was used to obtain the title compound, wherein 3-[4-(3-amino-phenylsulfanyl)-2,3-bis-trifluoromethyl-phenyl]-2-(hydroxy-ethyl)-piperazin-1-yl-propenone was obtained using 1-(2-hydroxyethyl)piperazine as the starting material. The amine was then condensed with tetrahydro-4H-pyran-4-one in a procedure similar to Example 113 to afford the title compound. MS (ESI (+)) m/z 604.6 (M+H+).

### EXAMPLE 131

### 1-[4-(2-Hydroxy-ethyl)-piperazin-1-yl]-3-{4-[3-(1-isopropyl-piperidin-4-ylamino)-phenylsulfanyl]-2,3-bis-trifluoromethyl-phenyl}-propenone

A procedure similar to that utilized to obtain the product of Example 130 was used to obtain this compound, wherein 3-[4-(3-amino-phenylsulfanyl)-2,3-bis-trifluoromethyl-phenyl]-2-(hydroxy-ethyl)-piperazin-1-yl-propenone was condensed with 1-isopropyl-4-piperidone. MS (ESI (+)) m/z 644.8 (M+H+).

### EXAMPLE 132

### (4-{3-[4-(3-Benzenesulfonylamino-phenylsulfanyl)-2,3-bis-trifluoromethyl-phenyl]-acryloylamino}-phenyl)-acetic acid

A procedure similar to that utilized to obtain the product of Example 41 was used to obtain 3-[4-(3-benzenesulfonylamino-phenylsulfanyl)-2,3-bis-trifluoromethyl-phenyl]-propenoic acid ethyl ester. A procedure similar to that of Example 113 was used to hydrolyze the ethyl ester with 2 N LiOH to afford 3-[4-(3-benzenesulfonylamino-phenylsulfanyl)-2,3-bis-trifluoromethyl-phenyl]-propenoic acid. A procedure similar to Example 125 was used to couple the acid to 4-amino-phenylacetic acid ethyl ester to afford an amide and hydrolyze the ester functionality of the resulting amide to obtain the title compound. MS (ESI (+)) m/z 681.1 (M+H+)

### EXAMPLE 133

### 3-{4-[3-(1-Ethyl-piperidin-4-ylamino)-phenylsulfanyl]-2,3-bis-trifluoromethyl-phenyl}-1-[4-(2-hydroxy-ethyl)-piperazin-1-yl]-propenone

A procedure similar to that utilized to obtain the product of Example 130 was used to obtain this compound, wherein 3-[4-(3-amino-phenylsulfanyl)-2,3-bis-trifluoromethyl-phenyl]-2-(hydroxy-ethyl)-piperazin-1-yl-propenone was condensed with 1-ethyl-4-piperidone. MS (ESI (+)) m/z 631.6 (M+H+).

### EXAMPLE 134

### 3-{2,3-Dichloro-4-[3-(1-ethyl-piperidin-4-ylamino)-phenylsulfanyl]-phenyl}-1-morpholin-4-yl-propenone

A procedure similar to that utilized to obtain the product of Example 19 was used to obtain this compound from the corresponding dichloro aniline. MS (ESI (+)) m/z 520.0 (M+H+).

### EXAMPLE 135

### 3-{2,3-Dichloro-4-[3-(1-propyl-piperidin-4-ylamino)-phenylsulfanyl]-phenyl}-1-morpholin-4-yl-propenone

A procedure similar to that utilized to obtain the product of Example 20 was used to obtain this compound from the corresponding dichloro aniline. MS (ESI (+)) m/z 534.3 (M+H+).

### EXAMPLE 136

### 3-{2,3-Dichloro-4-[3-(1-methyl-piperidin-4-ylamino)-phenylsulfanyl]-phenyl}-1-morpholin-4-yl-propenone

A procedure similar to that utilized to obtain the product of Example 18 was used to obtain this compound from the corresponding dichloro aniline. MS (ESI (+)) m/z 506.3 (M+H+).

### EXAMPLE 137

### 1-(3-{4-[3-(Phenylsulfonylamino)-phenylsulfanyl]-2,3-bis-trifluoromethyl-phenyl}-acryloyl)-piperidine-4-carboxylic acid ethyl ester

A procedure similar to that utilized to obtain the product of Example 121 is used to obtain 1-{3-[4-(3-amino-phenylsulfanyl)-2,3-bis-trifluoromethyl-phenyl]-acryloyl}-piperidine-4-carboxylic acid ethyl ester. A procedure similar to that utilized to obtain the product of Example 41 is used to obtain the title compound.

### EXAMPLE 138

### 1-{3-[4-(3-Aminophenylsulfanyl)-2,3-bis-trifluoromethyl-phenyl]-acryloylamido}-[2.2.2]-bicyclooctanyl-4-carboxylic acid methyl ester

A procedure similar to that utilized to obtain the product of Example 113 was used to obtain 3-[4-(3-amino-phenylsulfanyl)-2,3-bis-trifluoromethyl-phenyl]-propenoic acid. The acid was condensed with 4-amino-[2.2.2]-bicyclooctanyl-1-carboxylic acid methyl ester using a procedure similar to that of Example 121 to obtain the title compound. MS (ESI (+)) m/z 573.2 (M+H+).

### EXAMPLE 139

### 1-(3-{4-[3-(Phenylsulfonylamino)-phenylsulfanyl]-2,3-bis-trifluoromethyl-phenyl}-acryloylamido)-[2.2.2]-bicyclooctanyl-4-carboxylic acid

A procedure similar to that utilized to obtain the product of Example 138 was used to obtain 1-{3-[4-(3-aminophenylsulfanyl)-2,3-bis-trifluoromethyl-phenyl]-acryloylamido}-[2.2.2]-bicyclooctanyl-4-carboxylic acid methyl ester. The amine was acylated with phenylsulfonyl chloride using a procedure similar to that of Example 41 to obtain 1-(3-{4-[3-(phenylsulfonylamino)-phenylsulfanyl]-2,3-bis-trifluoromethyl-phenyl}-acryloylamido)-[2.2.2]-bicyclooctanyl-4-carboxylic acid methyl ester. The ester was hydrolyzed using a procedure similar to that of Example 113 to obtain the title compound. MS (ESI (+)) m/z 699.1 (M+H+).

### EXAMPLE 140

### 1-(3-{4-[3-(1-Methylpiperidin-4-ylamino)-phenylsulfanyl]-2,3-bis-trifluoromethyl-phenyl}-acryloylamido)-[2.2.2]-bicyclooctanyl-4-carboxylic acid

A procedure similar to that utilized to obtain the product of Example 138 was used to obtain 1-{3-[4-(3-aminophenylsulfanyl)-2,3-bis-trifluoromethyl-phenyl]-acryloylamido}-[2.2.2]-bicyclooctanyl-4-carboxylic acid methyl ester. A procedure similar to that of Example 113 was used to couple the amine to 1-methyl-4-piperidone and hydrolyze the methyl ester with LiOH to obtain the title compound. MS (ESI (+)) m/z 656.2 (M+H+).

### EXAMPLE 141

### 1-(3-{4-[3-(1-Morpholin-4-yl)-phenylsulfanyl]-2,3-bis-trifluoromethyl-phenyl}-acryloylamido)-[2.2.2]-bicyclooctanyl-4-carboxylic acid

A procedure similar to that utilized to obtain the product of Example 138 was used to obtain 1-{3-[4-(3-aminophenylsulfanyl)-2,3-bis-trifluoromethyl-phenyl]-acryloylamido}-[2.2.2]-bicyclooctanyl-4-carboxylic acid methyl ester. A procedure similar to that of Example 113 was used to couple the amine to tetrahydro-4H-pyran-4-one and hydrolyze the methyl ester with LiOH to obtain the title compound. MS (ESI (+)) m/z 643.2 (M+H+).

### EXAMPLE 142

### 1-(3-{4-[3-(1,1-Dioxo-hexahydro-1λ⁶-thiopyran-4-ylamino)-phenylsulfanyl]-2,3-bis-trifluoromethyl-phenyl}-acryloylamido)-[2.2.2]-bicyclooctanyl-4-carboxylic acid

A procedure similar to that utilized to obtain the product of Example 138 was used to obtain 1-{3-[4-(3-aminophenylsulfanyl)-2,3-bis-trifluoromethyl-phenyl]-acryloylamido}-[2.2.2]-bicyclooctanyl-4-carboxylic acid methyl ester. A procedure similar to that of Example 113 was used to couple the amine to 1,1-dioxo-hexahydro-1λ⁶-thiopyran-4-one and hydrolyze the methyl ester with LiOH to obtain the title compound. MS (ESI (+)) m/z 691.6 (M+H+).

### EXAMPLE 143

### 3-[4-(2-Hydroxy-phenylsulfanyl)-2,3-bis-trifluoromethyl-phenyl]-1-morpholin-4-yl-propenone

Trifluoromethanesulfonic acid 4-(3-morpholin-4-yl-3-oxo-propenyl)-2,3-bis-trifluoromethyl-phenyl ester (0.96 g, 1.9 mmol, Example 3) was azeotroped twice with toluene, and then dissolved in 5 mL of acetone. Potassium carbonate (0.37 g, 2.7 mmol) was dried by heating under vacuum, and then added to an acetone solution of 2-hydroxythiophenol (0.35 g, 2.8 mmol in 5 mL of acetone). To this mixture was added the triflate solution, followed by heating at reflux overnight. The reaction was concentrated, then partitioned between ethyl acetate and 1 N aqueous hydrochloric acid. The organic layer washed with saturated aqueous sodium chloride, dried with sodium sulfate, filtered and concentrated. The residue was purified by column chromatography 1:3-3:1 ethyl acetate/hexanes (18%, 161 mg). ¹H NMR (CDCl₃, 300 MHz) δ 3.55-3.71 (m, 8H), 6.53 (d, J=15.4 Hz, 1H), 6.99 (d, J=8.5 Hz, 1H), 7.02 (td, J=7.8, 1.2 Hz), 7.11 (dd, J=1.3, 8.4 Hz, 1H), 7.40 (d, J=8.5 Hz, 1H), 7.47 (ddd, J=1.8, 7.5, 8.4 Hz, 1H), 7.52 (dd, J=1.8, 7.5 Hz, 1H), 7.83 (dq, J=14.3, 4.2 Hz, 1H); MS (ESI (+)) m/z 478.0 (M+H⁺).

### EXAMPLE 144

### 3-[4-(3-Hydroxy-phenylsulfanyl)-2,3-bis-trifluoromethyl-phenyl]-1-morpholin-4-yl-propenone

The procedure of Example 143 was followed utilizing 3-hydroxythiophenol as the starting thiophenol. MS (ESI (+)) m/z 478.0 (M+H⁺).

### EXAMPLE 145

### 1-Morpholin-4-yl-3-{4-[2-(tetrahydro-thiopyran-4-yloxy)-phenylsulfanyl]-2,3-bis-trifluoromethyl-phenyl}-propenone

3-[4-(2-Hydroxy-phenylsulfanyl)-2,3-bis-trifluoromethyl-phenyl]-1-morpholin-4-yl-propenone (30 mg, 0.063 mmol, Example 143), tetrahydro-thiopyran-4-ol (30 mg, 0.25 mmol), and triphenylphosphine (68 mg, 0.26 mmol) were dissolved in THF (1 mL). Diisopropylazodicarboxylate (0.050 mL, 0.25 mmol) was added, and the solution agitated overnight. The reaction was evaporated to dryness, and purified by preparative HPLC to give the product (24%, 8.8 mg). ¹H NMR (DMSO-d₆, 400 MHz) δ 1.54 (m, 2H), 1.85 (m, 2H), 2.29-2.47 (m, 4H), 3.55-3.68 (m, 8H), 4.52 (m, 1H), 7.05 (t, J=7.6 Hz, 1H), 7.14 (d, J=15 Hz, 1H), 7.15 (d, J=7.6 Hz, 1H), 7.18 (d, J=8.7 Hz, 1H), 7.47 (td, J=7.8, 1.8 Hz), 7.61 (dd, J=1.6, 7.7 Hz, 1H), 7.66 (dq, J=15.3, 4.1 Hz, 1H), 7.95 (d, J=8.8 Hz, 1H); MS (ESI (+)) m/z 578.3 (M+H⁺).

### EXAMPLE 146

### 1-Morpholin-4-yl-3-{4-[3-(tetrahydro-thiopyran-4-yloxy)-phenylsulfanyl]-2,3-bis-trifluoromethyl-phenyl}-propenone

The procedure for Example 145 was followed utilizing Example 144 as the starting phenol. MS (ESI (+)) m/z 578.4 (M+H⁺).

### EXAMPLE 147

### 1-Morpholin-4-yl-3-{4-[2-(pyridin-2-ylmethoxy)-phenylsulfanyl]-2,3-bis-trifluoromethyl-phenyl}-propenone

The procedure for Example 145 was followed utilizing pyridin-2-yl-methanol as the starting alcohol. MS (ESI (+)) m/z 569.0 (M+H⁺)

### EXAMPLE 148

### 1-Morpholin-4-yl-3-{4-[2-(Pyridin-3-ylmethoxy)-phenylsulfanyl]-2,3-bis-trifluoromethyl-phenyl}-propenone

The procedure for Example 145 was followed utilizing pyridin-3-yl-methanol as the starting alcohol. MS (ESI (+)) m/z 569.0 (M+H⁺).

### EXAMPLE 149

### 1-Morpholin-4-yl-3-{4-[2-(Pyridin-4-ylmethoxy)-phenylsulfanyl]-2,3-bis-trifluoromethyl-phenyl}-propenone

The procedure for Example 145 was followed utilizing pyridin-4-yl-methanol as the starting alcohol. MS (ESI (+)) m/z 569.1 (M+H⁺).

### EXAMPLE 150

### 1-Morpholin-4-yl-3-{4-[2-(2-pyridin-2-yl-ethoxy)-phenylsulfanyl]-2,3-bis-trifluoromethyl-phenyl}-propenone

The procedure for Example 145 was followed utilizing 2-pyridin-2-yl-ethanol as the starting alcohol. MS (ESI (+)) m/z 583.1 (M+H⁺).

### EXAMPLE 151

### 3-[4-(2-Benzyloxy-phenylsulfanyl)-2,3-bis-trifluoromethyl-phenyl]-1-morpholin-4-yl-propenone

The procedure for Example 145 was followed utilizing benzyl alcohol as the starting alcohol. MS (ESI (+)) m/z 568.1 (M+H⁺).

### EXAMPLE 152

### 3-[4-(2-Cyclohexyloxy-phenylsulfanyl)-2,3-bis-trifluoromethyl-phenyl]-1-morpholin-4-yl-propenone

The procedure for Example 145 was followed utilizing cyclohexanol as the starting alcohol. MS (ESI (+)) m/z 560.2 (M+H⁺).

### EXAMPLE 153

### 3-[4-(3-Cyclohexyloxy-phenylsulfanyl)-2,3-bis-trifluoromethyl-phenyl]-1-morpholin-4-yl-propenone

The procedure for Example 145 was followed utilizing cyclohexanol as the starting alcohol and 3-[4-(3-hydroxy-phenylsulfanyl)-2,3-bis-trifluoromethyl-phenyl]-1-morpholin-4-yl-propenone (Example 144) as the starting phenol. MS (ESI (+)) m/z 560.3 (M+H+).

### EXAMPLE 154

### 3-{4-[2-(trans-4-Methyl-cyclohexyloxy)-phenylsulfanyl]-2,3-bis-trifluoromethyl-phenyl}-1-morpholin-4-yl-propenone

The procedure for Example 145 was followed utilizing cis-4-methylcyclohexanol as the starting alcohol. MS (ESI (+)) m/z 574.2 (M+H⁺).

### EXAMPLE 155

### 3-{4-[3-(trans-4-Methyl-cyclohexyloxy)-phenylsulfanyl]-2,3-bis-trifluoromethyl-phenyl}-1-morpholin-4-yl-propenone

The procedure for Example 145 was followed utilizing cis-4-methylcyclohexanol as the starting alcohol and 3-[4-(3-hydroxy-phenylsulfanyl)-2,3-bis-trifluoromethyl-phenyl]-1-morpholin-4-yl-propenone (Example 144) as the starting phenol. MS (ESI (+)) m/z 574.3 (M+H⁺).

### EXAMPLE 156

### 3-{4-[2-(cis-4-Methyl-cyclohexyloxy)-phenylsulfanyl]-2,3-bis-trifluoromethyl-phenyl}-1-morpholin-4-yl-propenone

The procedure for Example 145 was followed utilizing trans-4-methylcyclohexanol as the starting alcohol. MS (ESI (+)) m/z 574.3 (M+H⁺).

### EXAMPLE 157

### 3-{4-[3-(cis-4-Methyl-cyclohexyloxy)-phenylsulfanyl]-2,3-bis-trifluoromethyl-phenyl}-1-morpholin-4-yl-propenone

The procedure for Example 145 was followed utilizing trans-4-methylcyclohexanol as the starting alcohol and 3-[4-(3-hydroxy-phenylsulfanyl)-2,3-bis-trifluoromethyl-phenyl]-1-morpholin-4-yl-propenone (Example 144) as the starting phenol. MS (ESI (+)) m/z 574.4 (M+H⁺).

### EXAMPLE 158

### 1-Morpholin-4-yl-3-{4-[2-(tetrahydro-pyran-4-yloxy)-phenylsulfanyl]-2,3-bis-trifluoromethyl-phenyl}-propenone

The procedure for Example 145 was followed utilizing tetrahydro-pyran-4-ol as the starting alcohol. MS (ESI (+)) m/z 562.2 (M+H⁺).

### EXAMPLE 159

### 1-Morpholin-4-yl-3-{4-[3-(tetrahydro-pyran-4-yloxy)-phenylsulfanyl]-2,3-bis-trifluoromethyl-phenyl}-propenone

The procedure for Example 145 was followed utilizing tetrahydro-pyran-4-ol as the starting alcohol and 3-[4-(3-hydroxy-phenylsulfanyl)-2,3-bis-trifluoromethyl-phenyl]-1-morpholin-4-yl-propenone (Example 144) as the starting phenol. MS (ESI (+)) m/z 562.3 (M+H⁺).

### EXAMPLE 160

### 1-Morpholin-4-yl-3-{4-[2-(thiophen-2-ylmethoxy)-phenylsulfanyl]-2,3-bis-trifluoromethyl-phenyl}-propenone

Resin-bound triphenylphosphine (164 mg, 1.1 mmol/g, 0.18 mmol) was swelled with methylene chloride, then washed three times with methylene chloride. After drying, the beads were swelled in methylene chloride (4 mL). 3-[4-(2-hydroxy-phenylsulfanyl)-2,3-bis-trifluoromethyl-phenyl]-1-morpholin-4-yl-propenone (19 mg, 0.040 mmol, Example 143) was added and the mixture was shaken for 5 minutes. Thiophen-2-yl-methanol (0.020 mL, 0.21 mmol) was added and the mixture shaken for 5 minutes. Diisopropylazodicarboxylate (0.033 mL, 0.17 mmol) was added and the reaction shaken for 1 h. The resin was filtered off and washed with methylene chloride. The organic layers were combined and concentrated to dryness. Purification by preparative HPLC gave the product (24%, 5.5 mg). ¹H NMR (DMSO-d₆, 300 MHz) δ 3.51-3.69 (m, 8H), 5.23 (s, 2H), 6.88-7.17 (m, 5H), 7.31 (dd, J=0.9, 8.6 Hz, 1H), 7.40 (dd, J=1.3, 5.1 Hz, 1H), 7.41-7.56 (m, 1H), 7.57 (dd, J=1.7, 7.5 Hz, 1H), 7.65 (dq, J=15.3, 4.1 Hz, 1H), 7.90 (d, J=8.7 Hz, 1H); MS (ESI (+)) m/z 574.2 (M+H⁺).

### EXAMPLE 161

### 1-Morpholin-4-yl-3-{4-[2-(2-thiophen-3-yl-ethoxy)-phenylsulfanyl]-2,3-bis-trifluoromethyl-phenyl}-propenone

The procedure for Example 160 was followed utilizing 2-thiophen-3-yl-ethanol as the starting alcohol. MS (ESI (+)) m/z 588.2 (M+H⁺).

### EXAMPLE 162

### 3-[4-(3-Benzyloxy-phenylsulfanyl)-2,3-bis-trifluoromethyl-phenyl]-1-morpholin-4-yl-propenone

The procedure for Example 160 was followed utilizing benzyl alcohol as the starting alcohol and 3-[4-(3-hydroxy-phenylsulfanyl)-2,3-bis-trifluoromethyl-phenyl]-1-morpholin-4-yl-propenone (Example 144) as the starting phenol. MS (ESI (+)) m/z 568.1 (M+H⁺).

### EXAMPLE 163

### 3-{4-[3-(1H-Imidazol-4-ylmethoxy)-phenylsulfanyl]-2,3-bis-trifluoromethyl-phenyl}-1-morpholin-4-yl-propenone

3-[4-(3-Hydroxy-phenylsulfanyl)-2,3-bis-trifluoromethyl-phenyl]-1-morpholin-4-yl-propenone (43 mg, 0.090 mmol, Example 144) was dissolved in ethanol (1.25 mL). To this was added a solution of sodium ethoxide in ethanol (0.084 mL, 21%, 0.23 mmol). After stirring at room temperature for 30 minutes, 4-chloromethyl-1H-imidazole hydrochloride salt (24 mg, 0.16 mmol) was added, and the reaction stirred for 30 minutes. Analysis by HPLC showed >75% conversion. Trifluoroacetic acid (0.035 mL) was added, and the reaction was evaporated to dryness. Purification by preparative HPLC gave the product. ¹H NMR (DMSO-d₆, 400 MHz) δ 3.35-3.74 (m, 8H), 5.19 (s, 2H), 7.12 (d, J=7.5 Hz, 1H), 7.14-7.22 (m, 3H), 7.35 (d, J=8.4 Hz, 1H), 7.44 (t, J=7.8 Hz, 1H), 7.67 (dq, J=15.0, 4.5 Hz, 1H), 7.78 (s, 1H), 8.02 (d, J=8.8 Hz, 1H), 9.09 (s, 1H); MS (ESI (+)) m/z 558.0 (M+H⁺).

### EXAMPLE 164

### 3-{4-[2-(1H-Imidazol-4-ylmethoxy)-phenylsulfanyl]-2,3-bis-trifluoromethyl-phenyl}-1-morpholin-4-yl-propenone

The procedure for Example 163 was followed utilizing 3-[4-(2-hydroxy-phenylsulfanyl)-2,3-bis-trifluoromethyl-phenyl]-1-morpholin-4-yl-propenone (Example 143) as the starting phenol. MS (ESI (+)) m/z 558.4 (M+H⁺).

### EXAMPLE 165

### Tans-4-{2-[4-(3-Morpholin-4-yl-3-oxo-propenyl)-2,3-bis-trifluoromethyl-phenylsulfanyl]-phenoxy}-cyclohexanecarboxylic acid

Hydroxy-phenylsulfanyl-2,3-bis-trifluoromethyl-phenyl]-1-morpholin-4-yl-propenone (51 mg, 0.11 mmol, Example 143), cis-4-hydroxy-cyclohexanecarboxylic acid methyl ester (68 mg, 0.43 mmol), triphenylphosphine (117 mg, 0.45 mmol) were dissolved in THF (1.25 mL). Diisopropylazodicarboxylate (0.084 mL, 0.43 mmol) was added, and the solution stirred overnight at 80° C. in a sealed tube. The reaction was evaporated to dryness, and purified by preparative HPLC to give the ether. This material (48 mg, 0.078 mmol) was dissolved in THF (1.5 mL) and MeOH (1.5 mL). LiOH (1.5 mL, 2 N) was added and the reaction stirred for three hours. The reaction was evaporated to dryness, then partitioned between ethyl acetate and 1 N hydrochloric acid. The organic layer washed with saturated sodium chloride, dried with sodium sulfate, filtered and evaporated. The residue was purified by preparative HPLC to give the product (36%, 24 mg). ¹H NMR (DMSO-d₆, 300 MHz) δ 1.00 (m, 2H), 1.41 (m, 2H), 1.72 (m, 4H), 2.03 (m, 1H), 3.50-3.70 (m, 8H), 4.30 (m, 1H), 7.02 (t, J=7.7 Hz, 1H), 7.15 (d, J=15.0 Hz, 1H), 7.16 (d, J=8.3 Hz, 1H), 7.22 (d, J=8.3 Hz, 1H), 7.45 (td, J=8.0, 1.8 Hz, 1H), 7.58 (dd, J=1.7, 8.0 Hz, 1H), 7.66 (dq, J=15.1, 4.4 Hz, 1H), 7.95 (d, J=8.4 Hz, 1H).

### EXAMPLE 166

### Cis-4-{2-[4-(3-Morpholin-4-yl-3-oxo-propenyl)-2,3-bis-trifluoromethyl-phenylsulfanyl]-phenoxymethyl}-cyclohexanecarboxylic acid

The procedure for Example 165 was followed utilizing trans-4-hydroxymethyl-cyclohexanecarboxylic acid methyl ester as the starting alcohol. MS (ESI (+)) m/z 618.2 (M+H⁺).

### EXAMPLE 167

### Trans-4-{2-[4-(3-Morpholin-4-yl-3-oxo-propenyl)-2,3-bis-trifluoromethyl-phenylsulfanyl]-phenoxymethyl}-cyclohexanecarboxylic acid

The procedure for Example 165 was followed utilizing trans-4-hydroxymethyl-cyclohexanecarboxylic acid methyl ester as the starting alcohol. MS (ESI (+)) m/z 618.4 (M+H⁺).

### EXAMPLE 168

### Cis-4-{3-[4-(3-Morpholin-4-yl-3-oxo-propenyl)-2,3-bis-trifluoromethyl-phenylsulfanyl]-phenoxymethyl}-cyclohexanecarboxylic acid

The procedure for Example 165 was followed utilizing cis-4-hydroxymethyl-cyclohexanecarboxylic acid methyl ester as the starting alcohol. MS (ESI (+)) m/z 618.3 (M+H⁺).

### EXAMPLE 169

### Trans-4-{3-[4-(3-Morpholin-4-yl-3-oxo-propenyl)-2,3-bis-trifluoromethyl-phenylsulfanyl]-phenoxymethyl}-cyclohexanecarboxylic acid

The procedure for Example 165 was followed utilizing trans-4-hydroxymethyl-cyclohexanecarboxylic acid methyl ester as the starting alcohol and hydroxy-phenylsulfanyl)-2,3-bis-trifluoromethyl-phenyl]-1-morpholin-4-yl-propenone (Example 144) as the starting phenol. MS (ESI (+)) m/z 561.3 (M+H⁺).

### EXAMPLE 170

### 1-Morpholin-4-yl-3-{4-[2-(piperidin-4-yloxy)-phenylsulfanyl]-2,3-bis-trifluoromethyl-phenyl}-propenone

3-[4-(2-hydroxy-phenylsulfanyl)-2,3-bis-trifluoromethyl-phenyl]-1-morpholin-4-yl-propenone (30 mg, 0.063 mmol, Example 143), 4-hydroxy-piperidine-1-carboxylic acid tert-butyl ester (52 mg, 0.26 mmol), triphenylphosphine (68 mg, 0.26 mmol) were dissolved in THF (1 mL). Diisopropylazodicarboxylate (0.050 mL, 0.25 mmol) was added, and the solution agitated overnight. The reaction was evaporated to dryness, and purified by preparative HPLC to give the ether. This material was dissolved in methylene chloride (1 mL). Trifluoroacetic acid (1 mL) was added and the reaction stirred for 1 h. The reaction was evaporated to dryness, and the residue was purified by preparative HPLC to give the product (35%, 14.9 mg). ¹H NMR (DMSO-d₆, 400 MHz) δ 1.58 (m, 2H), 1.89 (m, 2H), 3.01 (m, 4H), 3.35-3.80 (m, 8H), 4.67 (m, 1H), 7.09 (t, J=7.9 Hz, 1H), 7.16 (d, J=115.1 Hz, 1H), 7.19 (d, J=8.2 Hz, 1H), 7.25 (d, J=8.5 Hz, 1H), 7.51 (td, J=7.8, 1.5 Hz, 1H), 7.55 (dd, J=1.4, 7.6 Hz, 1H), 7.67 (dq, J=15.1, 4.1 Hz, 1H), 7.96 (d, J=8.6 Hz, 1H), 8.41 (br s, 1H); MS (ESI (+)) m/z 561.3 (M+H⁺).

### EXAMPLE 171

### 1-Morpholin-4-yl-3-{4-[3-(piperidin-4-yloxy)-phenylsulfanyl]-2,3-bis-trifluoromethyl-phenyl}-propenone

The procedure for Example 170 was followed utilizing 3-[4-(3-hydroxy-phenylsulfanyl)-2,3-bis-trifluoromethyl-phenyl]-1-morpholin-4-yl-propenone (Example 144) as the starting phenol. MS (ESI (+)) m/z 561.3 (M+H⁺).

### EXAMPLE 172

### 4-{3-[4-(3-Morpholin-4-yl-3-oxo-propenyl)-2,3-bis-trifluoromethyl-phenylsulfanyl]-phenylamino}-piperidine-1-carboxylic acid ethyl ester

The product of Example 4 was subjected to the procedure described in Example 8 utilizing N-(t-butoxycarbonyl)-piperazine as the starting material, followed by hydrolysis described in Example 191. The crude product was dissolved in DCM, treated with an excess of diisopropylethyl amine and ethyl chloroformate to afford the final product, purified by HPLC. MS (ESI (+)) m/z 614 (M+H⁺).

### EXAMPLE 173

### 3-(4-{3-[1-(2,2-Dimethyl-propionyl)-piperidin-4-ylamino]-phenylsulfanyl}-2,3-bis-trifluoromethyl-phenyl)-1-morpholin-4-yl-propenone

The procedure for Example 172 was followed utilizing 2,2-dimethylpropionyl chloride as the starting acyl chloride. MS (ESI (+)) m/z 626 (M+H⁺).

### EXAMPLE 174

### 3-(4-{3-[1-(2-Methoxy-acetyl)-piperidin-4-ylamino]-phenylsulfanyl}-2,3-bis-trifluoromethyl-phenyl)-1-morpholin-4-yl-propenone

The procedure for Example 172 was followed utilizing methoxyacetyl chloride as the starting acyl chloride. MS (ESI (+)) m/z 614 (M+H⁺).

### EXAMPLE 175

### 3-Methyl-1-(4-{3-[4-(3-morpholin-4-yl-3-oxo-propenyl)-2,3-bis-trifluoromethyl-phenylsulfanyl]-phenylamino}-piperidin-1-yl)-butan-1-one

The procedure for Example 172 was followed utilizing 3-methyl-butyryl chloride as the starting acyl chloride. MS (ESI (+)) m/z 627 (M+H⁺).

### EXAMPLE 176

### 3-[4-(3-{1-[2-(2-Methoxy-ethoxy)-acetyl]-piperidin-4-ylamino}-3-phenylsulfanyl)-2,3-bis-trifluoromethyl-phenyl]-1-morpholin-4-yl-propenone

The procedure for Example 172 was followed utilizing (2-methoxy-ethoxy)-acetyl chloride as the starting acyl chloride. MS (ESI (+)) m/z 658 (M+H⁺).

### EXAMPLE 177

### 3-{4-[3-(1-Isobutyryl-piperidin-4-ylamino)-phenylsulfanyl]-2,3-bis-trifluoromethyl-phenyl}-1-morpholin-4-yl-propenone

The procedure for Example 172 was followed utilizing isobutyryl chloride as the starting acyl chloride. MS (ESI (+)) m/z 612 (M+H⁺).

### EXAMPLE 178

### 4-{3-[4-(3-Morpholin-4-yl-3-oxo-propenyl)-2,3-bis-trifluoromethyl-phenylsulfanyl]-phenylamino}-piperidine-1-carboxylic acid isopropyl ester

The procedure for Example 172 was followed utilizing isopropyl chloroformate as the starting acyl chloride. MS (ESI (+)) m/z 628 (M+H⁺).

### EXAMPLE 179

### 3-(4-{3-[1-(2-Dimethylamino-acetyl)-piperidin-4-ylamino]-phenylsulfanyl}-2,3-bis-trifluoromethyl-phenyl)-1-morpholin-4-yl-propenone

The procedure for Example 172 was followed utilizing dimethylamino-acetyl chloride as the starting acyl chloride. MS (ESI (+)) m/z 627 (M+H⁺).

### EXAMPLE 180

### 4-{3-[4-(3-Morpholin-4-yl-3-oxo-propenyl)-2,3-bis-trifluoromethyl-phenylsulfanyl]-phenylamino}-piperidine-1-carboxylic acid 2-methoxy-ethyl ester

The procedure for Example 172 was followed utilizing methoxyethyl chloroformate as the starting acyl chloride. MS (ESI (+)) m/z 644 (M+H⁺).

### EXAMPLE 181

### 3-{4-[3-(1-Cyclopropyl-piperidin-4-ylamino)-phenylsulfanyl]-2,3-bis-trifluoromethyl-phenyl}-1-morpholin-4-yl-propenone

The procedure for Example 172 was followed utilizing (1-ethoxy-cyclopropoxy)-trimethylsilane as the alkylating reagent. MS (ESI (+)) m/z 582 (M+H⁺).

### EXAMPLE 182

### 3-(4-{3-[1-(3-Methoxy-propionyl)-piperidin-4-ylamino]-phenylsulfanyl}-2,3-bis-trifluoromethyl-phenyl)-1-morpholin-4-yl-propenone

The procedure for Example 172 was followed utilizing 3-methoxy-propionyl chloride as the starting acyl chloride. MS (ESI (+)) m/z 628 (M+H⁺).

### EXAMPLE 183

### 4-{3-[4-(3-Morpholin-4-yl-3-oxo-propenyl)-2,3-bis-trifluoromethyl-phenylsulfanyl]-phenylamino}-piperidine-1-carboxylic acid allyl ester

The procedure for Example 172 was followed utilizing 2-propenyl chloroformate as the starting acyl chloride. MS (ESI (+)) m/z 626 (M+H⁺).

### EXAMPLE 184

### 2-Methyl-4-{3-[4-(3-morpholin-4-yl-3-oxo-propenyl)-2,3-bis-trifluoromethyl-phenylsulfanyl]-phenylamino}-piperidine-1-carboxylic acid tert-butyl ester

The procedure for Example 8 was followed utilizing 2-methyl-4-oxo-piperidine-1-carboxylic acid tert-butyl ester as the starting ketone. MS (ESI (+)) m/z 656 (M+H⁺).

### EXAMPLE 185

### 1-(4-Methyl-piperazin-1-yl)-3-{4-[3-(tetrahydro-pyran-4-ylamino)-phenylsulfanyl]-2,3-bis-trifluoromethyl-phenyl}-propenone

3-Morpholin-4-yl-1-{4-[3-(tetrahydro-pyran-4-ylamino)-phenylsulfanyl]-2,3-bis-trifluoromethyl-phenyl}-propenone was hydrolyzed with KOH (3 eq.) in MeOH over period of 24 hrs., and concentrated. The resulting acid and diisopropylethyl amine were dissolved in DMF. HATU was added, and after stirring for a few minutes at room temperature, 1-methyl-piperazine was added. The reaction was stirred overnight to give the desired product. MS (ESI (+)) m/z 556 (M+H⁺).

### EXAMPLE 186

### 1-[4-(2-Hydroxy-ethyl)-piperidin-1-yl]-3-{4-[3-(tetrahydro-pyran-4-ylamino)-phenylsulfanyl]-2,3-bis-trifluoromethyl-phenyl}-propenone

The procedure for Example 185 was followed utilizing 4-(2-hydroxyethyl)-piperidine as the starting amine. MS (ESI (+)) m/z 585 (M+H⁺).

### EXAMPLE 187

### 3-(4-{3-[1-(2-Hydroxy-ethyl)-piperidin-4-ylamino]-phenylsulfanyl}-2,3-bis-trifluoromethyl-phenyl)-1-morpholin-4-yl-propenone

The procedure for Example 172 was followed utilizing 2-bromo-ethanol as the alkylating reagent. MS (ESI (+)) m/z 586 (M+H⁺).

### EXAMPLE 188

### 3-(4-{3-[1-(2-Methoxy-ethyl)-piperidin-4-ylamino]-phenylsulfanyl}-2,3-bis-trifluoromethyl-phenyl)-1-morpholin-4-yl-propenone

The procedure for Example 172 was followed utilizing 1-chloro-2-methoxy-ethane as the alkylating reagent. MS (ESI (+)) m/z 600 (M+H⁺).

### EXAMPLE 189

### 3-(4-{3-[1-(1-Methylamino-cyclopropanecarbonyl)-piperidin-4-ylamino]-phenylsulfanyl}-2,3-bis-trifluoromethyl-phenyl)-1-morpholin-4-yl-propenone

The procedure for Example 172 was followed utilizing 1-methylamino-cyclopropane-1-carbonyl chloride as the acyl chloride. MS (ESI (+)) m/z 639 (M+H⁺).

### EXAMPLE 190

### 4-(3-{4-[3-(Tetrahydro-pyran-4-ylamino)-phenylsulfanyl]-2,3-bis-trifluoromethyl-phenyl}-acryloyl)-piperazine-1-carboxylic acid tert-butyl ester

The procedure for Example 185 was followed 1-(t-butoxycarbonyl)-piperazine as the starting amine. MS (ESI (+)) m/z 642 (M+H⁺).

### EXAMPLE 191

### 1-piperazin-1-yl-3-{4-[3-(tetrahydro-pyran-4-ylamino)-phenylsulfanyl]-2,3-bis-trifluoromethyl-phenyl}-propenone

Example 190 was hydrolyzed with TFA in DCM over a period of 1 hr. MS (ESI (+)) m/z 542 (M+H⁺).

### EXAMPLE 192

### 2-Methylamino-N-{3-[4-(3-morpholin-4-yl-3-oxo-propenyl)-2,3-bis-trifluoromethyl-phenylsulfanyl]-phenyl}-acetamide

The product from Example 4 was dissolved in DCM and treated with an excess of diisopropylethyl amine and bromoacetyl chloride. The product from this reaction was further treated with methyl amine to afford the desired product. MS (ESI (+)) m/z 530 (M+H⁺).

### EXAMPLE 193

### 3-Methylamino-N-{3-[4-(3-morpholin-4-yl-3-oxo-propenyl)-2,3-bis-trifluoromethyl-phenylsulfanyl]-phenyl}-propionamide

The procedure for Example 192 was followed utilizing 3-bromopropionyl chloride and methyl amine as starting materials. MS (ESI (+)) m/z 544 (M+H⁺).

### EXAMPLE 194

### 3-{4-[2-(1-Methyl-piperidin-4-ylamino)-phenylsulfanyl]-2,3-bis-trifluoromethyl-phenyl}-1-morpholin-4-yl-propenone

The procedures for Example 4 utilizing 2-aminothiophenol and Example 8 utilizing N-methyl piperidine as the starting materials were followed. MS (ESI (+)) m/z 556 (M+H⁺).

### EXAMPLE 195

### (4-{3-[4-(3-Morpholin-4-yl-3-oxo-propenyl)-2,3-bis-trifluoromethyl-phenylsulfanyl]-phenylamino}-piperidin-1-yl)-acetic acid

The procedure for Example 172 was followed utilizing chloro-acetic acid as the acyl chloride. MS (ESI (+)) m/z 600 (M+H⁺).

### EXAMPLE 196

### 3-(4-{3-[1-(2-Dimethylamino-acetyl)-azepan-4-ylamino]-phenylsulfanyl}-2,3-bis-trifluoromethyl-phenyl)-1-morpholin-4-yl-propenone

The procedure for Example 179 was followed utilizing 4-oxo-azepane-1-carboxylic acid tert-butyl ester as the starting amine. MS (ESI (+)) m/z 641 (M+H⁺).

### EXAMPLE 197

### 3-{4-[3-(2-Methyl-piperidin-4-ylamino)-phenylsulfanyl]-2,3-bis-trifluoromethyl-phenyl}-1-morpholin-4-yl-propenone

Product in Example 184 was subjected to the procedure described in Example 191. MS (ESI (+)) m/z 556 (M+H⁺).

### EXAMPLE 198

### 2-Cyclopropylamino-N-{3-[4-(3-morpholin-4-yl-3-oxo-propenyl)-2,3-bis-trifluoromethyl-phenylsulfanyl]-phenyl}-acetamide

The product from Example 4 was dissolved in DCM and treated with an excess of diisopropylethyl amine and bromoacetyl chloride. The product from this reaction was further treated with cyclopropyl amine to afford the desired product. MS (ESI (+)) m/z 574 (M+H⁺).

### EXAMPLE 199

### 3-Cyclopropylamino-N-{3-[4-(3-morpholin-4-yl-3-oxo-propenyl)-2,3-bis-trifluoromethyl-phenylsulfanyl]-phenyl}-propionamide

The product from Example 4 was dissolved in DCM and treated with an excess of diisopropylethyl amine and 3-bromopropionyl chloride. The product from this reaction was further treated with cyclopropyl amine to afford the desired product. MS (ESI (+)) m/z 588 (M+H⁺).

### EXAMPLE 200

### 1-(4-Morpholin-4-yl-piperidin-1-yl)-3-{4-[3-(tetrahydro-pyran-4-ylamino)-phenylsulfanyl]-2,3-bis-trifluoromethyl-phenyl}-propenone

The product of Example 233 was subjected to procedure described in Example 219 using 4-piperidin-4-yl-morpholine in place of thiomorpholine to afford the final product. MS (ESI (+)) m/z 644 (M+H⁺).

### EXAMPLE 201

### [1-(3-{4-[3-(Tetrahydro-pyran-4-ylamino)-phenylsulfanyl]-2,3-bis-trifluoromethyl-phenyl}-acryloyl)-piperidin-4-yl]-carbamic acid tert-butyl ester

The product of Example 233 was subjected to procedure described in Example 219 using piperidin-4-yl-carbamic acid tert-butyl ester in place of thiomorpholine to afford the final product. MS (ESI (+)) m/z 674 (M+H⁺).

### EXAMPLE 202

### 1-(4-Dimethylamino-piperidin-1-yl)-3-{4-[3-(tetrahydro-pyran-4-ylamino)-phenylsulfanyl]-2,3-bis-trifluoromethyl-phenyl}-propenone

The product of Example 233 was subjected to procedure described in Example 219 using dimethyl-piperidin-4-yl-amine in place of thiomorpholine to afford the final product. MS (ESI (+)) m/z 602 (M+H⁺).

### EXAMPLE 203

### 1-(4-Acetyl-piperazin-1-yl)-3-{4-[3-(tetrahydro-pyran-4-ylamino)-phenylsulfanyl]-2,3-bis-trifluoromethyl-phenyl}-propenone

The product of Example 233 was subjected to procedure described in Example 219 using 1-piperazin-1-yl-ethanone in place of thiomorpholine to afford the final product. MS (ESI (+)) m/z 602 (M+H⁺).

### EXAMPLE 204

### 1-(4-Amino-piperidin-1-yl)-3-{4-[3-(tetrahydro-pyran-4-ylamino)-phenylsulfanyl]-2,3-bis-trifluoromethyl-phenyl}-propenone

The product of Example 201 was subjected to procedure described in Example 217 to afford the final product. MS (ESI (+)) m/z 574 (M+H⁺).

### EXAMPLE 205

### 2-({3-[4-(3-Morpholin-4-yl-3-oxo-propenyl)-2,3-bis-trifluoromethyl-phenylsulfanyl]-phenylamino}-methyl)-cyclopropanecarboxylic acid

The product of Example 4 was subjected to procedure of Example 17 using 2-formyl-cyclopropanecarboxylic acid ethyl ester in place of tetrahydro-pyran-4-one to prepare 2-({3-[4-(3-morpholin-4-yl-3-oxo-propenyl)-2,3-bis-trifluoromethyl-phenylsulfanyl]-phenylamino}-methyl)-cyclopropanecarboxylic acid ethyl ester. MS (ESI (+)) m/z 603 (M+H⁺). This product was subjected to the procedure described in Example 233 to afford the final product. MS (ESI (+)) m/z 575 (M+H⁺).

### EXAMPLE 206

### 2-Oxo-imidazolidine-1-carboxylic acid {3-[4-(3-morpholin-4-yl-3-oxo-propenyl)-2,3-bis-trifluoromethyl-phenylsulfanyl]-phenyl}-amide

The product of Example 4 was subjected to procedure described in Example 218 using 2-oxo-imidazolidine-1-carbonyl chloride in place of methoxyacetyl chloride to afford the final product. MS (ESI (+)) m/z 589 (M+H⁺).

### EXAMPLE 207

### 1-Morpholin-4-yl-3-(4-{3-[1-(tetrahydro-pyran-4-carbonyl)-piperidin-4-ylamino]-phenylsulfanyl}-2,3-bis-trifluoromethyl-phenyl)-propenone

The product of Example 281 was dissolved in acetonitrile and excess triethylamine was added. Tetrahydro-pyran-4-carboxylic acid (1.2 eq.) and HATU (1.2 eq.) were then added, and after ten minutes the reaction mixture was concentrated. The crude product was extracted from water with ethyl acetate and concentrated, then purified using preparative HPLC to give the final product. MS (ESI (+)) m/z 672 (M+H⁺).

### EXAMPLE 208

### 3-(4-{3-[1-(4-Hydroxy-cyclohexanecarbonyl)-piperidin-4-ylamino]-phenylsulfanyl}-2,3-bis-trifluoromethyl-phenyl)-1-morpholin-4-yl-propenone

The procedure for Example 207 was followed utilizing 4-hydroxy-cyclohexanecarboxylic acid in place of tetrahydro-pyran-4-carboxylic acid. MS (ESI (+)) m/z 686 (M+H⁺).

### EXAMPLE 209

### 1-(4-{3-[4-(3-Morpholin-4-yl-3-oxo-propenyl)-2,3-bis-trifluoromethyl-phenylsulfanyl]-phenylamino}-piperidine-1-carbonyl)-imidazolidin-2-one

The product of Example 281 was subjected to procedure described in Example 207 to afford the final product. MS (ESI (+)) m/z 672 (M+H⁺).

### EXAMPLE 210

### 1-Morpholin-4-yl-3-(4-{3-[1-(tetrahydro-furan-2-carbonyl)-piperidin-4-ylamino]-phenylsulfanyl}-2,3-bis-trifluoromethyl-phenyl)-propenone

The procedure for Example 207 was followed utilizing tetrahydro-furan-2-carboxylic acid in place of 4-hydroxy-cyclohexanecarboxylic acid to afford the final product. MS (ESI (+)) m/z 658 (M+H⁺).

### EXAMPLE 211

### 3-(4-{3-[1-(Morpholine-4-carbonyl)-piperidin-4-ylamino]-phenylsulfanyl}-2,3-bis-trifluoromethyl-phenyl)-1-morpholin-4-yl-propenone

The product of Example 281 was subjected to procedure described in Example 206 using morpholine-4-carbonyl chloride in place of 2-oxo-imidazolidine-1-carbonyl chloride to afford the final product. MS (ESI (+)) m/z 673 (M+H⁺).

### EXAMPLE 212

### 1-Morpholin-4-yl-3-(4-{3-[1-(pyrrolidine-1-carbonyl)-piperidin-4-ylamino]-phenylsulfanyl}-2,3-bis-trifluoromethyl-phenyl)-propenone

The product of Example 281 was subjected to procedure described in Example 206 using pyrrolidine-1-carbonyl chloride in place of 2-oxo-imidazolidine-1-carbonyl chloride to afford the final product. MS (ESI (+)) m/z 657 (M+H⁺).

### EXAMPLE 213

### 4-{3-[4-(3-Morpholin-4-yl-3-oxo-propenyl)-2,3-bis-trifluoromethyl-phenylsulfanyl]-phenylamino}-piperidine-1-carboxylic acid dimethylamide

The product of Example 281 was subjected to procedure described in Example 206 using dimethylamino-1-carbonyl chloride in place of 2-oxo-imidazolidine-1-carbonyl chloride to afford the final product. MS (ESI (+)) m/z 631 (M+H⁺).

### EXAMPLE 214

### 3-{4-[3-(1-Methanesulfonyl-piperidin-4-ylamino)-phenylsulfanyl]-2,3-bis-trifluoromethyl-phenyl}-1-morpholin-4-yl-propenone

The product of Example 281 was subjected to procedure described in Example 206 using methyl sulfonyl chloride in place of 2-oxo-imidazolidine-1-carbonyl chloride to afford the final product. MS (ESI (+)) m/z 638 (M+H⁺).

### EXAMPLE 215

### 4-{3-[4-(3-Morpholin-4-yl-3-oxo-propenyl)-2,3-bis-trifluoromethyl-phenylsulfanyl]-phenylamino}-piperidine-1-carboxylic acid tert-butyl ester

Product of Example 4 was dissolved in dichloroethane to which was added acetic acid and 4 Å molecular sieves. The reaction was heated to 70° C., followed by the addition of 4-oxo-piperidine-1-carboxylic acid tert-butyl ester. After several hours the reaction was cooled to room temperature and sodium triacetoxyborohydride was added in excess. The crude product was purified by flash chromatography to afford the final product. MS (ESI (+)) m/z 660 (M+H⁺).

### EXAMPLE 216

### 4-{3-[4-(2-Carboxy-vinyl)-2,3-bis-trifluoromethyl-phenylsulfanyl]-phenylamino}-piperidine-1-carboxylic acid tert-butyl ester

Product of Example 215 was dissolved in a 1:1 tetrahydrofuran/methanol solution. To this solution, three equivalents of aqueous potassium hydroxide were added and the reaction mixture was heated to 90° C. After sixteen hours the reaction was concentrated and then triturated with aqueous acetic acid to afford the final product. MS (ESI (+)) m/z 591 (M+H⁺).

### EXAMPLE 217

### 3-{4-[3-(Piperidin-4-ylamino)-phenylsulfanyl]-2,3-bis-trifluoromethyl-phenyl}-acrylic acid

Product of Example 216 was dissolved in dichloromethane to which trifluoroacetic acid was added in molar excess. After one hour the reaction was concentrated to give the final product. MS (ESI (+)) m/z 491 (M+H⁺).

### EXAMPLE 218

### 3-(4-{3-[1-(2-Methoxy-acetyl)-piperidin-4-ylamino]-phenylsulfanyl}-2,3-bis-trifluoromethyl-phenyl)-acrylic acid

Product of Example 217 was dissolved in a 1:1 solution of tetrahydrofuran and water. To this solution was added an excess of aqueous potassium carbonate, followed by one equivalent of methoxy-acetyl chloride. After 0.5 hr the reaction was concentrated, extracted with ethyl acetate from water and concentrated to afford the final product. MS (ESI (+)) m/z 563 (M+H⁺).

### EXAMPLE 219

### 3-(4-{3-[1-(2-Methoxy-acetyl)-piperidin-4-ylamino]-phenylsulfanyl}-2,3-bis-trifluoromethyl-phenyl)-1-thiomorpholin-4-yl-propenone

Product of Example 218 was dissolved in acetonitrile and excess triethylamine was added. Thiomorpholine (1.2 eq.) and HATU (1.2 eq.) were then added and after ten minutes the reaction mixture was concentrated. The product was extracted from water with ethyl acetate and concentrated. The crude product was then purified using preparative HPLC to give the final product. MS (ESI (+)) m/z 648 (M+H⁺).

### EXAMPLE 220

### 3-(4-{3-[1-(2-Methoxy-acetyl)-piperidin-4-ylamino]-phenylsulfanyl}-2,3-bis-trifluoromethyl-phenyl)-1-(4-pyridin-2-yl-piperazin-1-yl)-propenone

The procedure for Example 219 was followed utilizing 1-pyridin-2-yl-piperazine in place of thiomorpholine. MS (ESI (+)) m/z 708 (M+H⁺).

### EXAMPLE 221

### 3-(4-{3-[1-(2-Methoxy-acetyl)-piperidin-4-ylamino]-phenylsulfanyl}-2,3-bis-trifluoromethyl-phenyl)-N-(2-methoxy-ethyl)-acrylamide

The procedure for Example 219 was followed utilizing 2-methoxy-ethylamine in place of thiomorpholine. MS (ESI (+)) m/z 620 (M+H⁺).

### EXAMPLE 222

### N-Ethyl-3-(4-{3-[1-(2-methoxy-acetyl)-piperidin-4-ylamino]-phenylsulfanyl}-2,3-bis-trifluoromethyl-phenyl)-N-(2-methoxy-ethyl)-acrylamide

The procedure for Example 219 was followed utilizing 2-ethyl-(2-methoxy-ethyl)-amine in place of thiomorpholine. MS (ESI (+)) m/z 648 (M+H⁺).

### EXAMPLE 223

### 1-(4-Ethanesulfonyl-piperazin-1-yl)-3-(4-{3-[1-(2-methoxy-acetyl)-piperidin-4-ylamino]-phenylsulfanyl}-2,3-bis-trifluoromethyl-phenyl)-propenone

The procedure for Example 219 was followed utilizing 1-ethanesulfonyl-piperazine in place of thiomorpholine. MS (ESI (+)) m/z 723 (M+H⁺).

### EXAMPLE 224

### 1-(3,6-Dihydro-2H-pyridin-1-yl)-3-(4-{3-[1-(2-methoxy-acetyl)-piperidin-4-ylamino]-phenylsulfanyl}-2,3-bis-trifluoromethyl-phenyl)-propenone

The procedure for Example 215 was followed utilizing 1,2,3,6-tetrahydro-pyridine in place of thiomorpholine. MS (ESI (+)) m/z 628 (M+H⁺).

### EXAMPLE 225

### 1-(4-Hydroxy-piperidin-1-yl)-3-(4-{3-[1-(2-methoxy-acetyl)-piperidin-4-ylamino]-phenylsulfanyl}-2,3-bis-trifluoromethyl-phenyl)-propenone

The procedure for Example 219 was followed utilizing piperidin-4-ol in place of thiomorpholine. MS (ESI (+)) m/z 646 (M+H⁺).

### EXAMPLE 226

### 4-[3-(4-{3-[1-(2-Methoxy-acetyl)-piperidin-4-ylamino]-phenylsulfanyl}-2,3-bis-trifluoromethyl-phenyl)-acryloyl]-piperazine-1-carbaldehyde

The procedure for Example 219 was followed utilizing piperazine-1-carbaldehyde in place of thiomorpholine. MS (ESI (+)) m/z 659 (M+H⁺).

### EXAMPLE 227

### 3-(4-{3-[1-(2-Methoxy-acetyl)-piperidin-4-ylamino]-phenylsulfanyl}-2,3-bis-trifluoromethyl-phenyl)-N-(2-methyl-2H-pyrazol-3-yl)-acrylamide

The procedure for Example 219 was followed utilizing 2-methyl-2H-pyrazol-3-ylamine in place of thiomorpholine. MS (ESI (+)) m/z 642 (M+H⁺).

### EXAMPLE 228

### 3-(4-{3-[1-(2-Methoxy-acetyl)-piperidin-4-ylamino]-phenylsulfanyl}-2,3-bis-trifluoromethyl-phenyl)-N-(2-oxo-piperidin-3-yl)-acrylamide

The procedure for Example 219 was followed utilizing 3-amino-piperidin-2-one in place of thiomorpholine. MS (ESI (+)) m/z 659 (M+H⁺).

### EXAMPLE 229

### 3-(4-{3-[1-(2-Methoxy-acetyl)-piperidin-4-ylamino]-phenylsulfanyl}-2,3-bis-trifluoromethyl-phenyl)-1-(2,3,5,6-tetrahydro-[1,2′]bipyrazinyl-4-yl)-propenone

The procedure for Example 219 was followed utilizing 3,4,5,6-tetrahydro-2H-[1,2′]bipyrazinyl in place of thiomorpholine. MS (ESI (+)) m/z 709 (M+H⁺).

### EXAMPLE 230

### {1-[3-(4-{3-[1-(2-Methoxy-acetyl)-piperidin-4-ylamino]-phenylsulfanyl}-2,3-bis-trifluoromethyl-phenyl)-acryloyl]-piperidin-4-yl}-acetic acid ethyl ester

The procedure for Example 219 was followed utilizing piperidin-4-yl-acetic acid ethyl ester in place of thiomorpholine. MS (ESI (+)) m/z 716 (M+H⁺).

### EXAMPLE 231

### {1-[3-(4-{3-[1-(2-Methoxy-acetyl)-piperidin-4-ylamino]-phenylsulfanyl}-2,3-bis-trifluoromethyl-phenyl)-acryloyl]-piperidin-4-yl}-acetic acid

Example 230 was dissolved in tetrahydrofuran and a few drops of methanol to which was added excess aqueous lithium hydroxide. After two hours the reaction was concentrated and triturated with aqueous acetic acid to afford the final product. MS (ESI (+)) m/z 688 (M+H⁺).

### EXAMPLE 232

### 2-{1-[3-(4-{3-[1-(2-Methoxy-acetyl)-piperidin-4-ylamino]-phenylsulfanyl}-2,3-bis-trifluoromethyl-phenyl)-acryloyl]-piperidin-4-yl}-N,N-dimethyl-acetamide

The procedure for Example 219 was followed utilizing dimethyl-amine in place of thiomorpholine. MS (ESI (+)) m/z 715 (M+H⁺).

### EXAMPLE 233

### 3-{4-[3-(Tetrahydro-pyran-4-ylamino)-phenylsulfanyl]-2,3-bis-trifluoromethyl-phenyl}-acrylic acid

The procedure for 216 was followed utilizing Example 17 in place of Example 215. MS (ESI (+)) m/z 492 (M+H⁺).

### EXAMPLE 234

### 3 1-(4-Pyridin-2-yl-piperazin-1-yl)-3-{4-[3-(tetrahydro-pyran-4-ylamino)-phenylsulfanyl]-2,3-bis-trifluoromethyl-phenyl}-propenone

The procedure for Example 219 was followed utilizing 1-pyridin-2-yl-piperazine in place of thiomorpholine. MS (ESI (+)) m/z 637 (M+H⁺).

### EXAMPLE 235

### 1-(3,6-Dihydro-2H-pyridin-1-yl)-3-{4-[3-(tetrahydro-pyran-4-ylamino)-phenylsulfanyl]-2,3-bis-trifluoromethyl-phenyl}-propenone

The procedure for Example 219 was followed utilizing 1,2,3,6-tetrahydro-pyridine in place of thiomorpholine. MS (ESI (+)) m/z 557 (M+H⁺).

### EXAMPLE 236

### 1-(4-Ethanesulfonyl-piperazin-1-yl)-3-{4-[3-(tetrahydro-pyran-4-ylamino)-phenylsulfanyl]-2,3-bis-trifluoromethyl-phenyl}-propenone

The procedure for Example 219 was followed utilizing 1-ethanesulfonyl-piperazine in place of thiomorpholine. MS (ESI (+)) m/z 652 (M+H⁺).

### EXAMPLE 237

### 1-(4-Hydroxy-piperidin-1-yl)-3-{4-[3-(tetrahydro-pyran-4-ylamino)-phenylsulfanyl]-2,3-bis-trifluoromethyl-phenyl}-propenone

The procedure for Example 219 was followed utilizing piperidin-4-ol in place of thiomorpholine. MS (ESI (+)) m/z 575 (M+H⁺).

### EXAMPLE 238

### 1-(2,3,5,6-Tetrahydro-[1,2′]bipyrazinyl-4-yl)-3-{4-[3-(tetrahydro-pyran-4-ylamino)-phenylsulfanyl]-2,3-bis-trifluoromethyl-phenyl}-propenone

The procedure for Example 219 was followed utilizing 3,4,5,6-tetrahydro-2H-[1,2′]bipyrazinyl in place of thiomorpholine. MS (ESI (+)) m/z 638 (M+H⁺).

### EXAMPLE 239

### [1-(3-{4-[3-(Tetrahydro-pyran-4-ylamino)-phenylsulfanyl]-2,3-bis-trifluoromethyl-phenyl}-acryloyl)-piperidin-4-yl]-acetic acid ethyl ester

The procedure for Example 219 was followed utilizing piperidin-4-yl-acetic acid ethyl ester in place of thiomorpholine. MS (ESI (+)) m/z 645 (M+H⁺).

### EXAMPLE 240

### [1-(3-{4-[3-(Tetrahydro-pyran-4-ylamino)-phenylsulfanyl]-2,3-bis-trifluoromethyl-phenyl}-acryloyl)-piperidin-4-yl]-acetic acid

The procedure for Example 231 was followed using Example 242 in place of Example 230 to afford the product. MS (ESI (+)) m/z 617 (M+H⁺).

### EXAMPLE 241

### N,N-Dimethyl-2-[1-(3-{4-[3-(tetrahydro-pyran-4-ylamino)-phenylsulfanyl]-2,3-bis-trifluoromethyl-phenyl}-acryloyl)-piperidin-4-yl]-acetamide

The procedure for Example 219 was followed utilizing the product of Example 240 and dimethyl-amine in place of thiomorpholine. MS (ESI (+)) m/z 644 (M+H⁺).

### EXAMPLE 242

### 4-({3-[4-(3-Morpholin-4-yl-3-oxo-propenyl)-2,3-bis-trifluoromethyl-phenylsulfanyl]-phenylamino}-methyl)-piperidine-1-carboxylic acid tert-butyl ester

The procedure for Example 17 was followed utilizing 4-formyl-piperidine-1-carboxylic acid tert-butyl ester in place of tetrahydro-pyran-4-one. The crude product was purified by flash chromatography. MS (ESI (+)) m/z 674 (M+H⁺).

### EXAMPLE 243

### 3-(4-{3-[(1-Acetyl-piperidin-4-ylmethyl)-amino]-phenylsulfanyl}-2,3-bis-trifluoromethyl-phenyl)-1-morpholin-4-yl-propenone

The product from Example 242 was dissolved in dichloromethane to which trifluoroacetic acid was added in molar excess. After one hour the reaction was concentrated to give the secondary amine product. The procedure for Example 220 was then followed, substituting acetyl chloride in place of methoxy-acetyl chloride. MS (ESI (+)) m/z 616 (M+H⁺).

### EXAMPLE 244

### 3-{3-[4-(3-Morpholin-4-yl-3-oxo-propenyl)-2,3-bis-trifluoromethyl-phenylsulfanyl]-phenylamino}-pyrrolidine-1-carboxylic acid tert-butyl ester

The procedure for Example 17 was followed utilizing 3-oxo-pyrrolidine-1-carboxylic acid tert-butyl ester in place of tetrahydro-pyran-4-one. The crude product was purified by flash chromatography. MS (ESI (+)) m/z 646 (M+H⁺)

### EXAMPLE 245

### 1-Morpholin-4-yl-3-{4-[3-(pyrrolidin-3-ylamino)-phenylsulfanyl]-2,3-bis-trifluoromethyl-phenyl}propenone

The procedure for Example 217 was followed substituting Example 244 for Example 218. MS (ESI (+)) m/z 546 (M+H⁺).

### EXAMPLE 246

### 3-{4-[3-(1-Acetyl-pyrrolidin-3-ylamino)-phenylsulfanyl]-2,3-bis-trifluoromethyl-phenyl}-1-morpholin-4-yl-propenone

The procedure for Example 22 was followed replacing Example 245 for Example 217 and substituting acetyl chloride in place of methoxy-acetyl chloride. MS (ESI (+)) m/z 588 (M+H⁺).

### EXAMPLE 247

### 1-Methyl-1H-imidazole-2-carboxylic acid {3-[4-(3-morpholin-4-yl-3-oxo-propenyl)-2,3-bis-trifluoromethyl-phenylsulfanyl]-phenyl}-amide

The procedure for Example 219 was followed utilizing 1-methyl-1H-imidazole-2-carboxylic acid in place of thiomorpholine. MS (ESI (+)) m/z 585 (M+H⁺).

### EXAMPLE 248

### 1-Methyl-1H-pyrazole-3-carboxylic acid {3-[4-(3-morpholin-4-yl-3-oxo-propenyl)-2,3-bis-trifluoromethyl-phenylsulfanyl]-phenyl}-amide

The procedure for Example 219 was followed utilizing 1-methyl-1H-pyrazole-3-carboxylic acid in place of thiomorpholine. MS (ESI (+)) m/z 585 (M+H⁺).

### EXAMPLE 249

### 1,5-Dimethyl-1H-pyrazole-3-carboxylic acid {3-[4-(3-morpholin-4-yl-3-oxo-propenyl)-2,3-bis-trifluoromethyl-phenylsulfanyl]-phenyl}-amide

The procedure for Example 219 was followed utilizing 1,5-dimethyl-1H-pyrazole-3-carboxylic acid in place of thiomorpholine. MS (ESI (+)) m/z 599 (M+H⁺).

### EXAMPLE 250

### Pyrimidine-5-carboxylic acid {3-[4-(3-morpholin-4-yl-3-oxo-propenyl)-2,3-bis-trifluoromethyl-phenylsulfanyl]-phenyl}-amide

The procedure for Example 219 was followed utilizing pyrimidine-5-carboxylic acid in place of thiomorpholine. MS (ESI (+)) m/z 583 (M+H⁺).

### EXAMPLE 251

### Pyrazine-2-carboxylic acid {3-[4-(3-morpholin-4-yl-3-oxo-propenyl)-2,3-bis-trifluoromethyl-phenylsulfanyl]-phenyl}-amide

The procedure for Example 219 was followed utilizing pyrazine-2-carboxylic acid in place of thiomorpholine. MS (ESI (+)) m/z 583 (M+H⁺).

### EXAMPLE 252

### 1,1-Dimethyl-3-{3-[4-(3-morpholin-4-yl-3-oxo-propenyl)-2,3-bis-trifluoromethyl-phenylsulfanyl]-phenyl}-urea

Product of Example 4 was dissolved in minimal acetonitrile to which was added excess triethylamine and a catalytic amount of dimethyl-pyridin-4-yl-amine (DMAP) was added. The reaction was heated to 140° C. at which point dimethylcarbamoyl chloride was added in great excess. After ten minutes the reaction was cooled and concentrated. The product was extracted from water with ethyl acetate and concentrated. The crude product was purified by preparative HPLC to afford the final product. MS (ESI (+)) m/z 548 (M+H⁺).

### EXAMPLE 253

### 3-(4-{3-[1-(2-Dimethylamino-acetyl)-piperidin-4-ylamino]-phenylsulfanyl}-2,3-bis-trifluoromethyl-phenyl)-acrylic acid

Product of Example 217 was dissolved in dichloromethane and excess N,N′-diisopropylethylamine (DIEA) was added, followed by addition of dimethylamino-acetyl chloride. After ten minutes the reaction mixture washed with water and the organic layer concentrated. MS (ESI (+)) m/z 576 (M+H⁺).

### EXAMPLE 254

### 3-(4-{3-[1-(2-Dimethylamino-acetyl)-piperidin-4-ylamino]-phenylsulfanyl}-2,3-bis-trifluoromethyl-phenyl)-1-piperidin-1-yl-propenone

The product of Example 253 was subjected to the procedure for Example 219, utilizing piperidine in place of thiomorpholine. MS (ESI (+)) m/z 643 (M+H⁺).

### EXAMPLE 255

### 3-{4-[3-(1-Acetyl-piperidin-4-ylamino)-phenylsulfanyl]-2,3-bis-trifluoromethyl-phenyl}-acrylic acid

The procedure for Example 218 was followed utilizing acetyl chloride in place of methoxyacetyl chloride and Example 217 as the starting material. MS (ESI (+)) m/z 533 (M+H⁺).

### EXAMPLE 256

### 1-(4-Acetyl-piperazin-1-yl)-3-{4-[3-(1-acetyl-piperidin-4-ylamino)-phenylsulfanyl]-2,3-bis-trifluoromethyl-phenyl}-propenone

The procedure for Example 219 was followed utilizing 1-piperazin-1-yl-ethanone in place of thiomorpholine. MS (ESI (+)) m/z 643 (M+H⁺).

### EXAMPLE 257

### 1-Methyl-4-{3-[4-(3-morpholin-4-yl-3-oxo-propenyl)-2,3-bis-trifluoromethyl-phenylsulfanyl]-phenylamino}-piperidine-4-carbonitrile-2,3-bis-trifluoromethyl-phenyl)-N-(2-methoxy-ethyl)-acrylamide

The procedure for Example 263 was followed utilizing 1-methyl-piperidin-4-one in place of tetrahydro-pyran-4-one. MS (ESI (+)) m/z 599 (M+H⁺).

### EXAMPLE 258

### 1-Methyl-4-{3-[4-(3-morpholin-4-yl-3-oxo-propenyl)-2,3-bis-trifluoromethyl-phenylsulfanyl]-phenylamino}-piperidine-4-carboxylic acid amide

The procedure for Example 264 was followed utilizing the product of Example 261 to afford the final product. MS (ESI (+)) m/z 617 (M+H⁺).

### EXAMPLE 259

### (3-{3-[4-(3-Morpholin-4-yl-3-oxo-propenyl)-2,3-bis-trifluoromethyl-phenylsulfanyl]-phenyl}-ureido)-acetic acid ethyl ester

Product of Example 4 was reacted with isocyanato-acetic acid ethyl ester in acetonitrile solvent to afford the crude product that was purified by HPLC. MS ESI (+) m/z 606 (M+H⁺).

### EXAMPLE 260

### Tetrahydro-pyran-4-carboxylic acid {3-[4-(3-morpholin-4-yl-3-oxo-propenyl)-2,3-bis-trifluoromethyl-phenylsulfanyl]-phenyl}-amide

Product of Example 4 was reacted with potassium carbonate and tetrahydro-pyran-4-carbonyl chloride (prepared from tetrahydro-pyran-4-carboxylic acid and thionyl chloride in tetrahydrofuran) to afford the crude product that was purified by trituration with methanol to afford the final product. MS ESI (+) m/z 589 (M+H⁺).

### EXAMPLE 261

### 3-(4-{3-[2-(3-Fluoro-phenyl)-2-oxo-ethylamino]-phenylsulfanyl}-2,3-bis-trifluoromethyl-phenyl)-1-morpholin-4-yl-propenone

Product of Example 4 was reacted with 2-bromo-1-(3-fluoro-phenyl)-ethanone in dioxane solvent at 108° C. for 3 h to afford the product that was purified by flash chromatography to afford the final product. MS ESI (+) m/z 613 (M+H⁺).

### EXAMPLE 262

### 3-(4-{3-[2-(3-Fluoro-phenyl)-2-hydroxy-ethylamino]-phenylsulfanyl}-2,3-bis-trifluoromethyl-phenyl)-1-morpholin-4-yl-propenone

Product of Example 261 was reacted with NaBH₄ in THF to afford the final product that was purified by HPLC. MS ESI (+) m/z 615 (M+H⁺).

### EXAMPLE 263

### 4-{3-[4-(3-Morpholin-4-yl-3-oxo-propenyl)-2,3-bis-trifluoromethyl-phenylsulfanyl]-phenylamino}-tetrahydro-pyran-4-carbonitrile

Product of Example 4 was reacted with tetrahydro-pyran-4-one and potassium cyamide in acetic acid at room temperature for 1 h to afford final product, purified by trituration in MeOH. MS ESI (+) m/z 586 (M+H⁺).

### EXAMPLE 264

### 4-{3-[4-(3-Morpholin-4-yl-3-oxo-Propenyl)-2,3-bis-trifluoromethyl-phenylsulfanyl]-phenylamino}-tetrahydro-pyran-4-carboxylic acid amide

Product of Example 263 was reacted with concentrated sulfuric acid at room temperature for 24 h, followed by neutralization with ammonium hydroxide, and purified by trituration using MeOH to afford the final product. MS ESI (+) m/z 604 (M+H⁺).

### EXAMPLE 265

### 2,3-Dihydroxy-N-{3-[4-(3-morpholin-4-yl-3-oxo-propenyl)-2,3-bis-trifluoromethyl-phenylsulfanyl]-phenyl}-propionamide

2,2-Dimethyl-[1,3]dioxolane-4-carboxylic acid chloride was prepared from 2,2-dimethyl-[1,3]dioxolane-4-carboxylic acid potassium salt and oxalyl chloride (2M in dichloromethane), followed by reaction with the product of Example 4 using potassium carbonate. Subsequent reaction with trifluoroacetic acid at room temp. afforded the product that was purified by HPLC to afford the final product. MS ESI (+) m/z 565 (M+H⁺).

### EXAMPLE 266

### 3-Hydroxy-N-{3-[4-(3-morpholin-4-yl-3-oxo-propenyl)-2,3-bis-trifluoromethyl-phenylsulfanyl]-phenyl}-propionamide

3-Hydroxy-propionyl chloride was prepared from 3-hydroxy-propionic acid using oxalyl chloride (2M in CH₂Cl₂) and reacted with the product of Example 4 using potassium carbonate to afford the final product after HPLC purification. MS ESI (+) m/z 549 (M+H⁺).

### EXAMPLE 267

### 3-(4-{3-[1-(2,3-Dihydroxy-propionyl)-piperidin-4-ylamino]-phenylsulfanyl}-2,3-bis-trifluoromethyl-phenyl)-1-morpholin-4-yl-propenone

The procedure for Example 172 was followed utilizing 2,2-dimethyl-[1,3]dioxolane-4-carboxylic acid potassium salt to afford the final product. MS (ESI (+)) m/z 648 (M+H⁺).

### EXAMPLE 268

### N-{3-[4-(3-Morpholin-4-yl-3-oxo-propenyl)-2,3-bis-trifluoromethyl-phenylsulfanyl]-phenyl}-1-oxy-isonicotinamide

Product of Example 95 was reacted with m-chloroperbenzoic acid in dioxane at room temperature for 24 h to afford the final product after HPLC purification. MS ESI (+) m/z 598 (M+H⁺).

### EXAMPLE 269

### 3-(4-{3-[1-(3-Hydroxy-propionyl)-piperidin-4-ylamino]-phenylsulfanyl}-2,3-bis-trifluoromethyl-phenyl)-1-morpholin-4-yl-propenone

The procedure for Example 172 was followed utilizing 3-hydroxy-propionyl chloride as the acyl chloride (prepared from 3-hydroxypropionic acid and oxalyl chloride) to afford the final product. MS (ESI (+)) m/z 632 (M+H⁺).

### EXAMPLE 270

### 3-(4-{3-[1-(2-Hydroxy-acetyl)-piperidin-4-ylamino]-phenylsulfanyl}-2,3-bis-trifluoromethyl-phenyl)-1-morpholin-4-yl-propenone

The procedure for Example 172 was followed utilizing hydroxyacetyl chloride as the acyl chloride (prepared from hydroxyacetic acid and oxalyl chloride) to afford the final product. MS (ESI (+)) m/z 618 (M+H⁺).

### EXAMPLE 271

### (4-{3-[4-(3-Morpholin-4-yl-3-oxo-propenyl)-2,3-bis-trifluoromethyl-phenylsulfanyl]-phenylamino}-piperidin-1-yl)-acetic acid isopropyl ester

The procedure for Example 172 was followed utilizing bromo-acetic acid isopropyl ester to afford the final product. MS (ESI (+)) m/z 660 (M+H⁺).

### EXAMPLE 272

### (4-{3-[4-(3-Morpholin-4-yl-3-oxo-propenyl)-2,3-bis-trifluoromethyl-phenylsulfanyl]-phenylamino}-piperidin-1-yl)-acetic acid tert-butyl ester

The procedure for Example 172 was followed utilizing bromo-acetic acid tert-butyl ester to afford the final product. MS (ESI (+)) m/z 674 (M+H⁺).

### EXAMPLE 273

### 6-{3-[4-(3-Morpholin-4-yl-3-oxo-propenyl)-2,3-bis-trifluoromethyl-phenylsulfanyl]-phenylamino}-1H-pyrimidine-2,4-dione

The product of Example 4 was treated with 6-chlorouracil and heated over a period of 5 min. to afford a crude product that was subjected to HPLC purification to give the final product. MS ESI (+) m/z 587 (M+H⁺).

### EXAMPLE 274

### N-{2-[4-(3-Morpholin-4-yl-3-oxo-propenyl)-2,3-bis-trifluoromethyl-phenylsulfanyl]-phenyl}-2-piperidin-1-yl-acetamide

3-[4-(2-Amino-phenylsulfanyl)-2,3-bis-trifluoromethyl-phenyl]-1-morpholin-4-yl-propenone, an intermediate produced in Example 194 was subjected to the procedure described in Example 193 utilizing piperidine in place of methyl amine to afford the final product after HPLC purification. MS ESI (+) m/z 602 (M+H⁺).

### EXAMPLE 275

### (2-{4-[3-(1-Methyl-piperidin-4-ylamino)-phenylsulfanyl]-2,3-bis-trifluoromethyl-phenyl}-cyclopropyl)-morpholin-4-yl-methanone

The product of Example 18 is treated with a solution of trimethylsulfoxonium iodide in DMSO in the presence of NaH. The crude product is subjected to HPLC purification to give the final product.

### EXAMPLE 276

### (2-{4-[2-(1-Methyl-piperidin-4-ylamino)-phenylsulfanyl]-2,3-bis-trifluoromethyl-phenyl}-cyclopropyl)-morpholin-4-yl-methanone

The product of Example 194 is treated with a solution of trimethylsulfoxonium iodide in DMSO in the presence of NaH. The crude product is subjected to HPLC purification to give the final product.

### EXAMPLE 277

### (1-Methyl-piperidin-4-yl)-{3-[4-(2-morpholin-4-yl-pyridin-4-yl)-2,3-bis-trifluoromethyl-phenylsulfanyl]-phenyl}-amine

The procedure for Example 278 is followed utilizing 3-amino-benzenethiol instead of 2-amino-benzenethiol to afford the final product.

### EXAMPLE 278

### (1-Methyl-piperidin-4-yl)-{2-[4-(2-morpholin-4-yl-pyridin-4-yl)-2,3-bis-trifluoromethyl-phenylsulfanyl]-phenyl}-amine

### A. [2-(4-Iodo-2,3-bis-trifluoromethyl-phenylsulfanyl)-phenyl]-(1-methyl-piperidin-4-yl)-amine

The procedures for Examples 3, 4 and 18 are followed, utilizing 4-iodo-2,3-bis-trifluoromethyl-phenol (prepared according to the procedure described in Zhu et al., Organic Letters 2:3345-3348 (2000)) instead of the product of Example 2, to afford the final product.

### B. {2-[4-(2-Chloro-pyridin-4-yl)-2,3-bis-trifluoromethyl-phenylsulfanyl]-phenyl}-(1-methyl-piperidin-4-yl)-amine

The product of Example 278A is treated with 4-pyridineboronic acid (1 eq.) in DMF in the presence of a catalytic amount of Pd(0). The reaction mixture is refluxed overnight to give the crude product, which is purified by flash chromatography. The product is then treated with MCPBA in methylene chloride, followed by treatment with POCl₃ to afford the final product, which is purified by flash chromatography.

### C. (1-Methyl-piperidin-4-yl)-{2-[4-(2-morpholin-4-yl-pyridin-4-yl)-2,3-bis-trifluoromethyl-phenylsulfanyl]-phenyl}-amine

The product of Example 278B is heated in DMF in the presence of base, such as sodium hydroxide, and morpholine to afford the final product after HPLC purification.

### EXAMPLE 279

### (1-Methyl-piperidin-4-yl)-{3-[4-(2-morpholin-4-yl-pyridin-4-yl)-2,3-bis-trifluoromethyl-phenylsulfanyl]-phenyl}-amine

The procedure for Example 280 is followed utilizing [3-(4-iodo-2,3-bis-trifluoromethyl-phenylsulfanyl)-phenyl]-(1-methyl-piperidin-4-yl)-amine instead of [2-(4-iodo-2,3-bis-trifluoromethyl-phenylsulfanyl)-phenyl]-(1-methyl-piperidin-4-yl)-amine to afford the final product.

### EXAMPLE 280

### (1-Methyl-piperidin-4-yl)-{2-[4-(6-morpholin-4-yl-pyrimidin-4-yl)-2,3-bis-trifluoromethyl-phenylsulfanyl]-phenyl}-amine

### A. {2-[4-(6-Iodo-pyrimidin-4-yl)-2,3-bis-trifluoromethyl-phenylsulfanyl]-phenyl}-(1-methyl-piperidin-4-yl)-amine

The product of Example 278A in THF is treated consecutively with butyl lithium, zinc chloride, triphenyl phosphine and a catalytic amount of palladium catalyst, followed by the addition of 4,6-diiodo-pyrimidine. The reaction mixture is refluxed overnight to give the crude product, which is purified by flash chromatography to afford the final product.

### B. (1-Methyl-piperidin-4-yl)-{2-[4-(6-morpholin-4-yl-pyrimidin-4-yl)-2,3-bis-trifluoromethyl-phenylsulfanyl]-phenyl}-amine

The product of Example 280A is subjected to the procedure described in Example 278C to afford the final product after HPLC purification.

### EXAMPLE 281

### 1-Morpholin-4-yl-3-{4-[3-(Piperidin-4-ylamino)-phenylsulfanyl]-2,3-bis-trifluoromethyl-phenyl}-propenone

The product of Example 215 was subjected to the procedure described in Example 217 to afford the final product after HPLC purification. MS ESI (+) m/z 560 (M+H⁺).

The structure of the product compound obtained in each example is given below.

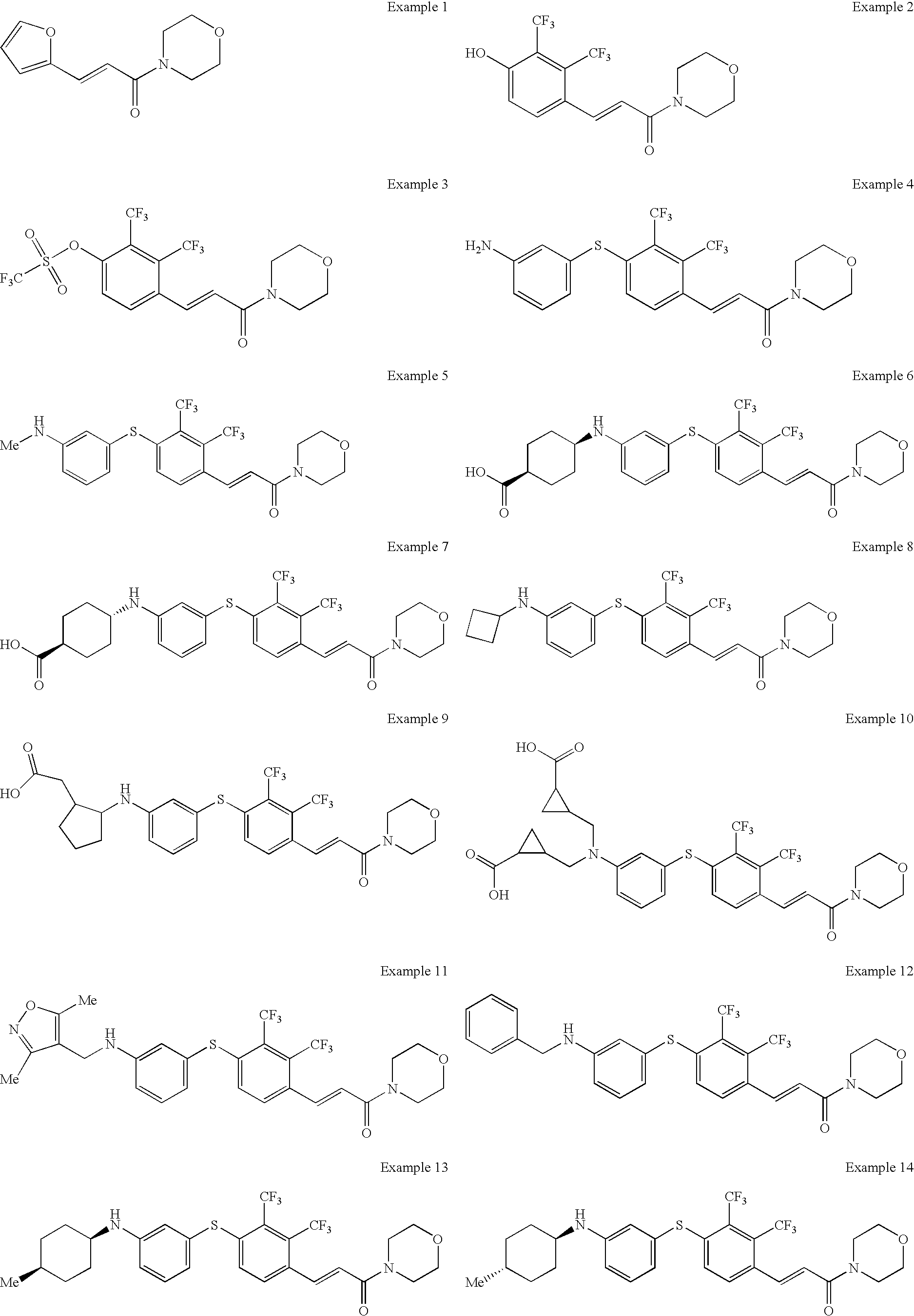

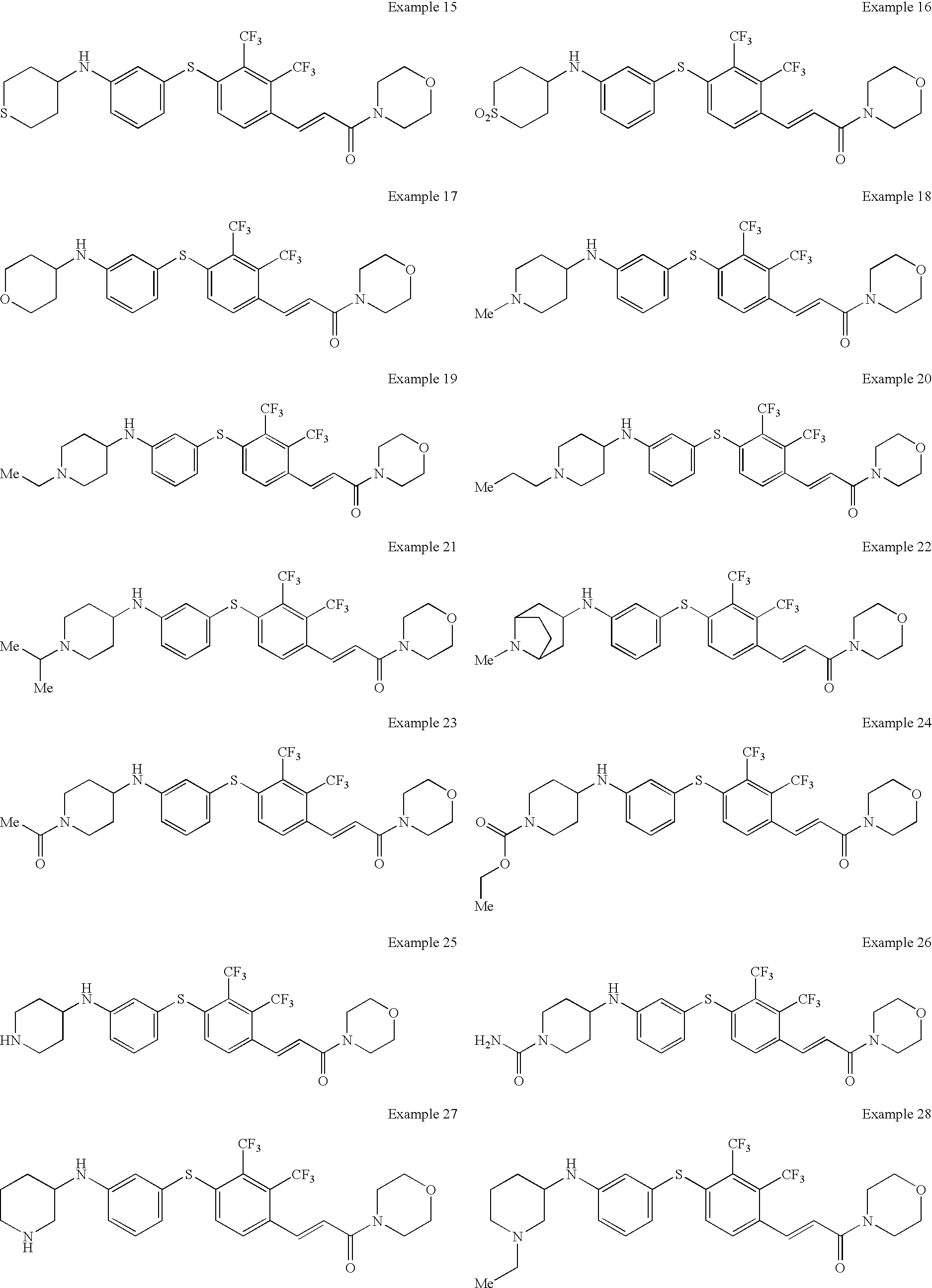

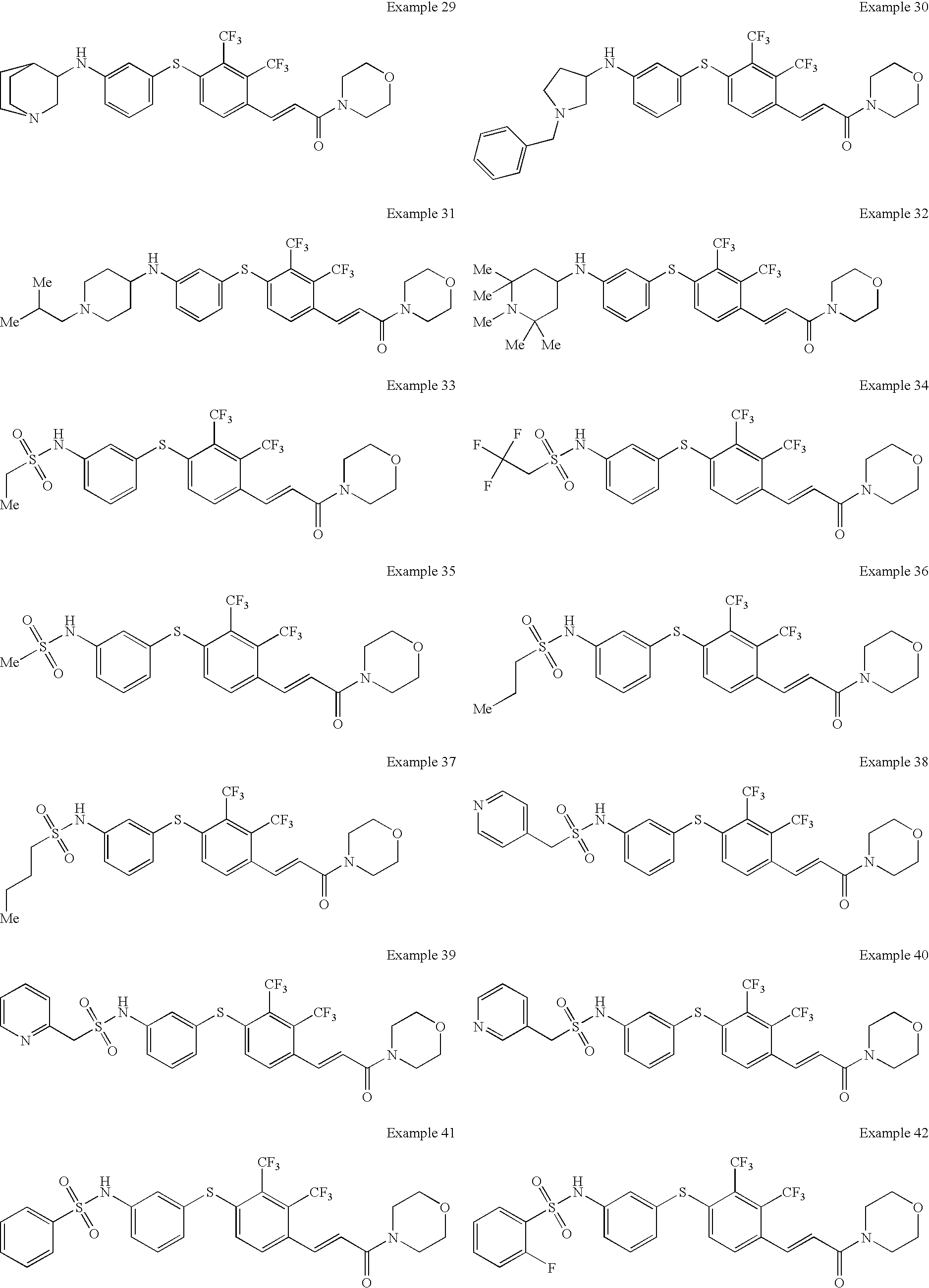

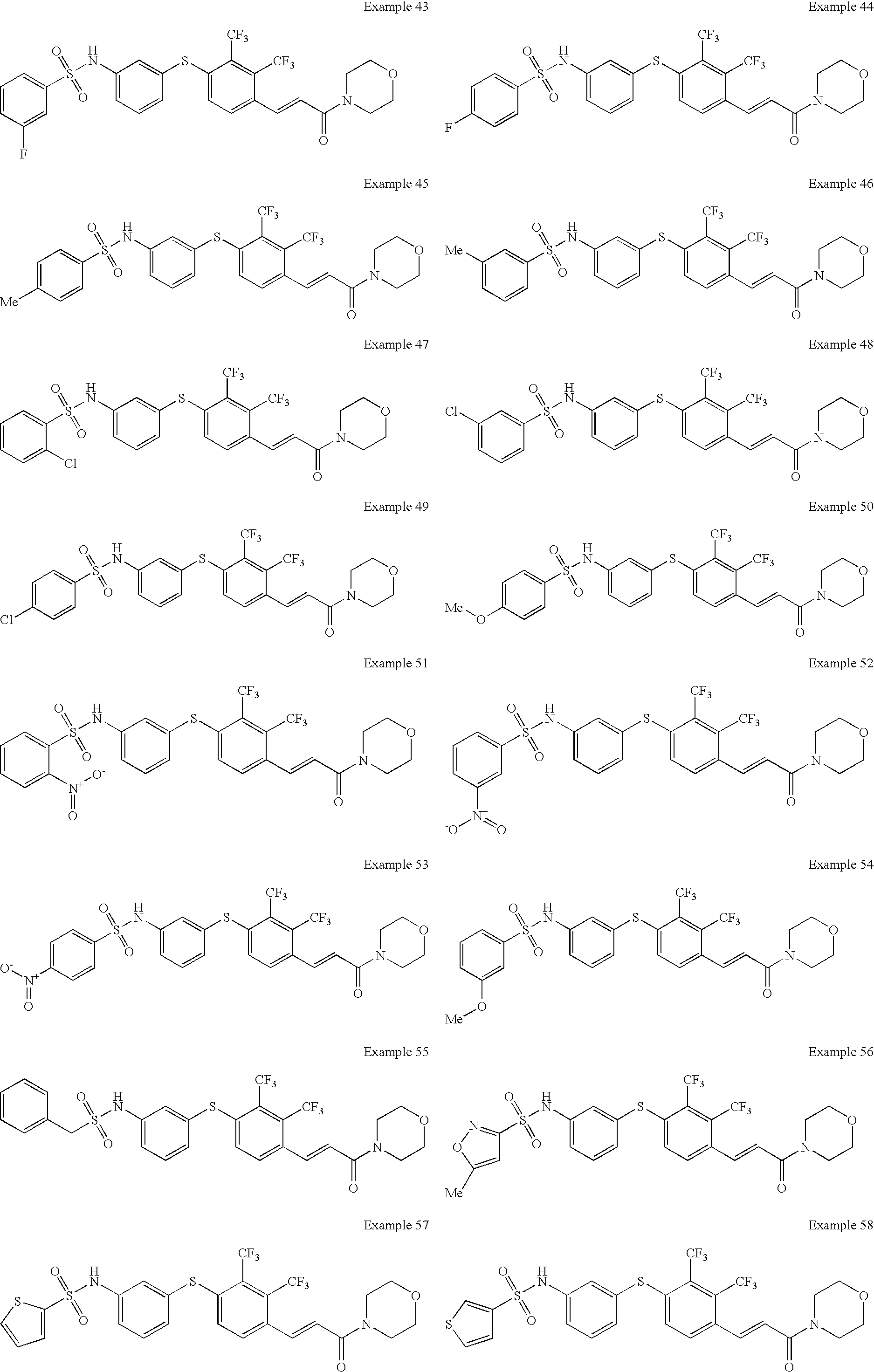

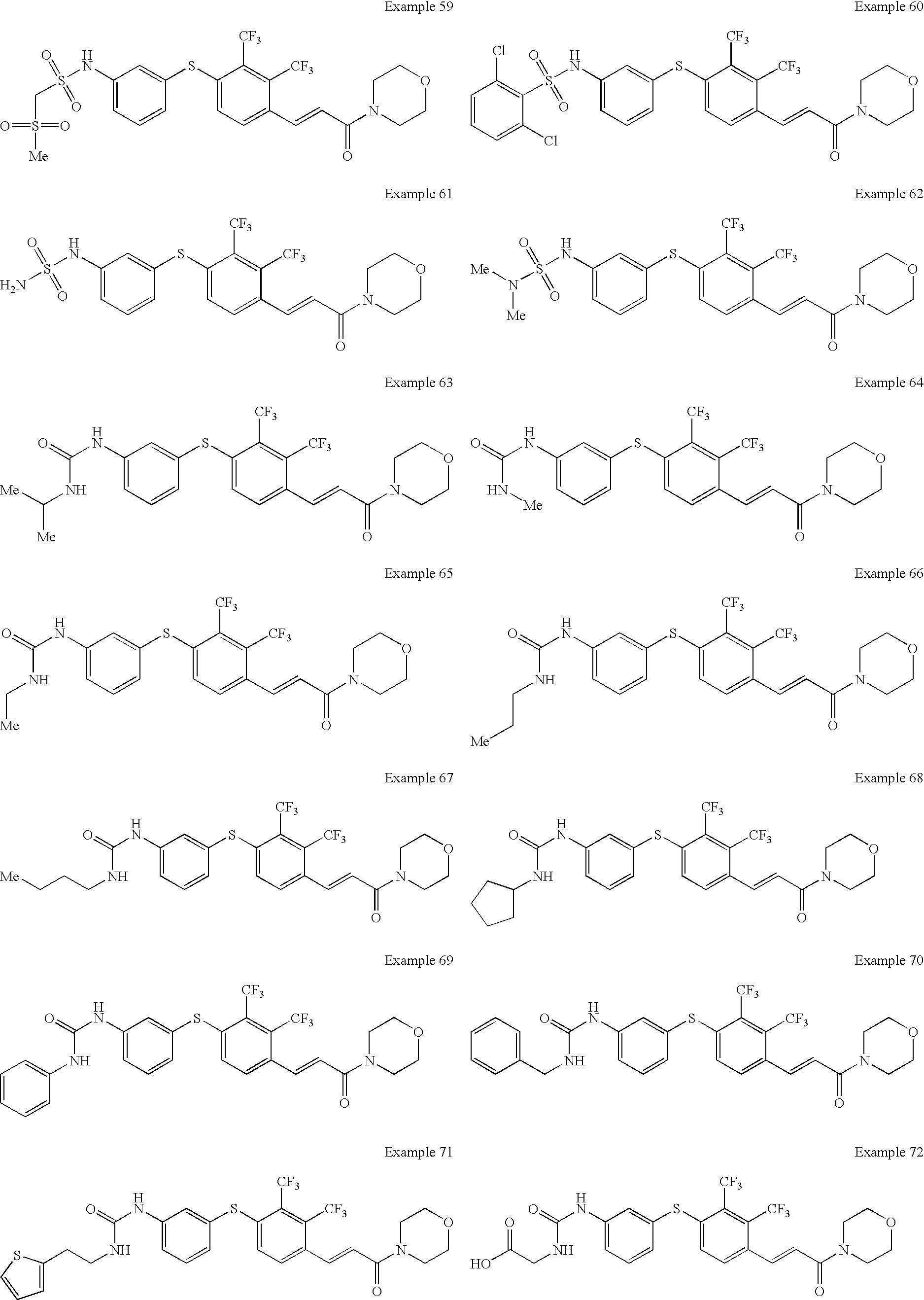

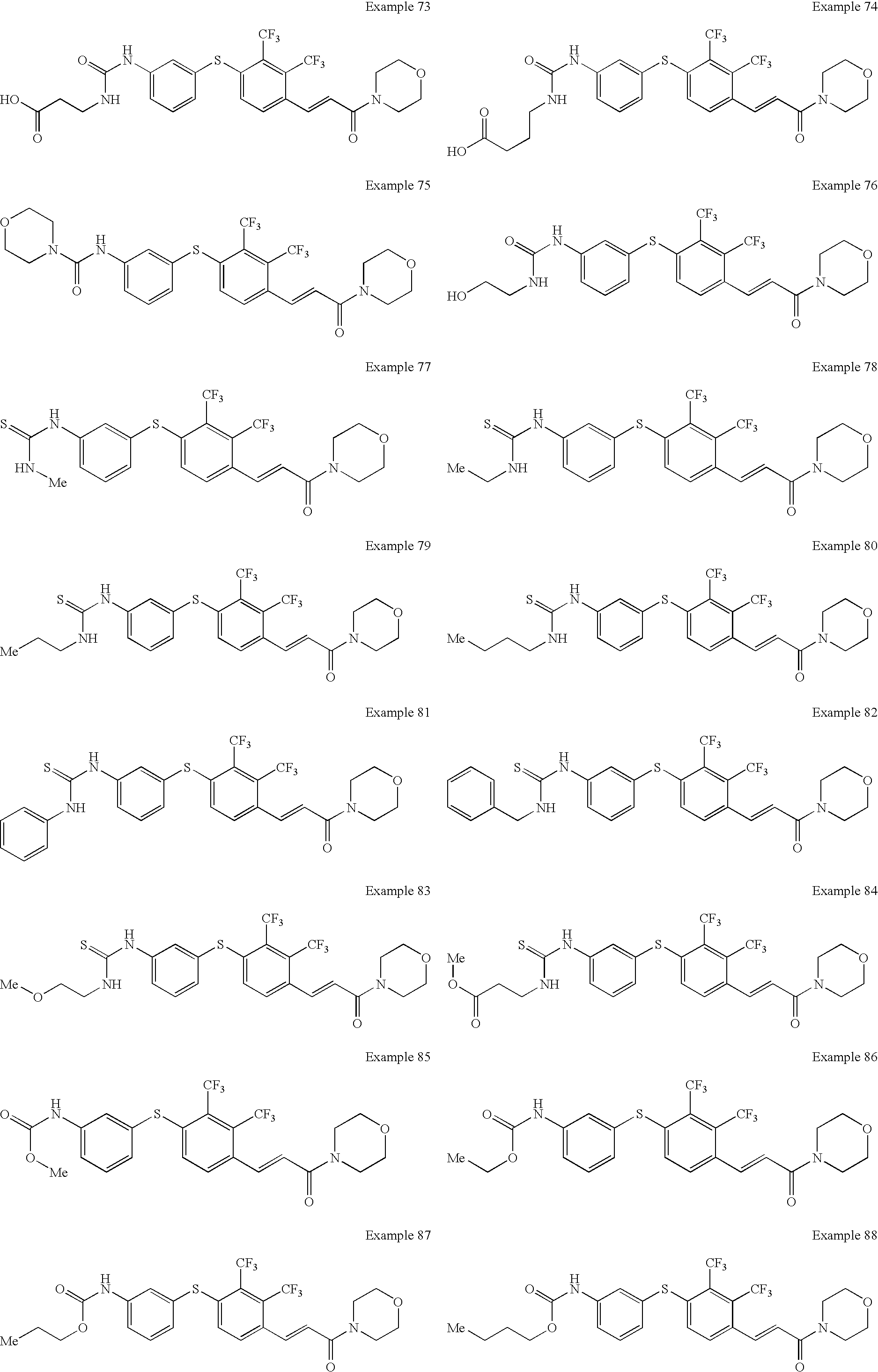

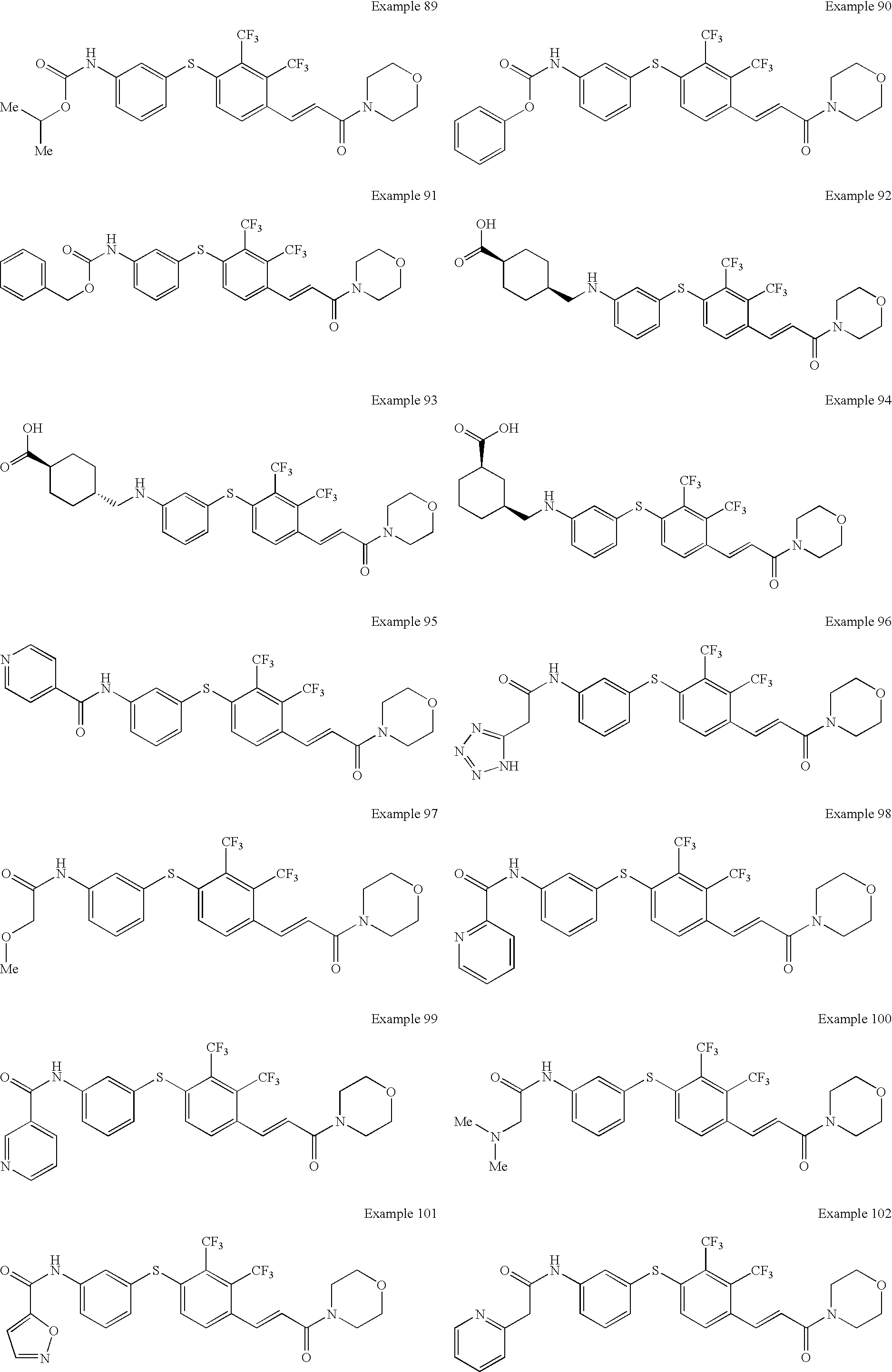

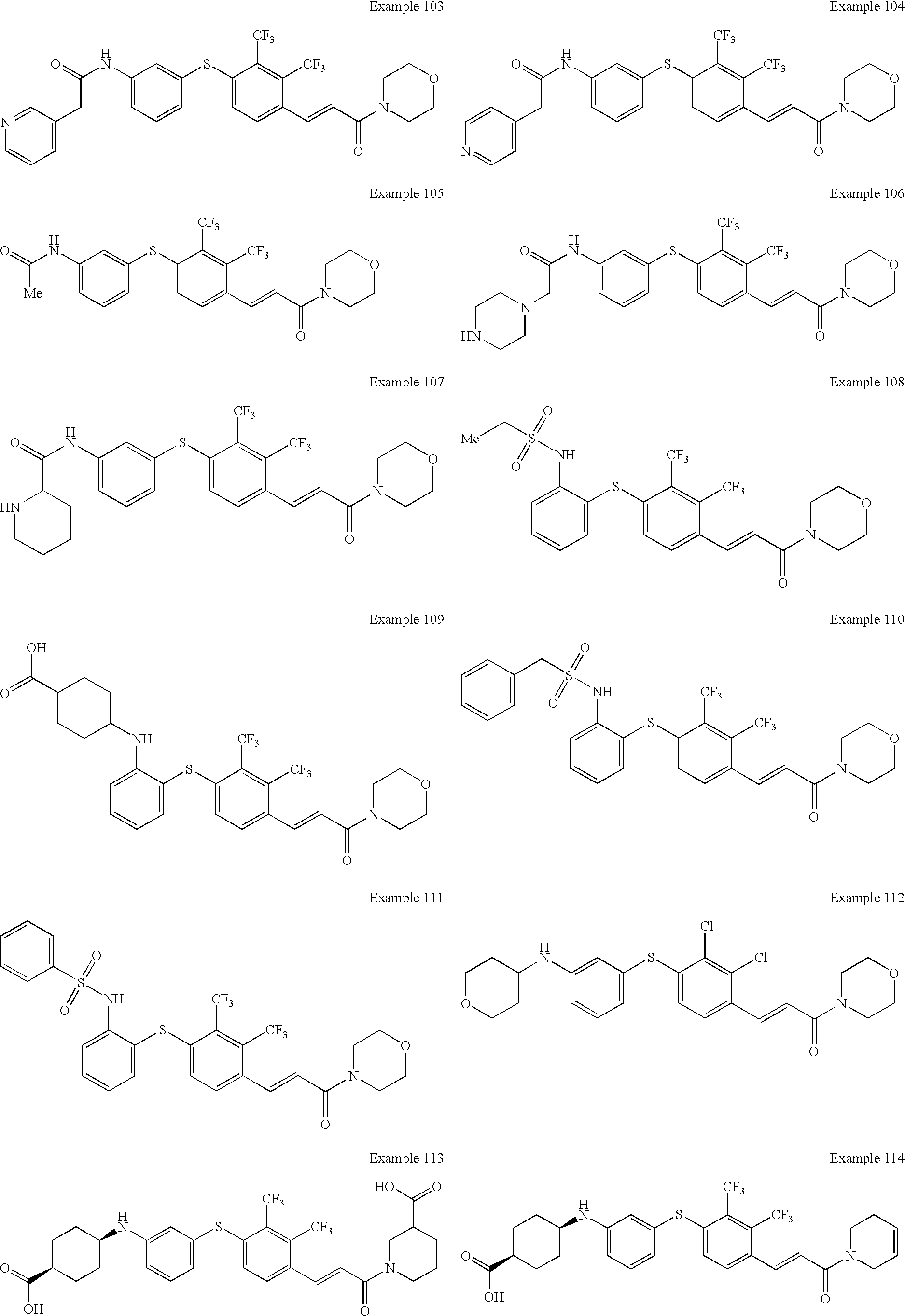

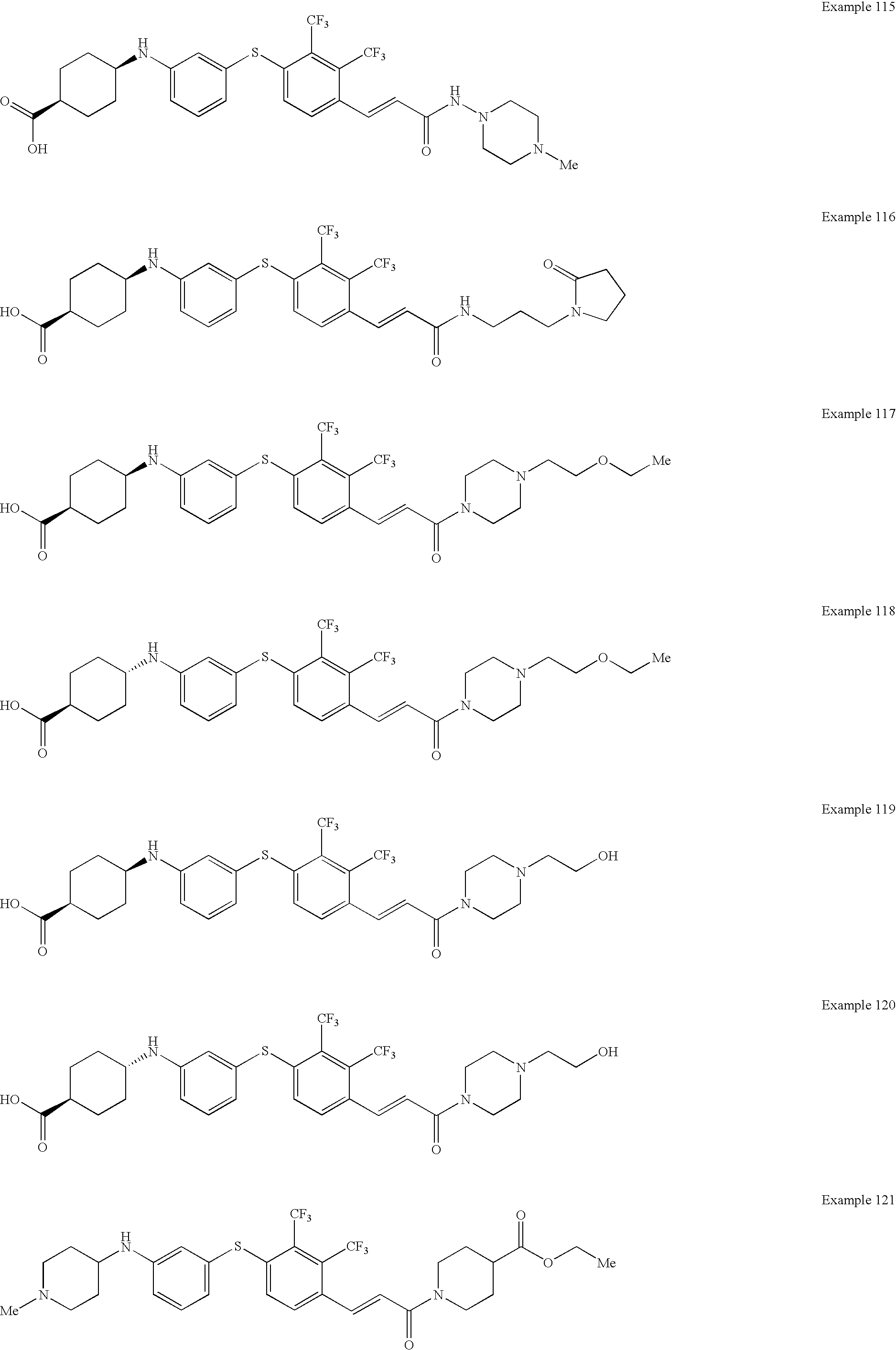

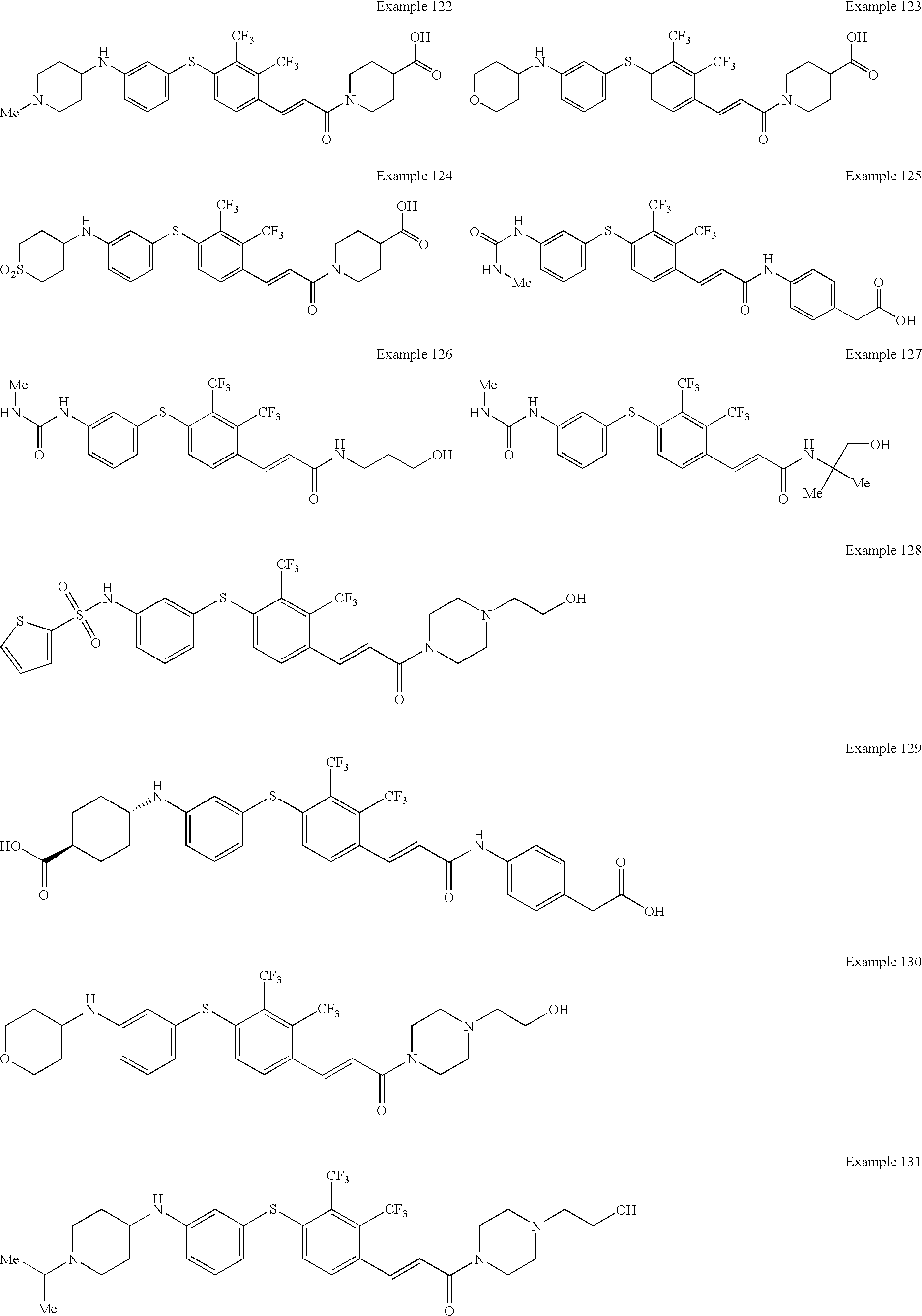

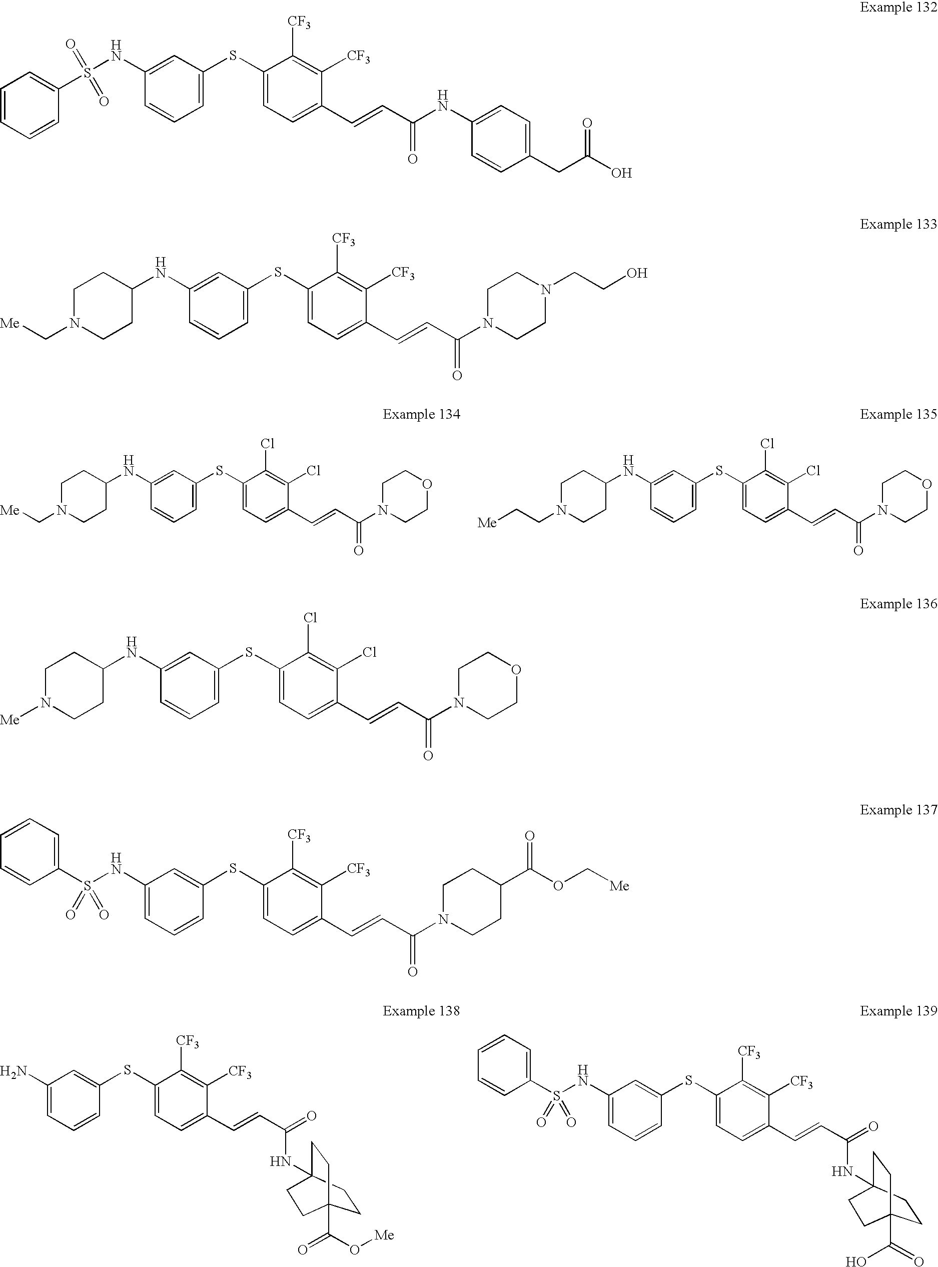

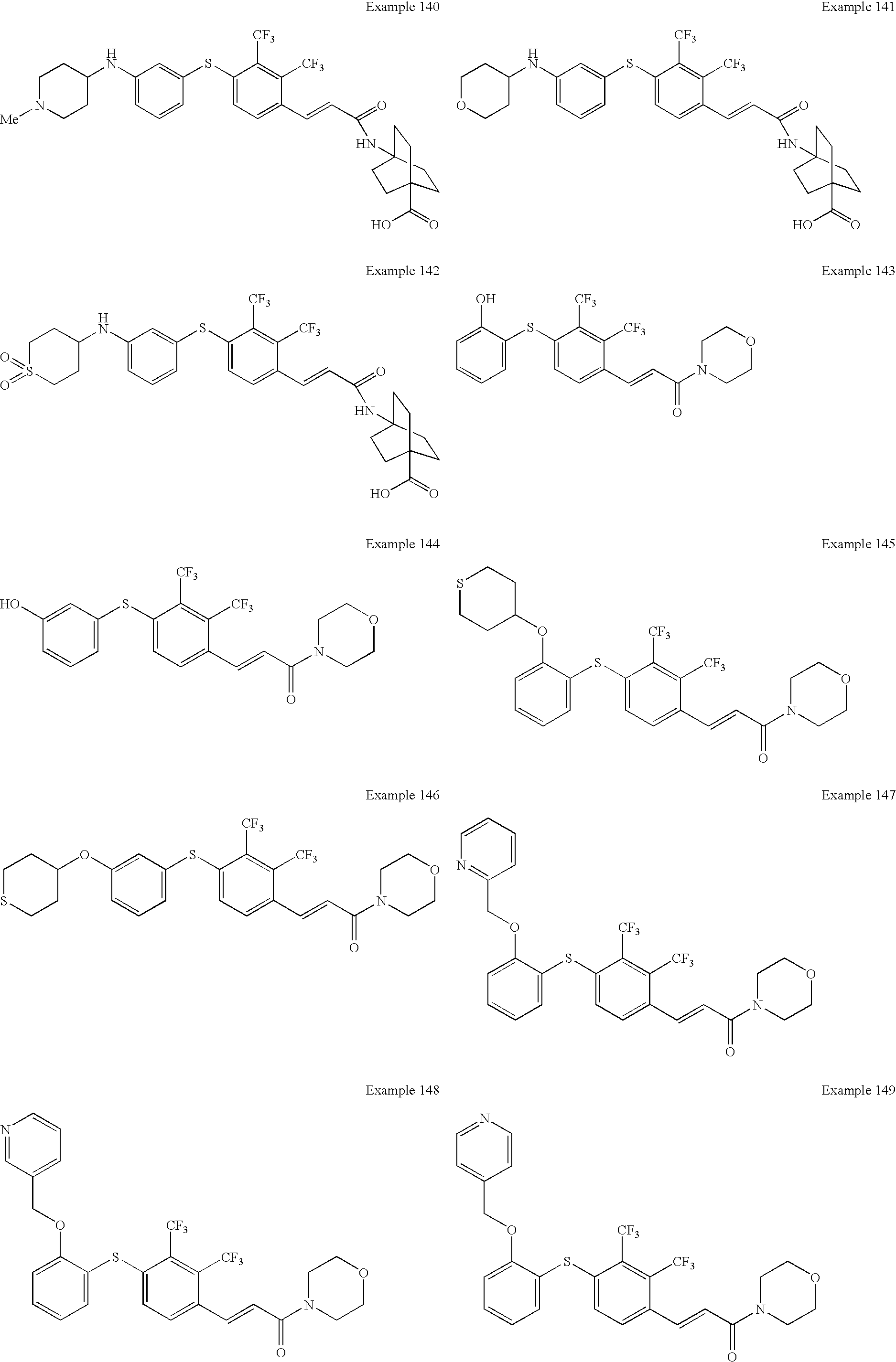

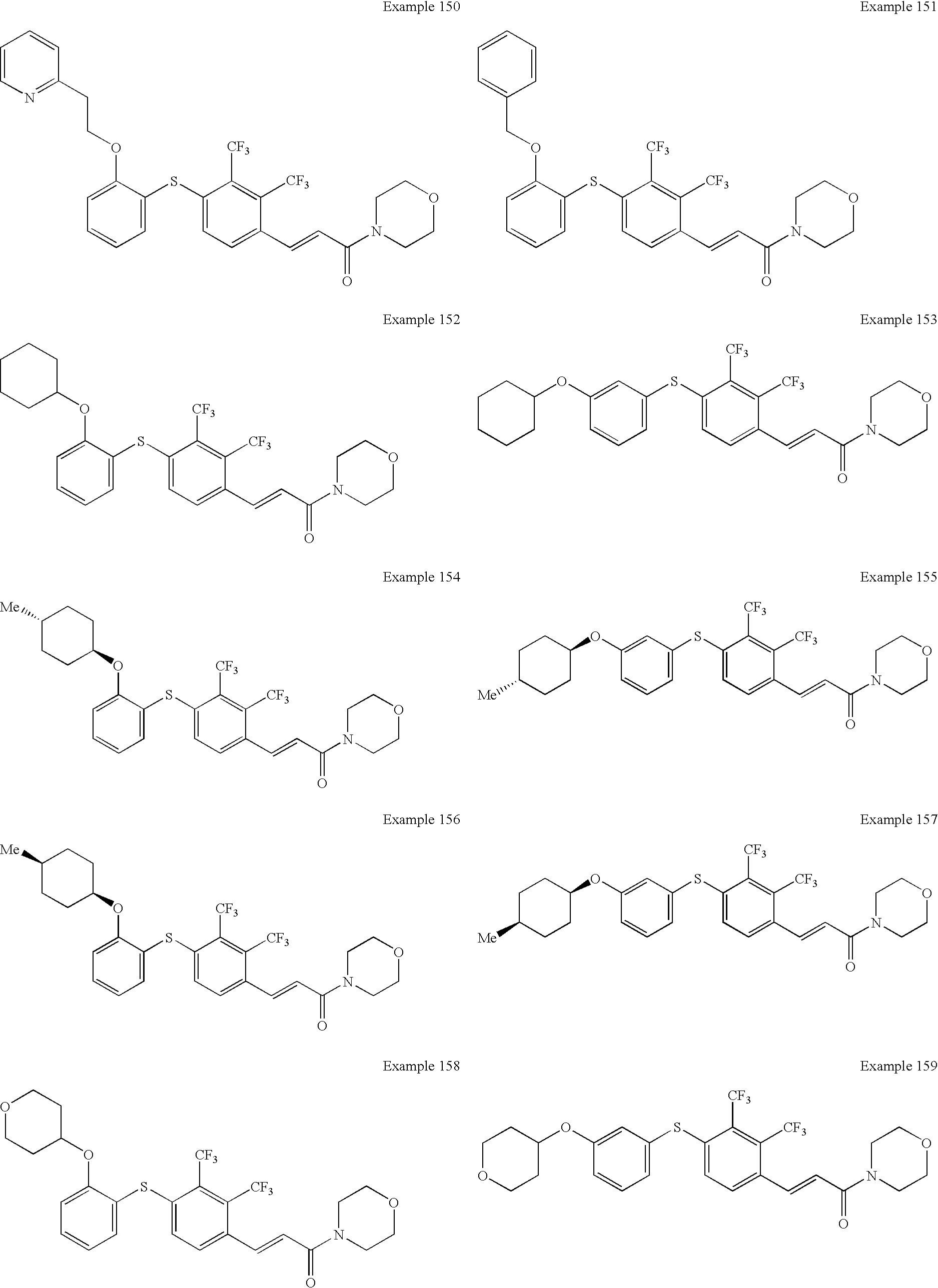

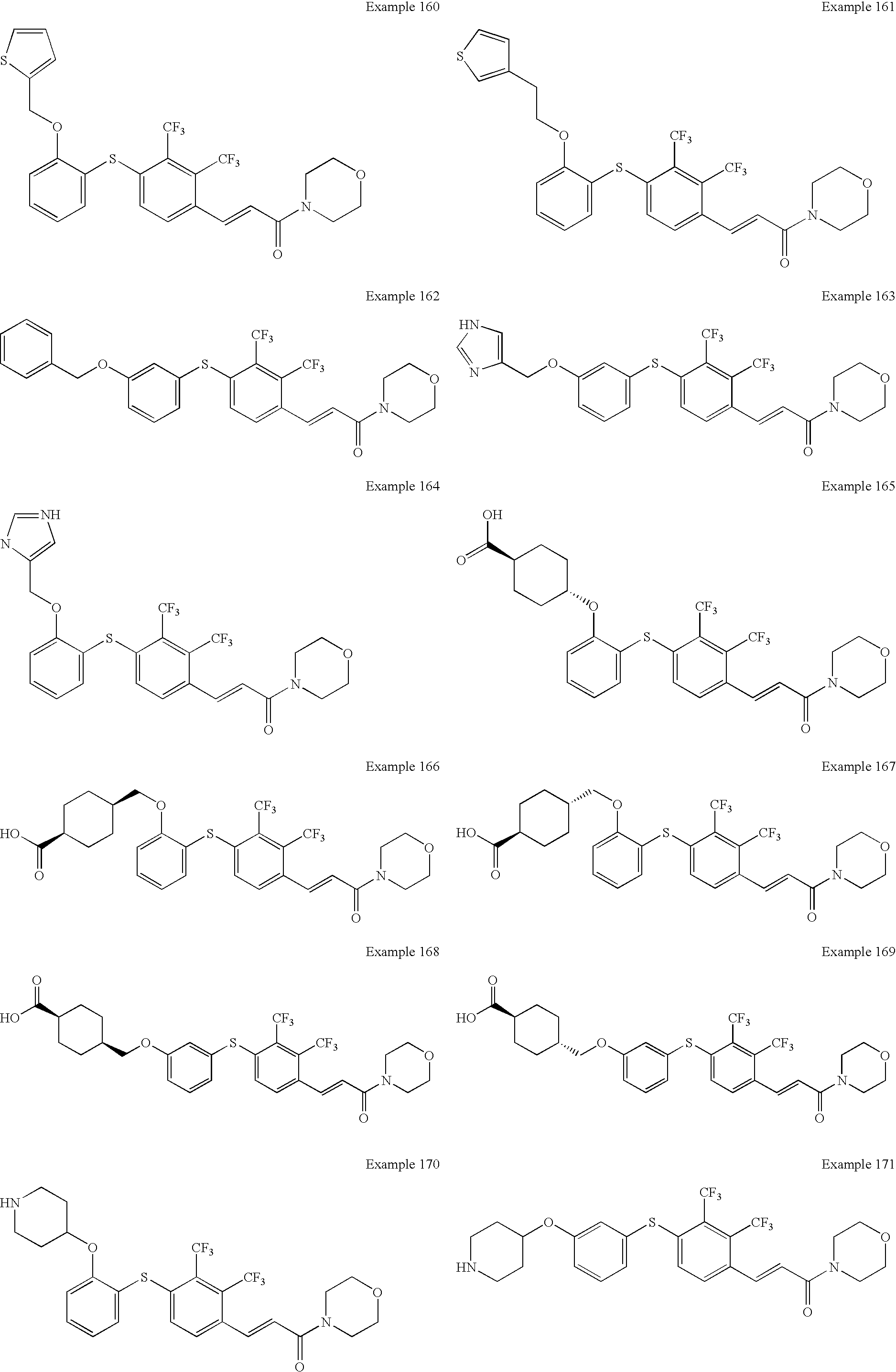

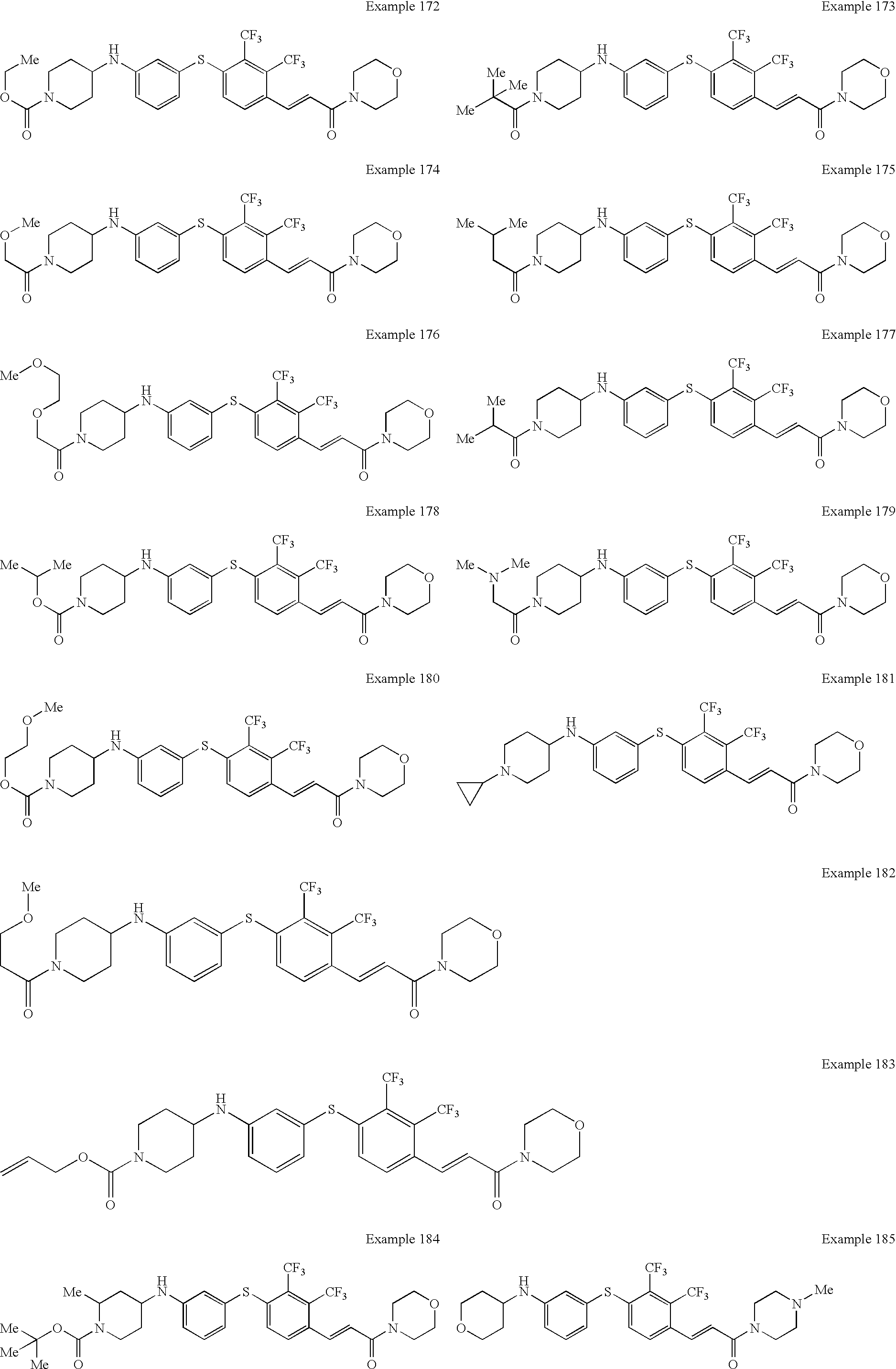

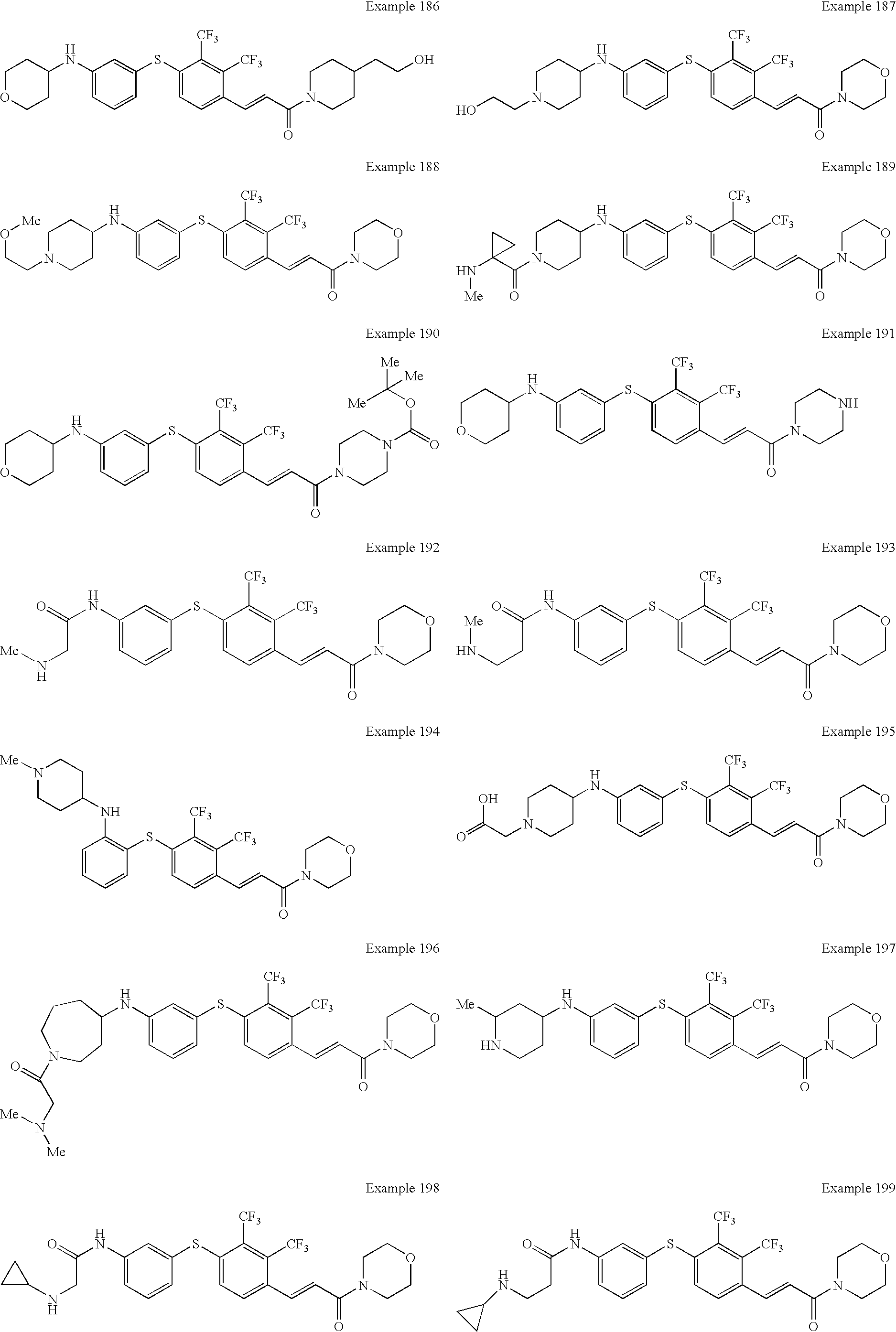

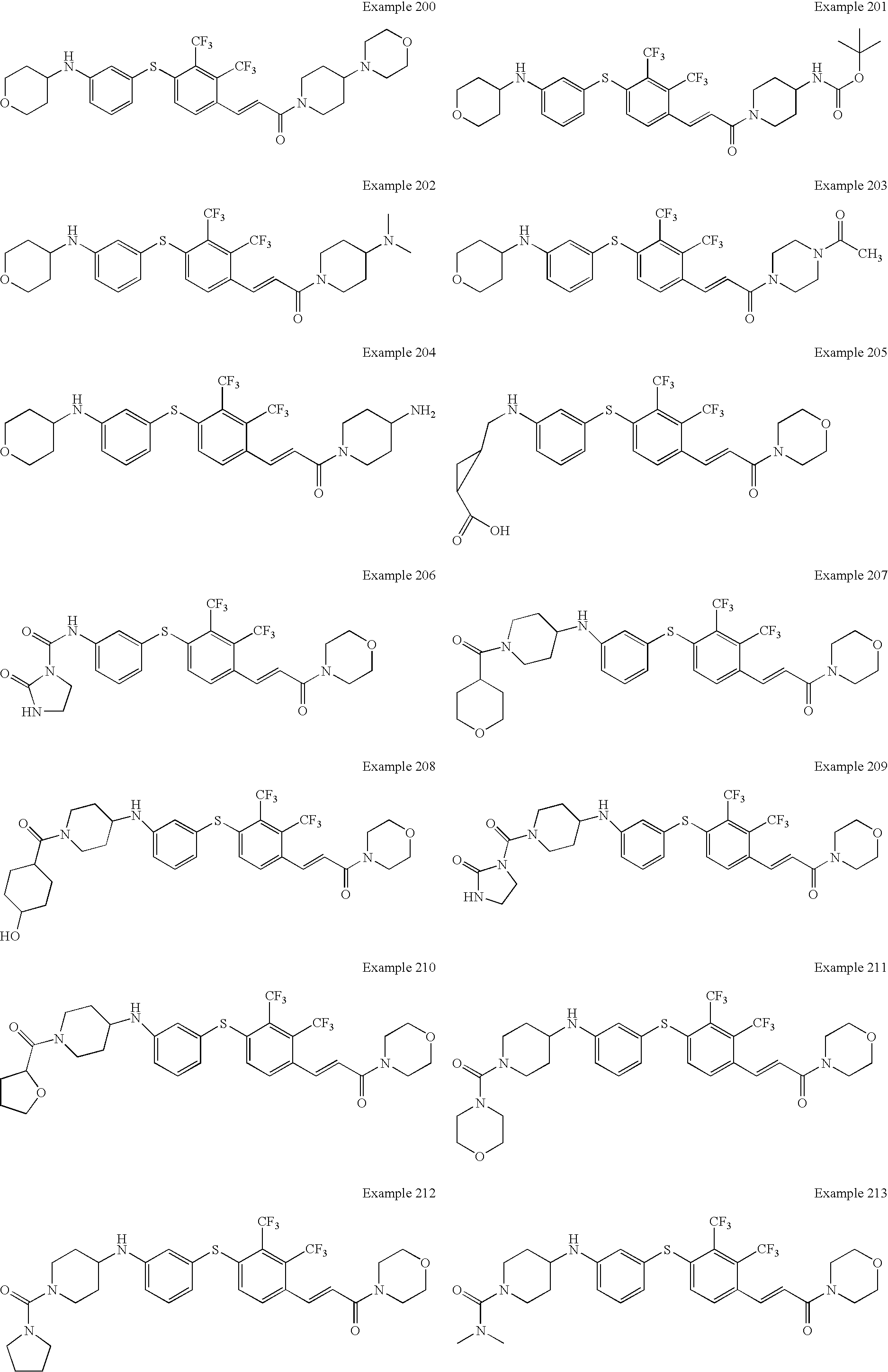

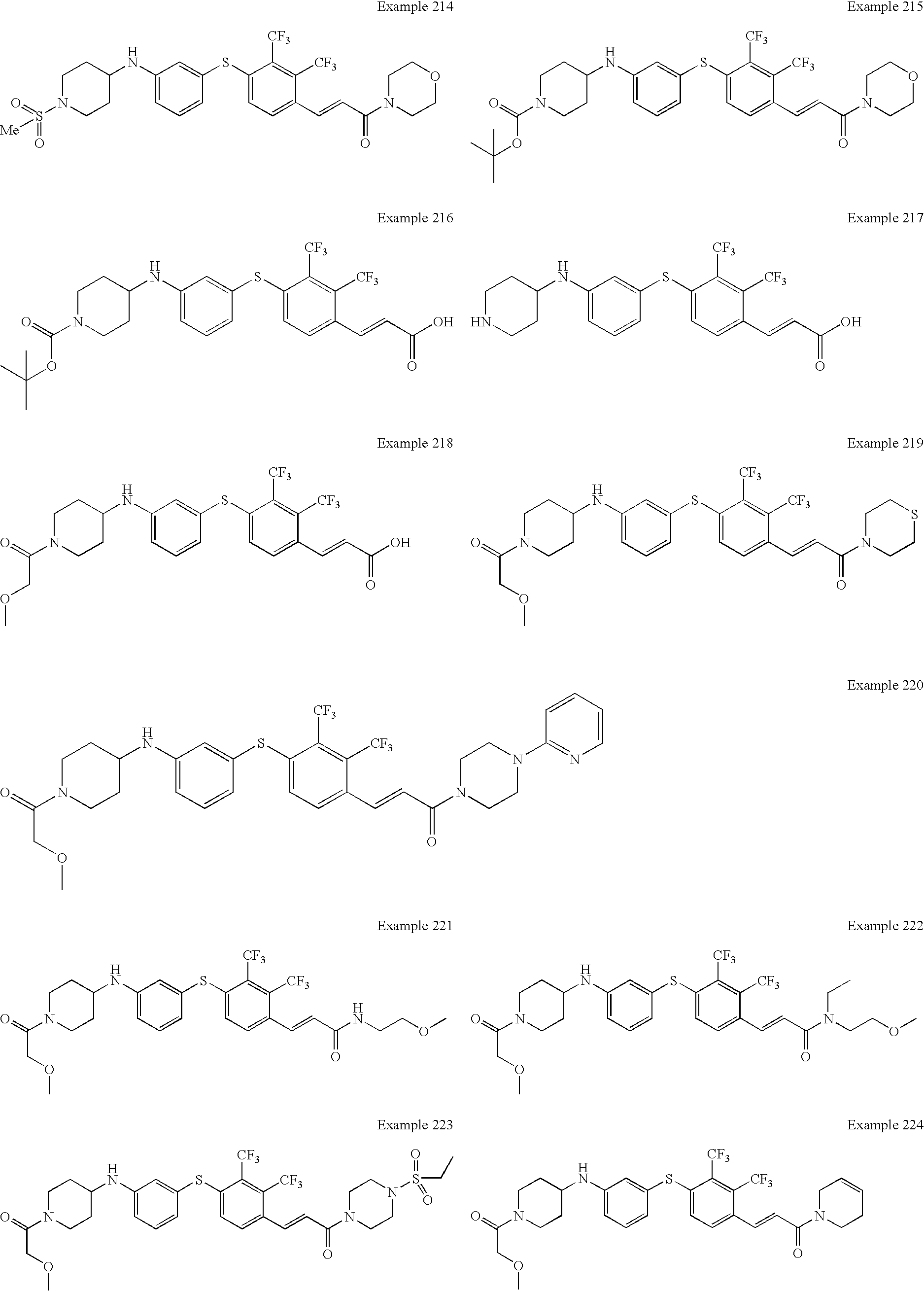

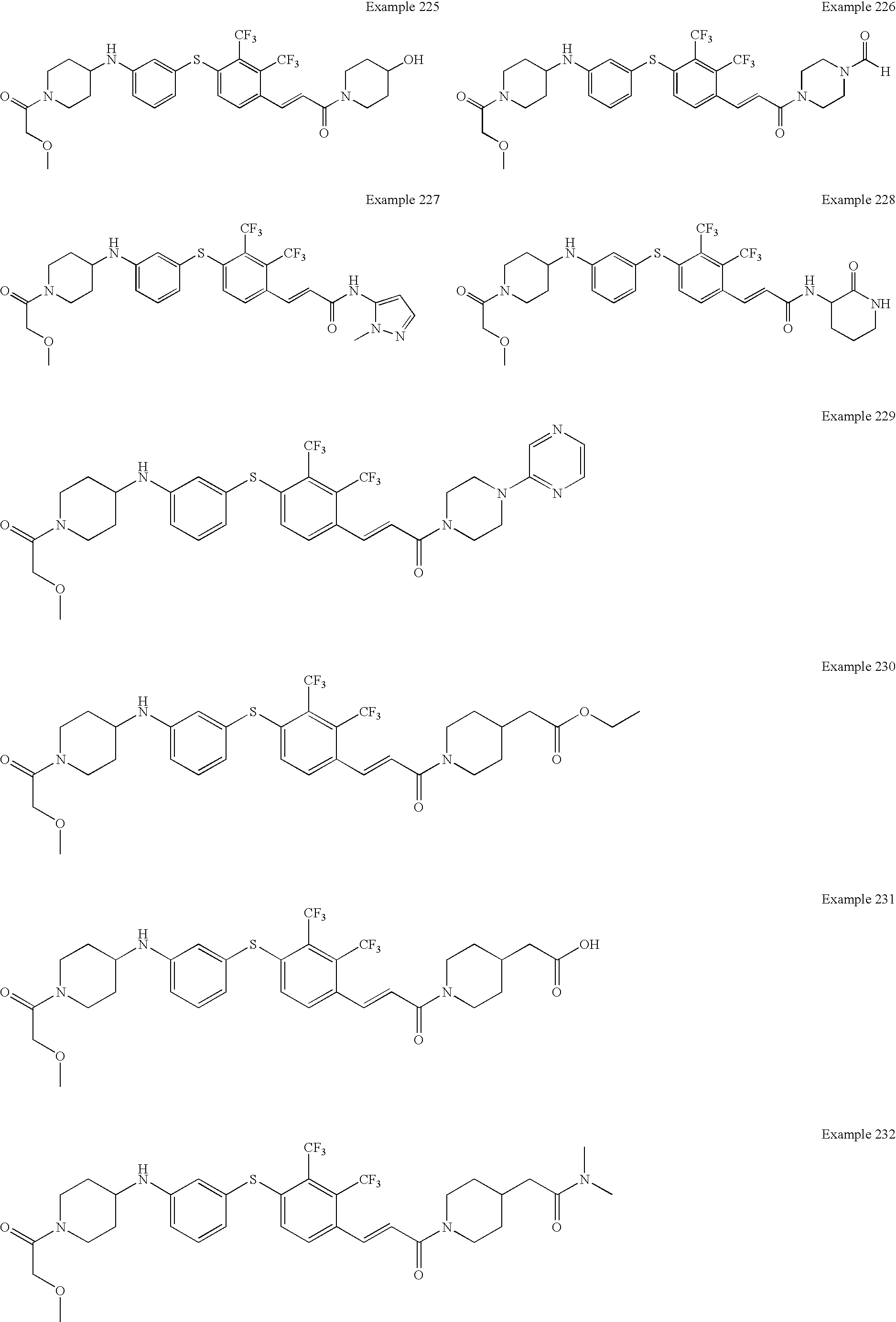

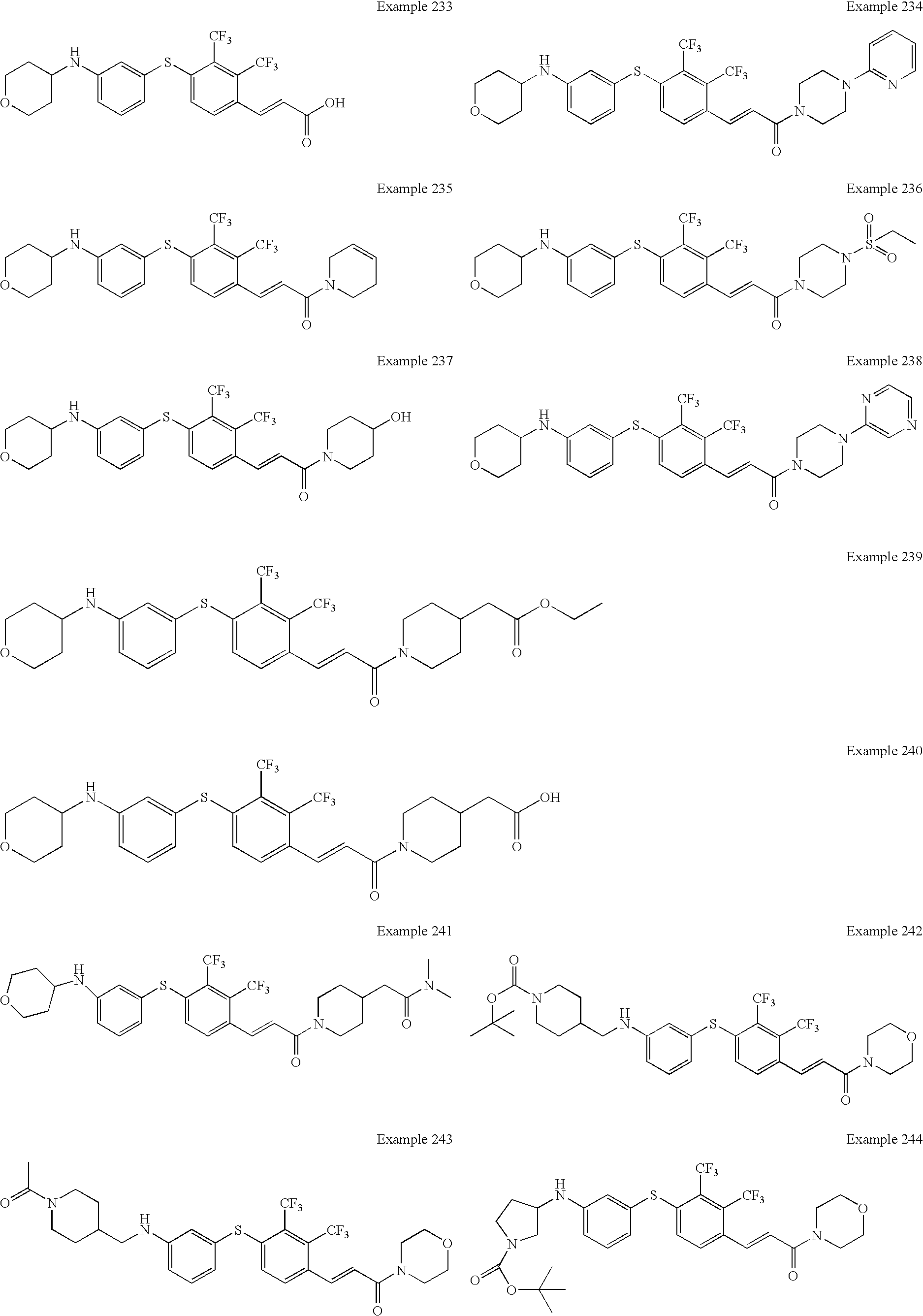

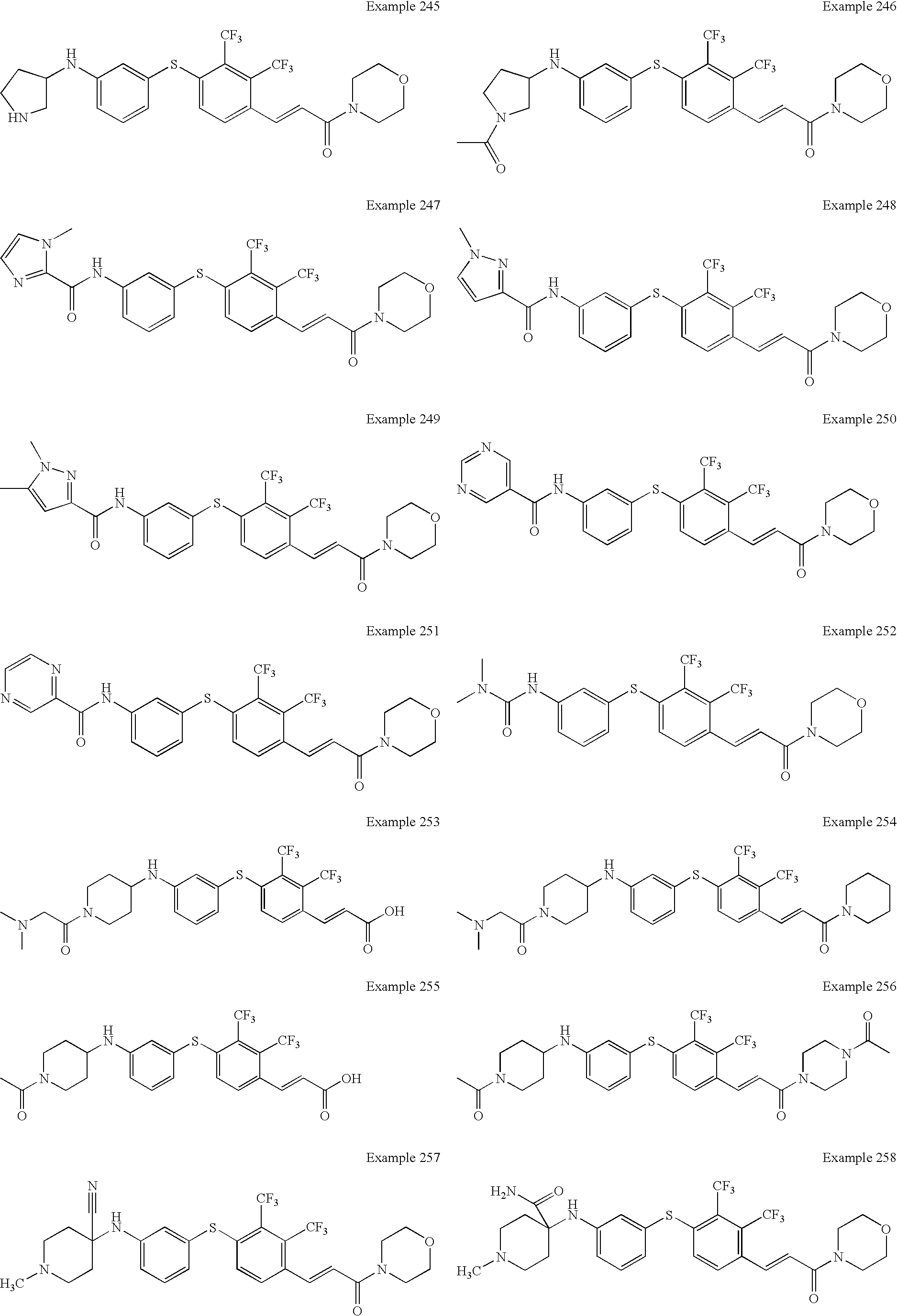

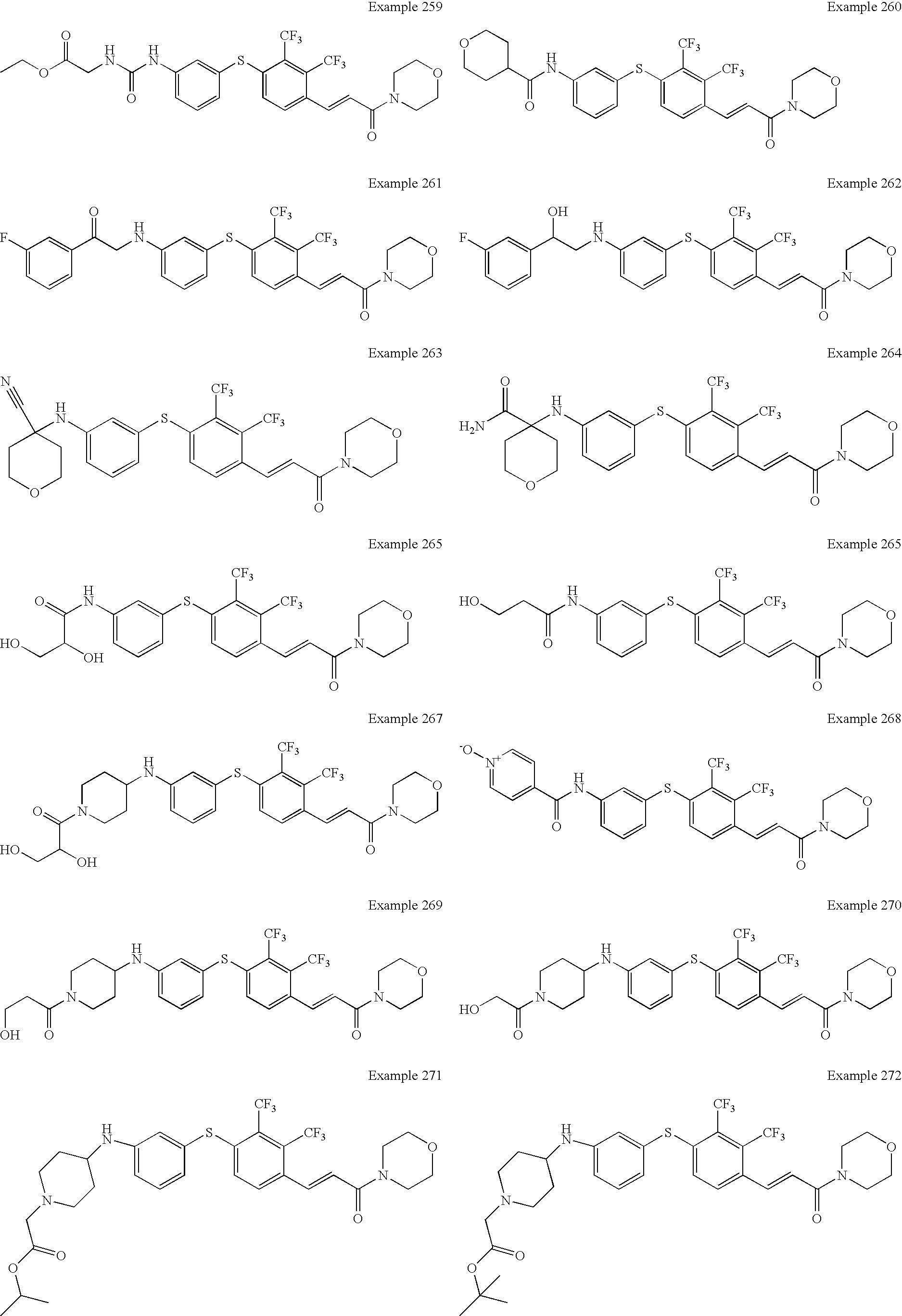

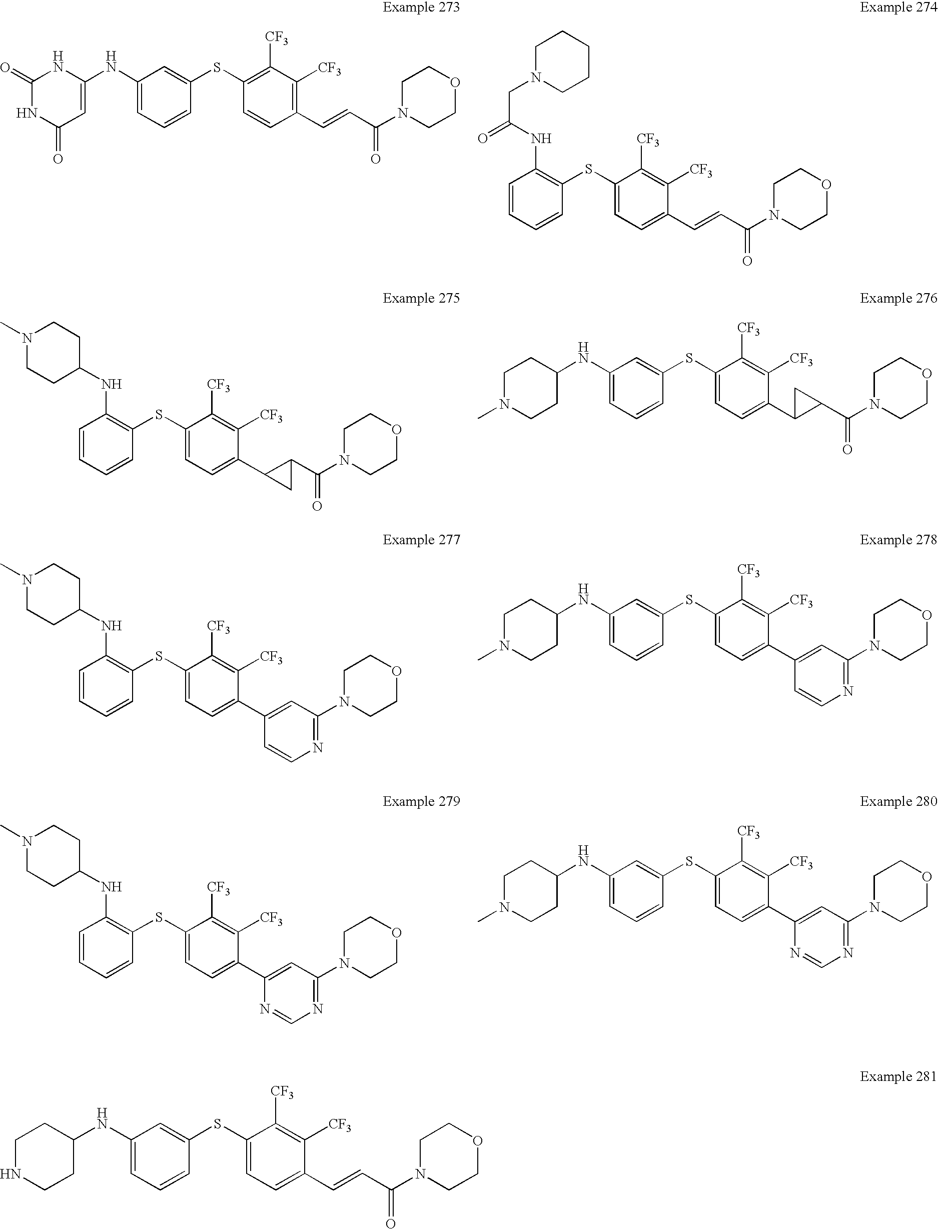

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound of formula I: 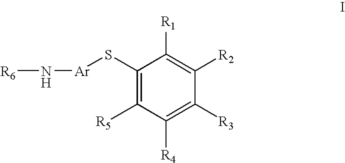
and pharmaceutically-acceptable salts and prodrugs thereof,
wherein R₁, R₂, R₃, R₄, R₅ are each independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and carbonyl-containing groups selected from arylcarbonyl, cycloalkylcarbonyl, and heterocyclylcarbonyl,
wherein R₆ is selected from aldehyde, alkanoyl, alkenyl, alkenoxy, alkoxy, alkynyl, amido, amino, aminothiocarbonyl, aryl, arylcarbonyl, aryloxy, carboxy, cyano, ester, ether, heterocyclyl, heterocyclylcarbonyl, ketone, nitro, perfluoroalkyl, substituted alkyl, substituted carboxyalkyl, substituted cycloalkyl, substituted heterocyclylalkyl, sulfonyl, and sulfonate,
with the proviso that at least one of R₁ and R₃ is selected from:
A. cinnamides selected from cis-cinnamide and trans-cinnamide defined as 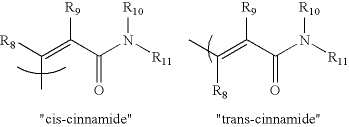
wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, heterocyclyl, hydroxy, ketone, nitro, sulfonate, sulfonyl, thio, and carbonyl-containing groups selected from arylcarbonyl, cycloalkylcarbonyl, and heterocyclylcarbonyl;
B. substituents of formula IV: 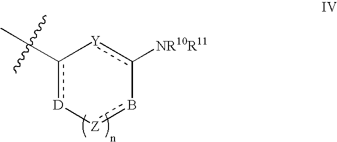
wherein D, B, Y and Z are each independently selected from —CR³¹═, —CR³²R³³—, —C(O)—, —O—, —SO₂—, —S—, —N═, and —NR³⁴—;
n is an integer of zero to three;
R³¹, R³², R³³ and R³⁴ are each independently selected from hydrogen, alkyl, carboxy, hydroxyalkyl, monoalkylaminocarbonylalkyl, dialkylaminocarbonylalkyl and carboxyalkyl; and
C. cyclopropyl derivatives selected from cis-cyclopropanoic acid, trans-cyclopropanoic acid, cis-cyclopropanamide and trans-cyclopropanamide defined as 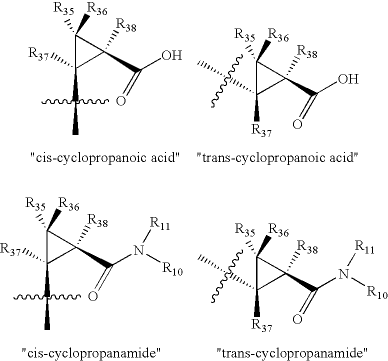
wherein R₃₅, R₃₆, R₃₇, and R₃₈ are each independently selected from hydrogen, alkyl, carboxy, carboxyalkyl, hydroxyalkyl, carboxyalkyl, monoalkylaminocarbonylalkyl, and dialkylaminocarbonylalkyl;
D. substituents of formula VI: 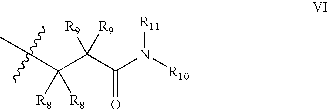
wherein R₈ and R₉ are as defined above; and
E. cinnamic acids of formula VII: 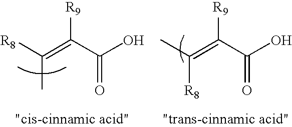
wherein R₈ and R₉ are as defined above;
wherein:
R₁₀ and R₁₁ are each independently selected from hydrogen, alkyl, alkanoyl, alkenyl, alkynyl, alkoxy, amido, aryl, arylalkyl, carboxy, cyano, cycloalkyl, ester, ether, heterocyclyl, hydroxy, ketone, nitro, sulfonyl, thio, and carbonyl-containing groups selected from arylcarbonyl, cycloalkylcarbonyl, and heterocyclylcarbonyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and carbonyl-containing groups selected from arylcarbonyl, cycloalkylcarbonyl, and heterocyclylcarbonyl, and
wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and carbonyl-containing groups selected from arylcarbonyl, cycloalkylcarbonyl, and heterocyclylcarbonyl,
wherein R₁ and R₂, and R₄ and R₅ can be joined to form a 5- to 7-membered cycloalkyl, aryl or heterocyclyl ring when R₃ is selected from cinnamides, substituents of formula IV, substituents of formula VI, substituents of formula VII, and cyclopropyl derivatives as defined above, and R₂ and R₃, R₃ and R₄, and R₄ and R₅ can be joined to form a 5- to 7-membered cycloalkyl, aryl or heterocyclyl ring when R₁ is selected from cinnamides, substituents of formula IV, substituents of formula VI, substituents of formula VII, and cyclopropyl derivatives as defined above,
with the proviso that R₆ is not unsubstituted alkyl, unsubstituted saturated cycloalkyl, unsubstituted carboxyalkyl wherein the alkyl is bonded to the NH group of the parent compound, or unsubstituted heterocyclylalkyl wherein the alkyl is bonded to the NH group of the parent compound.

2. A compound of formula I: 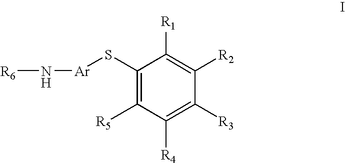
and pharmaceutically-acceptable salts and prodrugs thereof,
wherein R₁, R₂, R₃, R₄, R₅ are each independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,
wherein R₆ is selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,
with the proviso that at least one of R₁ and R₃ is selected from:
A. cinnamides selected from cis-cinnamide and trans-cinnamide defined as 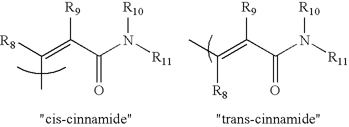
wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, heterocyclyl, hydroxy, ketone, nitro, and carbonyl-containing groups selected from arylcarbonyl, cycloalkylcarbonyl, and heterocyclylcarbonyl;
B. substituents of formula IV: 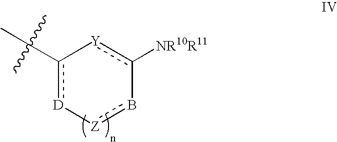
wherein D, B, Y and Z are each independently selected from —CR³¹═, —CR³²R³³—, —C(O)—, —O—, —SO₂—, —S—, —N═, and —NR³⁴—;
n is an integer of zero to three; and
R³¹, R³², R³³ and R³⁴ are each independently selected from hydrogen, alkyl, carboxy, hydroxyalkyl, monoalkylaminocarbonylalkyl, dialkylaminocarbonylalkyl and carboxyalkyl;
C. cyclopropyl derivatives selected from cis-cyclopropanoic acid, trans-cyclopropanoic acid, cis-cyclopropanamide and trans-cyclopropanamide defined as 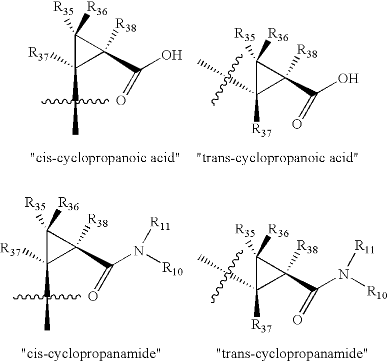
wherein R₃₅, R₃₆, R₃₇, and R₃₈ are each independently selected from hydrogen, alkyl, carboxy, carboxyalkyl, hydroxyalkyl, carboxyalkyl, monoalkylaminocarbonylalkyl, and dialkylaminocarbonylalkyl;
D. substituents of formula VI: 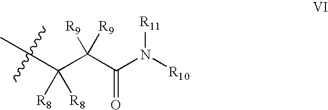
wherein R₈ and R₉ are as defined above; and
E. cinnamic acids of formula VII: 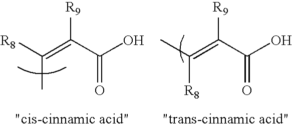
wherein R₈ and R₉ are as defined above;
wherein:
R₁₀ and R₁₁ are each independently selected from hydrogen, alkanoyl, alkyl, alkenyl, alkynyl, alkoxy, amido, aryl, arylalkyl, carboxy, cyano, cycloalkyl, ester, ether, heterocyclyl, hydroxy, ketone, nitro, and carbonyl-containing groups selected from arylcarbonyl, cycloalkylcarbonyl, and heterocyclylcarbonyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl, and
wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl, and
wherein R₁ and R₂, and R₄ and R₅ can be joined to form a 5- to 7-membered cycloalkyl, aryl or heterocyclyl ring when R₃ is selected from cinnamides, substituents of formula IV, substituents of formula VI, substituents of formula VII, and cyclopropyl derivatives as defined above, and R₂ and R₃, R₃ and R₄, and R₄ and R₅ can be joined to form a 5- to 7-membered cycloalkyl, aryl or heterocyclyl ring when R₁ is selected from cinnamides, substituents of formula IV, substituents of formula VI, substituents of formula VII, and cyclopropyl derivatives as defined above,
with the proviso that:
(i) when R₆ is hydrogen, then R₁₀ or R₁₁ is a cycloalkyl; and
(ii) R₆ is not unsubstituted alkyl, unsubstituted saturated cycloalkyl, unsubstituted carboxyalkyl wherein the alkyl is bonded to the NH group of the parent compound, or unsubstituted heterocyclylalkyl wherein the alkyl is bonded to the NH group of the parent compound.

3. The compound according to claim 1, wherein R₆ is selected from 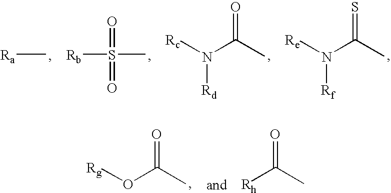
wherein:
Rₐ is selected from alkenyl, alkynyl, aryl, amino, carboxy, cyano, ether, heterocyclyl, ketone, nitro,
substituted alkyl with at least one substituent selected from alkylthio, aldehyde, alkoxy, amido, amino, aminothiocarbonyl, aryl, arylthio, carboxy, cyano, cycloalkyl, ester, ether, halogen, heterocyclyl, hydroxy, ketone, nitro, sulfonate, sulfonyl, and thiol, and
substituted cycloalkyl, with at least one substituent selected from alkyl, alkylthio, aldehyde, alkanoyl, alkoxy, amido, amino, aminothiocarbonyl, aryl, arylthio, carboxy, carboxyalkyl, cyano, cycloalkyl, ester, ether, halogen, heterocyclyl, hydroxy, ketone, nitro, sulfonate, sulfonyl, and thiol;
R_{b} is selected from alkyl, alkanoyl, alkenyl, alkynyl, alkoxy, amino, amido, aryl, cycloalkyl, carboxyalkyl, cyano, ester, ether, halogen, heterocyclyl, hydroxy, and ketone;
R_{c}, R_{d}, Rₑ, and R_{f} are each independently selected from hydrogen, alkanoyl, alkyl, alkenyl, alkynyl, alkoxy, amino, amido, aryl, carboxy, cycloalkyl, ester, ether, ketone, nitro, and heterocyclyl, or R_{c} and R^{d}, or Rₑ and R_{f} may be joined together to form a substituted or unsubstituted 3- to 12-membered cycloalkyl ring, or a substituted or unsubstituted 3- to 12-membered heterocyclyl ring, which comprises one or more atoms selected from N, O, and S,
wherein the substituted cycloalkyl or heterocyclyl ring comprises at least one substituent selected from alkyl, alkylthio, alkanoyl, alkenyl, alkynyl, aldehyde, alkoxy, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, nitro, oxo, sulfonate, sulfonyl, and thiol;
R_{g} is selected from hydrogen, alkyl, alkanoyl, aldehyde, alkenyl, alkoxy, alkynyl, amido, amino, aryl, arylcarbonyl, carboxy, cycloalkyl, cycloalkylcarbonyl, ester, ether, heterocyclyl, heterocyclylcarbonyl, and ketone; and
Rₕ is selected from hydrogen, alkyl, alkylthio, alkenyl, alkynyl, alkanoyl, aldehyde, alkoxy, aryl, arylcarbonyl, arylthio, amido, carboxy, cycloalkyl, cycloalkylcarbonyl, ester, ether, halogen, heterocyclyl, heterocyclylcarbonyl, ketone, nitro, sulfonate, sulfonyl, and thiol.

4. A compound of formula III: 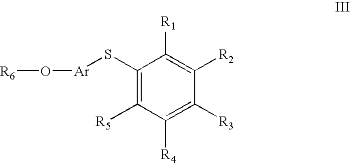
and pharmaceutically-acceptable salts and prodrugs thereof,
wherein R₁, R₂, R₃, R₄, and R₅ are each independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl;
wherein R₆ is selected from alkenyl, alkenoxy, alkynyl, aldehyde, alkanoyl, alkoxy, amido, amino, aryl, aryloxy, a carbonyl-containing group selected from arylcarbonyl, cycloalkylcarbonyl, and heterocyclylcarbonyl; carboxy, cyano, ether, ester, halogen, heterocyclyl, hydroxy, ketone, nitro, perfluoroalkyl, substituted alkyl, carboxyalkyl, substituted cycloalkyl, heterocyclylalkyl, sulfonyl, sulfonate, and thio groups selected from alkylthio, arylthio, and thiol;
with the proviso that at least one of R₁ and R₃ is selected from:
A. cinnamides selected from cis-cinnamide and trans-cinnamide defined as 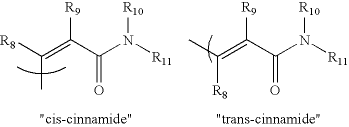
wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, heterocyclyl, hydroxy, ketone, nitro, and carbonyl-containing groups selected from arylcarbonyl, cycloalkylcarbonyl, and heterocyclylcarbonyl;
B. substituents of formula IV: 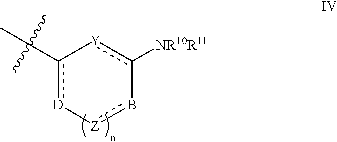
wherein D, B, Y and Z are each independently selected from the —CR³¹═, —CR³²R³³—, —C(O)—, —O—, —SO₂—, —S—, —N═, and —NR³⁴—;
n is an integer of zero to three; and
R³¹, R³², R³³ and R³⁴ are each independently selected from hydrogen, alkyl, carboxy, hydroxyalkyl, monoalkylaminocarbonylalkyl, dialkylaminocarbonylalkyl and carboxyalkyl;
C. cyclopropyl derivatives selected from cis-cyclopropanoic acid, trans-cyclopropanoic acid, cis-cyclopropanamide and trans-cyclopropanamide defined as 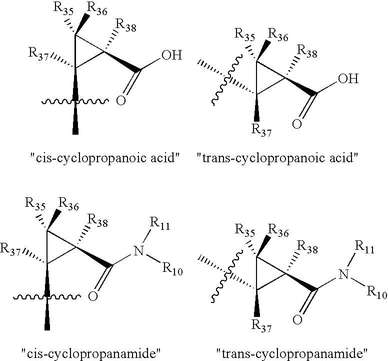
wherein R₃₅ and R₃₆ are each independently selected from hydrogen, alkyl, carboxy, hydroxyalkyl, and carboxyalkyl, and
wherein R₃₇ and R₃₈ are each independently selected from hydrogen, alkyl, carboxyalkyl, monoalkylaminocarbonylalkyl, and dialkylaminocarbonylalkyl;
D. substituents of formula VI: 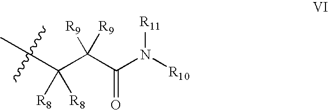
wherein R₈ and R₉ are as defined above; and
E. cinnamic acids of formula VII: 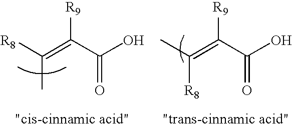
wherein R₈ and R₉ are as defined above;
wherein:
R₁₀ and R₁₁ are each independently selected from hydrogen, alkanoyl, alkyl, alkenyl, alkynyl, alkoxy, amido aryl, arylalkyl, carboxy, cyano, cycloalkyl, ester, ether, heterocyclyl, hydroxy, ketone, nitro, and carbonyl-containing groups selected from arylcarbonyl, cycloalkylcarbonyl, and heterocyclylcarbonyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl, and
wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl, and
wherein R₁ and R₂, and R₄ and R₅ can be joined to form a 5- to 7-membered cycloalkyl, aryl or heterocyclyl ring when R₃ is selected from cinnamides, substituents of formula IV, substituents of formula VI, substituents of formula VII, and cyclopropyl derivatives as defined above, and R₂ and R₃, R₃ and R₄, and R₄ and R₅ can be joined to form a 5- to 7-membered cycloalkyl, aryl or heterocyclyl ring when R₁ is selected from cinnamides, substituents of formula IV, substituents of formula VI, substituents of formula VII, and cyclopropyl derivatives as defined above,
with the proviso that when R₆ is substituted cycloalkyl, the substituent is not a carboxy group.

5. The compound according to claim 1, wherein R₆ is selected from alkylthio, alkenyl, alkenoxy, alkynyl, aldehyde, alkanoyl, alkoxy, amido, amino, aryl, arylthio, arylcarbonyl, aryloxy, carboxy, cycloalkylcarbonyl, ether, ester, heterocyclyl, heterocyclylcarbonyl, ketone, nitro, perfluoroalkyl, substituted alkyl, substituted carboxyalkyl, substituted cycloalkyl, substituted heterocyclylalkyl, sulfonyl, sulfonate, and thiol.

6. The compound according to claim 1, wherein R₆ is selected from alkanoyl, alkanoylalkyl, amino, amido, aryl, arylalkyl, arylcarbonyl, carboxycycloalkylalkyl, cycloalkylcarbonyl, heterocyclyl, heterocyclylalkyl, heterocyclylcarbonyl, and sulfonyl.

7. The compound according to claim 1, wherein R₆ is an alkanoyl comprising an alkyl group bonded to a carbonyl group, wherein the alkyl group is unsubstituted or substituted with at least one group selected from alkylthio, aldehyde, alkoxy, amido, amino, aminothiocarbonyl, aryl, arylthio, carboxy, cyano, cycloalkyl, ester, ether, halogen, heterocyclyl, hydroxy, ketone, nitro, sulfonate, sulfonyl, and thiol.

8. The compound according to claim 1, wherein R₆ is an alkanoyl comprising an alkyl group substituted with at least one group selected from alkoxy, alkyl, amino, and heterocyclyl.

9. The compound according to claim 7, wherein R₆ is an alkanoyl comprising an alkyl group substituted with at least one group selected from amino and hydroxy.

10. The compound according to claim 1, wherein R₆ is a cycloalkyl substituted with at least one group selected from alkyl, alkylthio, aldehyde, alkanoyl, alkoxy, amido, amino, aminothiocarbonyl, aryl, arylthio, carboxy, carboxyalkyl, cyano, cycloalkyl, ester, ether, halogen, heterocyclyl, hydroxy, ketone, nitro, sulfonate, sulfonyl, and thiol.

11. The compound according to claim 10, wherein R₆ is a cycloalkyl substituted with at least one group selected from alkyl, carboxy, and carboxyalkyl.

12. The compound according to claim 1, wherein R₆ is a heterocyclyl that is unsubstituted or substituted with at least one group selected from alkyl, alkylthio, alkanoyl, alkenyl, alkynyl, aldehyde, alkoxy, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, nitro, oxo, sulfonate, sulfonyl, and thiol.

13. The compound according to claim 12, wherein R₆ is a heterocyclyl substituted with at least one group selected from alkyl, alkanoyl, amido, arylcarbonyl, cyano, cycloalkyl, cycloalkylcarbonyl, ester, heterocyclylcarbonyl, sulfonyl, and oxo.

14. The compound according to claim 13, wherein R₆ is a heterocyclyl substituted with an alkyl that is substituted with at least one group selected from aryl, alkoxy, alkoxycarbonyl, carboxy, and hydroxy.

15. The compound according to claim 13, wherein R₆ is a heterocyclyl substituted with at least one group selected from alkanoyl and ester, wherein the carbonyl of the alkanoyl and ester is bonded to a substituent selected from alkenoxy, alkoxyalkoxy, alkoxyalkoxyalkyl, alkoxyalkyl, aminoalkyl, and hydroxyalkyl.

16. The compound according to claim 1, wherein R₆ is an alkyl substituted with at least one group selected from alkylthio, aldehyde, alkoxy, amido, amino, aminothiocarbonyl, aryl, arylthio, carboxy, cyano, cycloalkyl, ester, ether, halogen, heterocyclyl, hydroxy, ketone, nitro, sulfonate, sulfonyl, and thiol.

17. The compound according to claim 1, wherein R₆ is an alkyl substituted with at least one group selected from amido, amino, aryl, arylcarbonyl, carboxycycloalkyl, cycloalkyl, and heterocyclyl.

18. The compound according to claim 17, wherein R₆ is an alkyl substituted with a heterocyclyl that is substituted with at least one group selected from alkyl, alkanoyl, and alkoxycarbonyl.

19. The compound according to claim 17, wherein R₆ is an alkyl substituted with an aryl that is substituted with a hydroxy group.

20. The compound according to claim 1, wherein R₆ is an amido substituted with at least one group selected from hydrogen, alkylthio, alkanoyl, alkenyl, alkoxy, alkyl, alkynyl, amido, amino, aryl, arylthio, carboxy, cycloalkyl, ester, ether, halogen, heterocyclyl, hydroxy, ketone, nitro, sulfonate, sulfonyl, and thiol.

21. The compound according to claim 20, wherein R₆ is an amido substituted with at least one group selected from alkyl, alkanoyl, aryl, arylalkyl, carboxyalkyl, cycloalkyl, heterocyclylalkyl, and hydroxyalkyl.

22. The compound according to claim 1, wherein R₆ is a thioamido.

23. The compound according to claim 21, wherein R₆ is an amido substituted with an alkanoyl that is substituted with an alkoxy group.

24. The compound according to claim 1, wherein R₆ is selected from alkanoyl, alkoxycarbonyl, alkoxyalkylcarbonyl, arylalkoxycarbonyl, aryloxycarbonyl, cycloalkylcarbonyl, ester, heterocyclylcarbonyl, heterocyclylalkylcarbonyl, hydroxyalkylcarbonyl, and thiocarbonyl.

25. The compound according to claim 1, wherein R₆ is a sulfonyl substituted with at least group selected from alkyl, amino, aryl, arylalkyl, haloalkyl, heterocyclyl, heterocyclylalkyl, and sulfonylalkyl.

26. A compound of formula V: 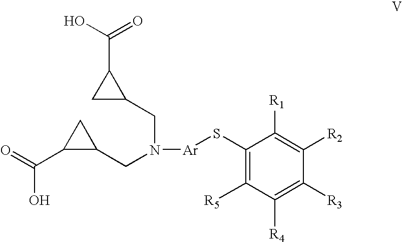
and pharmaceutically-acceptable salts and prodrugs thereof,
wherein R₁, R₂, R₃, R₄, and R₅ are independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,
with the proviso that at least one of R₁ and R₃ is selected from
A. cinnamides selected from cis-cinnamide and trans-cinnamide defined as 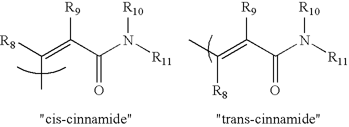
wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, heterocyclyl, hydroxy, ketone, nitro, sulfonate, sulfonyl, thio, and carbonyl-containing groups selected from arylcarbonyl, cycloalkylcarbonyl, and heterocyclylcarbonyl;
B. substituents of formula IV: 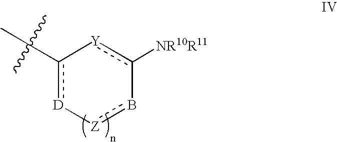
wherein D, B, Y and Z are each independently selected from —CR³¹═, —CR³²R³³—, —C(O)—, —O—, —SO₂—, —S—, —N═, and —NR³⁴—;
n is an integer of zero to three; and
R³¹, R³², R³³ and R³⁴ are each independently selected from hydrogen, alkyl, carboxy, hydroxyalkyl, monoalkylaminocarbonylalkyl, dialkylaminocarbonylalkyl and carboxyalkyl;
C. cyclopropyl derivatives selected from cis-cyclopropanoic acid, trans-cyclopropanoic acid, cis-cyclopropanamide and trans-cyclopropanamide defined as 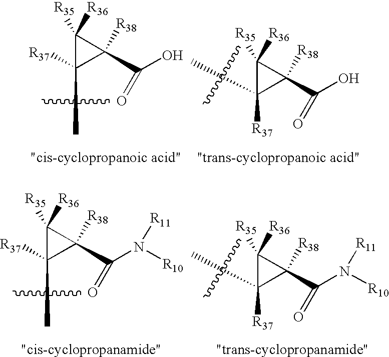
wherein R₃₅ and R₃₆ are each independently selected from hydrogen, alkyl, carboxy, hydroxyalkyl, and carboxyalkyl, and
wherein R₃₇ and R₃₈ are each independently selected from hydrogen, alkyl, carboxyalkyl, monoalkylaminocarbonylalkyl, and dialkylaminocarbonylalkyl;
D. substituents of formula VI: 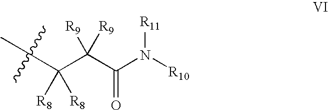
wherein R₈ and R₉ are as defined above; and
E. cinnamic acids of formula VII: 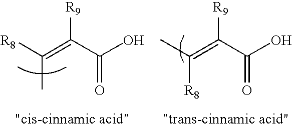
wherein R₈ and R₉ are as defined above;
wherein:
R₁₀ and R₁₁ are each independently selected from hydrogen, alkyl, alkanoyl, alkenyl, alkynyl, alkoxy, amido, aryl, arylalkyl, carboxy, cyano, cycloalkyl, ester, ether, heterocyclyl, hydroxy, ketone, nitro, sulfonyl, thio groups selected from alkylthio, arylthio, and thiol, and carbonyl-containing groups selected from arylcarbonyl, cycloalkylcarbonyl, and heterocyclylcarbonyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl, and
wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,
wherein R₁ and R₂, and R₄ and R₅ can be joined to form a 5- to 7-membered cycloalkyl, aryl or heterocyclyl ring when R₃ is selected from cinnamides, substituents of formula IV, substituents of formula VI, substituents of formula VII, and cyclopropyl derivatives as defined above, and R₂ and R₃, R₃ and R₄, and R₄ and R₅ can be joined to form a 5- to 7-membered cycloalkyl, aryl or heterocyclyl ring when R₁ is selected from cinnamides, substituents of formula IV, substituents of formula VI, substituents of formula VII, and cyclopropyl derivatives as defined above.

27. A compound of formula I: 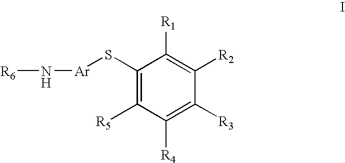
and pharmaceutically-acceptable salts thereof,
wherein R₁, R₂, R₃, R₄, R₅ are each independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,
with the proviso that at least one of R₁ and R₃ is cis-cinnamide or trans-cinnamide is selected from:
A. cinnamides selected from cis-cinnamide and trans-cinnamide defined as 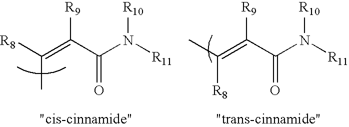
wherein R₆ is selected from alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl,
wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, heterocyclyl, hydroxy, ketone, nitro, sulfonate, sulfonyl, thio groups selected from alkylthio, arylthio, and thiol, and carbonyl-containing groups selected from arylcarbonyl, cycloalkylcarbonyl, and heterocyclylcarbonyl;
B. substituents of formula IV: 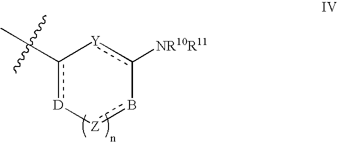
wherein D, B, Y and Z are each independently selected from the group consisting of —CR³¹═, —CR³²R³³—, —C(O)—, —O—, —SO₂—, —S—, —N═, and —NR³—;
n is an integer of zero to three; and
R³¹, R³², R³³ and R³⁴ are each independently selected from the group consisting of hydrogen, alkyl, carboxy, hydroxyalkyl, monoalkylaminocarbonylalkyl, dialkylaminocarbonylalkyl and carboxyalkyl;
C. cyclopropyl derivatives selected from cis-cyclopropanoic acid, trans-cyclopropanoic acid, cis-cyclopropanamide and trans-cyclopropanamide defined as 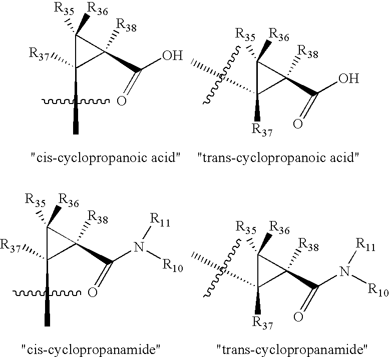
wherein R₃₅, R₃₆, R₃₇, and R₃₈ are each independently selected from hydrogen, alkyl, carboxy, carboxyalkyl, hydroxyalkyl, carboxyalkyl, monoalkylaminocarbonylalkyl, and dialkylaminocarbonylalkyl;
D. substituents of formula VI: 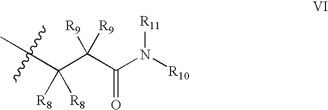
wherein R₈ and R₉ are as defined above; and
E. cinnamic acids of formula VII: 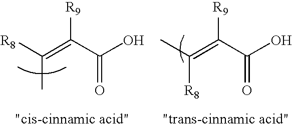
wherein R₈ and R₉ are as defined above;
wherein:
R₁₀ and R₁₁ are each independently selected from hydrogen, alkyl, alkanoyl, alkenyl, alkynyl, alkoxy, amido, aryl, arylalkyl, carboxy, cyano, cycloalkyl, ester, ether, heterocyclyl, hydroxy, ketone, nitro, sulfonyl, thio groups selected from alkylthio, arylthio, and thiol, and carbonyl-containing groups selected from arylcarbonyl, cycloalkylcarbonyl, and heterocyclylcarbonyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl, and
wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio groups selected from alkylthio, arylthio, and thiol, and carbonyl-containing groups selected from arylcarbonyl, cycloalkylcarbonyl, and heterocyclylcarbonyl.

28. A compound of formula I: 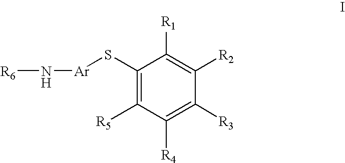
and pharmaceutically-acceptable salts thereof,
wherein R₁, R₂, R₃, R₄, R₅ are each independently selected from hydrogen, alkyl, amino, haloalkyl, and halogen,
wherein R₆ is selected from amido, ester, heterocyclyl, sulfonyl, sulfonate, substituted alkyl, substituted cycloalkyl; and carbonyl-containing groups selected from aminoalkylcarbonyl, arylcarbonyl, cycloalkylcarbonyl, heterocyclylcarbonyl, heterocyclylalkylcarbonyl, and hydroxyalkylcarbonyl,
with the proviso that at least one of R₁ or R₃ is cis-cinnamide or trans-cinnamide is selected from:
A. cinnamides selected from cis-cinnamide or trans-cinnamide defined as 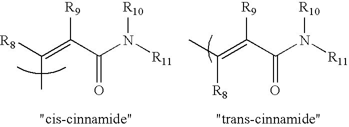
wherein R₈ and R₉ are each hydrogen;
B. substituents of formula IV: 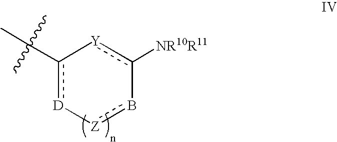
wherein D, B, Y and Z are each independently selected from the group consisting of —CH═ and —N═, and
n is one;
C. cyclopropyl derivatives selected from cis-cyclopropanoic acid, trans-cyclopropanoic acid, cis-cyclopropanamide and trans-cyclopropanamide defined as 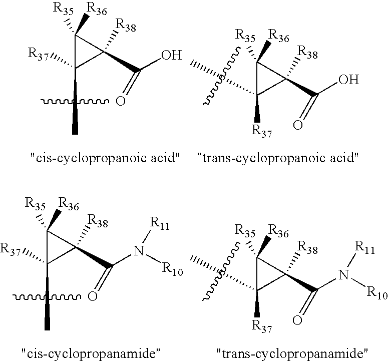
wherein R₃₅, R₃₆, R₃₇, and R₃₈ are each hydrogen; and
D. cinnamic acids of formula VII: 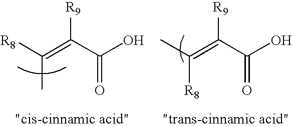
wherein R₈ and R₉ are as defined above;
wherein:
R₁₀ and R₁₁ are each independently selected from hydrogen, alkyl, aryl, arylalkyl, cycloalkyl, ester, ether, and heterocyclyl, or
R₁₀ and R₁₁ are taken together with N to form a heterocyclyl group bonded to at least one substituent independently selected from hydrogen, alkyl, aldehyde, alkanoyl, amido, amino, carboxy, ether, ester, heterocyclyl, hydroxy, ketone, and sulfonyl, and
wherein Ar is phenyl,
with the proviso that R₆ is not unsubstituted carboxyalkyl wherein the alkyl is bonded to the NH group of the parent compound, or unsubstituted heterocyclylalkyl wherein the alkyl is bonded to the NH group of the parent compound.

29. The compound according to claim 1, wherein R₁ and R₂ are haloalkyl, R₃ is a “trans-cinnamide,” R₄ and R₅ are hydrogen, and Ar is an aryl ring.

30. The compound according to claim 1, wherein R₃ is a “cis-cinnamide” or “trans-cinnamide” and R₁ is not a “cis-cinnamide” or “trans-cinnamide.”

31. The compound according to claim 1, wherein R₃ is a substituent of formula IV and R₁ is not a substituent of formula IV.

32. The compound according to claim 1, wherein R₃ is a cyclopropyl derivative and R₁ is not a cyclopropyl derivative.

33. The compound according to claim 1, wherein R₃ is a substituent of formula VI and R₁ is not a substituent of formula VI.

34. The compound according to claim 1, wherein R₃ is a substituent of formula VII and R₁ is not a substituent of formula VII.

35. The compound according to claim 1, wherein R₁ and R₂ are selected from hydrogen, alkyl, halogen, haloalkyl, and nitro.

36. The compound according to claim 1, wherein R₈ and R₉ are each independently selected from hydrogen, aldehyde, alkanoyl, alkyl, alkylthio, alkenyl, alkynyl, alkoxy, amido, amino, aryl, arylcarbonyl, arylthio, carboxy, cycloalkyl, ester, ether, heterocyclyl, heterocyclylcarbonyl, ketone, nitro, sulfonate, sulfonyl, and thiol, and
when R₁₀ and R₁₁ are not taken together with N to form a heterocyclyl group bonded to at least one substituent, then R₁₀ and R₁₁ are each independently selected from hydrogen, alkyl, alkylthio, alkanoyl, alkenyl, alkynyl, amido, alkoxy, aryl, arylthio, arylcarbonyl, arylalkyl, carboxy, cyano, cycloalkyl, ester, ether, heterocyclyl, heterocyclylcarbonyl, ketone, nitro, and sulfonyl and thiol.

37. The compound according to claim 1, wherein R₁₀ and R₁₁ are each independently selected from alkoxyalkyl, alkoxycarbonylalkyl, alkyl, aryl, carboxyalkyl, cycloalkyl, hydroxyalkyl, heterocyclylalkyl, heterocyclyl, and heterocyclylamino.

38. The compound according to claim 1, wherein R₁₀ and R₁₁ are taken together with N to form a heterocyclyl group bonded to at least one substituent independently selected from alkyl, alkanoyl, alkanoyloxy, alkanoylamino, alkanoyloxyalkyl, alkanoylaminoalkyl, alkoxyalkyl, alkoxycarbonyl, alkoxycarbonylalkyl, amino, alkylsulfonyl, alkylsulfonylaminocarbonyl, arylalkoxycarbonyl, aminoalkyl, aminoalkanoyl, aminocarbonyl, arylsulfonylaminocarbonyl, carboxy, carboxyalkyl, carboxycarbonyl, carboxaldehyde, carboxamido, carboxamidoalkyl, heterocyclyl, heterocyclylalkyl, heterocyclylcarbonyl, heterocyclylalkylaminocarbonyl, hydroxy, hydroxyalkanoyl, hydroxyalkyl, hydroxyalkoxyalkyl, heterocyclylsulfonylaminocarbonyl, and tetrazolyl.

39. The compound according to claim 1, wherein R₁₀ and R₁₁ are taken together with N to form a heterocyclyl group selected from morpholinyl, piperidinyl, piperazinyl, pyridyl, tetrahydropyridyl, and thiomorpholinyl.

40. The compound according to claim 1, wherein the compound exhibits an IC₅₀ of less than or equal to about 1.0 μM as determined by an ICAM-1/LFA-1 biochemical interaction assay.

41-43. (canceled)

44. The compound according to claim 1, wherein the compound exhibits an EC₈₀ of less than or equal to about 3.0 μM as determined by a T cell proliferation assay.

45-46. (canceled)

47. A pharmaceutical composition comprising the compound according to claim 1.

48. (canceled)

49. A method of treating an inflammatory disease or inhibiting inflammation, comprising administering to a subject a pharmaceutical composition comprising the compound of claim 1.

50. A method of treating an immune disease or suppressing an immune response, comprising administering to a subject a pharmaceutical composition comprising the compound of claim 1.

51-52. (canceled)

53. A method of treating a disease associated with an interaction between ICAM-1 and LFA-1, comprising administering to a subject a pharmaceutical composition comprising the compound of claim 1.

54-56. (canceled)

57. A method of treating psoriasis, comprising administering to a subject a pharmaceutical composition comprising the compound of claim 1.

58-61. (canceled)

62. A method for treating a disease or disorder in a mammal, comprising administering to said mammal a therapeutic amount of a compound according to claim 1 or claim 4, wherein the disease or disorder benefits from inhibiting the interaction of LFA-1 with ICAM-1 or ICAM-3, and wherein administering to said mammal inhibits inflammation.

63. A method of inhibiting the interaction of LFA-1 with ICAM-1 or ICAM-3, comprising administering to a mammal an effective amount of a compound according to claim 1 or claim 4, wherein administering to said mammal inhibits inflammation.

64. A method for treating a disease or disorder selected from prophylaxis, reperfusion injury, ischemic-reperfusion injury, pulminary reperfusion injury, stroke, asthma, myocardial infarction, psoriasis, atherosclerosis, atopic dermatitis, hepatitis, adult respiratory distress syndrome, chronic ulceration, lung fibrosis, graft-versus-host disease, chronic obstructive pulmonary disease, Sjögren's syndrome, multiple sclerosis, autoimmune thyroiditis, Graves' disease, glomerulonephritis, systemic lupus erythematosus, diabetes, autoimmune diabetes, primary biliary cirrhosis, autoimmune uveoretinitis, scleroderma, arthritis, Lyme arthritis, fulminant hepatitis, inflammatory liver injury, thyroid diseases, transplant rejection, inflammatory lung injury, radiation pneumonitis, inflammatory bowel diseases, inflammatory glomerular injury, radiation-induced enteritis, peripheral artery occlusion, graft rejection, and cancer, comprising administering to a mammal a therapeutic amount of a compound according to claim 1 or claim 4.
